(19) United States
(12) Patent Application Publication
(10) Pub. No.: US 2022/0304958 A1
(43) Pub. Date: Sep. 29, 2022

(54) SMALL MOLECULE INHIBITORS OF A PROTEIN COMPLEX

(71) Applicants: THE REGENTS OF THE UNIVERSITY OF CALIFORNIA, Oakland, CA (US); THE UNIVERSITY OF NEW MEXICO, Albuquerque, NM (US)

(72) Inventors: Alexandre GINGRAS, La Jolla, CA (US); Mark GINSBERG,, La Jolla, CA (US); Carlo BALLATORE, La Jolla, CA (US); Larry SKLAR, Albuquerque, NM (US); Karol Rogelle Karagdag FRANCISCO, Las Vegas, NV (US)

(21) Appl. No.: 17/611,036

(22) PCT Filed: Jun. 4, 2020
(86) PCT No.: PCT/US2020/036093

§ 371 Date: Nov. 12, 2021

Related U.S. Application Data
(60) Provisional application No. 62/856,849, filed on Jun. 4, 2019.

(51) Int. Cl. A61K 31/166; A61K 31/196; A61K 31/41; A61K 31/4196; A61K 31/4406; A61K 31/245; A61K 31/44; A61K 31/135; A61K 31/423; A61K 31/472; A61K 31/235; A61K 31/05; A61K 31/192; A61K 31/337; A61K 31/085; A61K 31/095; A61K 31/10; A61K 31/055; A61K 31/121; A61K 31/451; A61K 31/136; A61K 31/397; A61K 31/222; A61K 31/137; A61K 31/381; A61K 31/11; A61P 9/14; C07C 251/24; C07D 249/14; C07D 257/06; C07D 213/74; C07C 229/56; C07C 233/81; C07C 251/48; C07D 261/20; C07D 217/16; C07C 215/50; C07C 65/19; C07C 65/105; C07D 305/08; C07C 317/22; C07C 39/38; C07C 49/83; C07D 295/112; C07D 205/08; C07C 69/738; C07C 255/56; C07C 47/575; C07D 333/22

(52) U.S. Cl. CPC A61K 31/166; A61K 31/196; A61K 31/41; A61K 31/4196; A61K 31/4406; A61K 31/245; A61K 31/44; A61K 31/135; A61K 31/423; A61K 31/472; A61K 31/235; A61K 31/05; A61K 31/192; A61K 31/337; A61K 31/085; A61K 31/095; A61K 31/10; A61K 31/055; A61K 31/121; A61K 31/451; A61K 31/136; A61K 31/397; A61K 31/222; A61K 31/137; A61K 31/381; A61K 31/11; A61P 9/14; C07C 251/24; C07D 249/14; C07D 257/06; C07D 213/74; C07C 229/56; C07C 233/81; C07C 251/48; C07D 261/20; C07D 217/16; C07C 215/50; C07C 65/19; C07C 65/105; C07D 305/08; C07C 317/22; C07C 39/38; C07C 49/83; C07D 295/112; C07D 205/08; C07C 69/738; C07C 255/56; C07C 47/575; C07D 333/22

(57) ABSTRACT

Compositions and methods for treating thrombosis, inflammation, and atherosclerosis by administration of a compound that binds to KRIT1 to inhibit binding with HEG1.

### CROSS REFERENCE TO RELATED APPLICATIONS

This application claims the priority benefit of U.S. Provisional Application No. 62/856,849, filed Jun. 4, 2019, which application is incorporated herein by reference.

### TECHNICAL FIELD

The present invention relates to small molecule inhibitors of the HEG1-KRIT1 protein complex.

### BACKGROUND

Endothelial cells (ECs) line the entire circulatory system and EC dysfunction plays a central role in the development of vascular disease states such as atherosclerosis and thrombosis. Heart of Glass (HEG1) is a transmembrane receptor that is required for cardiovascular development in both zebrafish and mammals (1, 7, 8). The cytoplasmic domain (tail) of HEG1 binds directly to KRIT1 (also known as CCM1), the protein product of the KRIT1 gene (3, 8). The interaction recruits KRIT1 to cell-cell junctions thereby anchoring the complex to support heart development in zebrafish (2). Both HEG1 and KRIT1 dampen gene expression levels of transcriptional regulators termed Kruppel-like factors KLF2 and KLF4 (KLF2/4) (14, 47), and therefore play crucial roles in controlling the sensitivity of ECs to hemodynamic forces (15, 48). KLF2/4 are strongly activated within regions of laminar flow and high shear stress (49). In turn, KLF2/4 differentially regulates the expression of factors that confer anti-inflammatory, antithrombotic, and antiproliferative effects in ECs (50). Therefore, inhibiting the HEG1-KRIT1 protein complex increases KLF2/4 expression which has vasoprotective effects useful for the treatment of cardiac disease.

### SUMMARY OF THE INVENTION

The transmembrane protein HEG1 binds directly to and recruits KRIT1 to EC cell junctions to regulate and maintain the organization of junctional molecules, which are critical for vertebrate cardiovascular development (1-4). The crystal structure of the HEG1-KRIT1 protein complex was solved (3, 5) and it was found that the KRIT1 FERM domain binds to the HEG1 cytoplasmic tail C-terminus. This revealed a new mode of FERM domain-membrane protein interaction. The KRIT1 FERM domain consists of three subdomains (F1, F2, and F3) forming a cloverleaf shape in which the F1 and F3 subdomain interface creates a hydrophobic groove that binds the Tyr^{3.380}-Phe^{3.381} of the most C-terminal portion of the HEG1 cytoplasmic tail (2). Moreover, the KRIT1 FERM domain also simultaneously binds Rap1, a small GTPase, on the surface of the F1 and F2 subdomains to stabilize endothelial junctions by forming the HEG1-KRIT1-Rap1 ternary complex (3, 4, 6). This suggests that part of the biological effects of KRIT1, related to endothelial junctional integrity, relies on the KRIT1 FERM domain being recruited to cell-cell junctions to interact with both HEG1 and Rap1.

HEG1 and KRIT1 are also genetically linked in mice (1) and zebrafish during cardiovascular development (1, 7, 8). Krit1^{-/-} mice show gross defects in multiple vascular beds and early embryonic lethality (9). Similarly, Heg1^{-/-} mice result in lethal hemorrhage due to cardiovascular defects (1). Studies in zebrafish embryos show that loss-of-function of krit1 or heg1 leads to vascular dilation and severe heart defects (1, 10, 11). It has been demonstrated that increases in endothelial KLF4 and KLF2 may constitute a major mechanism by which loss of HEG1 or KRIT1 alters cardiovascular development (12-16). Importantly, these changes in KLF4 and KLF2 were associated with the gain of endothelial MAPK/ERK kinase kinase 3 (MEKK3) activity that in turn, upregulates the MEK5-ERK5-MEF2 signaling axis (12-14, 17). Paradoxically, the MEK5-ERK5-MEF2 mechanotransduction module regulates KLF4 and KLF2 expression during laminar blood flow (18, 19) to confer vascular integrity (20) and vasoprotection (21). A study in zebrafish suggested that heg1 and krit1 expression confers cardiovascular development accuracy by fine-tuning endothelial cell response to blood flow (7). These observations suggest that the HEG1-KRIT1 protein complex may be interconnected to mechanosensing proteins (e.g, PECAM1, VE-cadherin, and VEGFR2/3) that respond to flow-induced mechanotransduction (22, 23). Therefore, genetic approaches have contributed enormously to the understanding of the fundamental molecular and cellular processes regulated by endothelial HEG1 and KRIT1 proteins. Before this invention, it remained to be clarified whether the effect of inhibition of the endothelial HEG1-KRIT1 interaction leads to similar outcomes such as loss of HEG1 or KRIT1.

In this invention a high-throughput screen followed by structure-function based optimization of a new class of inhibitors of the HEG1-KRIT1 interaction was performed to uncover acute changes in signaling pathways downstream of the HEG1-KRIT1 protein complex. It was discovered that HKi2 is a bona fide inhibitor by competing orthosterically with HEG1 for binding to the KRIT1 FERM domain. The crystal structure of HKi2 bound to KRIT1 FERM reveals that it occupies the same binding pocket on KRIT1 as the HEG1 cytoplasmic tail. In human endothelial cells (EC), acute inhibition of the HEG1-KRIT1 interaction by HKi2 triggers PI3K/Akt signaling. HKi2-treated cells also increase KLF4 and KLF2 mRNA within 4 hours, whereas a structurally-similar inactive compound failed to do so. In zebrafish, HKi2 induces expression of klf2a in arterial and venous endothelium. Furthermore, genome-wide RNA transcriptome analysis of HKi2-treated ECs under static conditions reveals that, in addition to elevating KLF4 and KLF2 expression, inhibition of the HEG1-KRIT1 interaction mimics many of the transcriptional effects of laminar blood flow. Thus, this invention demonstrates that acute inhibition of the HEG1-KRIT1 interaction activates PI3K/Akt activity and elevates KLF4 and KLF2 gene expression. Thus, HKi2 provides a new pharmacologic tool to study acute inhibition of the HEG1-KRIT1 protein complex and may provide insights to dissect the relationship of the HEG1-KRIT1 complex to mechanosensing proteins that respond to flow-induced mechanotransduction.

Moreover, because vasoprotection can be achieved by pharmacological disruption of the HEG1-KRIT1 complex in the endothelium, via the elevation of KLF4/2, the methods and compositions disclosed in this invention can be used in the treatment of inflammatory diseases, thrombosis, or atherosclerosis.

The disclosure provides a method of inhibiting thrombosis or inflammation in a subject comprising administering to a subject in need an effective amount of a compound that binds to KRIT1 FERM domain to inhibit binding with HEG1. In embodiments, the invention provides a method of inducing expression of KLF2/4 comprising administering to a subject in need an effective amount of a compound that binds to KRIT1 FERM domain to inhibit binding with HEG1.

In embodiments, the invention provides a compound or Formula (A) or Formula (B), a salt thereof, or a salt hydrate thereof;

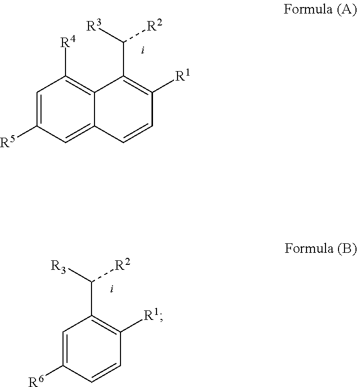

wherein R¹ is selected from the group consisting of hydroxyl and hydrogen;

wherein R² is selected from the group consisting of oxygen and nitrogen, wherein the nitrogen is substituted with (a) R^{a} or (b) R^{a} and R^{b}, wherein i is (i) a single bond, a double bond, or a triple bond when R² is nitrogen, or (ii) a double bond when R² is oxygen;

wherein R³ is selected from the group consisting of hydrogen and a C₁-C₂₀ hydrocarbyl;

wherein R⁴ is selected from the group consisting of hydrogen, hydroxyl, nitrogen, and oxygen, wherein the oxygen is substituted with R^{c}, and the nitrogen is substituted with (i) R^{d} or (ii) R^{d} and R^{e};

wherein R⁵ is selected from the group consisting of (i) hydrogen, (ii) hydroxyl, (iii) a C₁-C₂₀ hydrocarbyl, (iv) a halogen, (v) nitrogen, and (vi) oxygen, wherein the oxygen is substituted with R^{f}, and the nitrogen is substituted with (a) R^{g} or (b) R^{g} and R^{h}; and

wherein R⁶ is selected from the group consisting of hydrogen and a C₁-C₂₀ hydrocarbyl;

wherein R^{c} and R^{f} are independently selected from a C₁-C₂₀ hydrocarbyl, and

wherein R^{a}, R^{b}, R^{d}, R^{e}, R^{g} and R^{h} are independently selected from hydrogen and a C₁-C₂₀ hydrocarbyl.

In embodiments, R^{c} and R^{f} are independently selected from a C₁-C₁₀ hydrocarbyl, and R^{a}, R^{b}, R^{d}, R^{e}, R^{g} and R^{h} are independently selected from hydrogen and a C₁-C₁₀ hydrocarbyl.

In embodiments, R^{c} and R^{f} are independently selected from a C₁-C₆ hydrocarbyl, and R^{a}, R^{b}, R^{d}, R^{e}, R^{g} and R^{h} are independently selected from hydrogen and a C₁-C₆ hydrocarbyl

In embodiments, R¹ is hydroxyl, and R⁴ and R⁵ are hydrogen. In embodiments, R² is nitrogen, R³ is hydrogen, and i is a double bond. In embodiments, R^{a} is further selected from the group consisting of o-benzoic acid, m-benzoic acid, p-benzoic acid, and 5-(1H-tetrazole).

In embodiments, R² is oxygen, R³ is hydrogen, and i is a double bond. In embodiments, R¹ is hydroxyl, and R⁴ and R⁵ are hydrogen.

In embodiments, R¹, R⁴, and R⁵ are hydrogen. In embodiments, R² is oxygen, R³ is hydrogen, and i is a double bond.

In embodiments, R¹ is hydroxyl, R² is oxygen. R³ is hydrogen, and i is a double bond. In embodiments, R⁶ is a phenyl.

In embodiments, R² is oxygen, R³ is hydrogen, and i is a double bond. In embodiments, R⁵ is hydroxyl. In embodiments, R⁵ is a methyl. In embodiments, R⁵ is oxygen. In embodiments, R^{f} is a methyl. In embodiments. R⁵ is an acetyl. In embodiments, R⁵ is chloro. In embodiments, R⁵ is a piperidinyl. In embodiments, R⁵ is nitrogen, R^{g} is a methyl, and R^{h} is a methyl. In embodiments, R⁵ is an azetidinyl. In embodiments, R⁵ is a propen-1-yl, an ethyl, an ethenyl, or an ethynyl. In embodiments, the ethenyl is substituted with an ethyl ester. In embodiments, the ethyl is substituted with a hydroxyl. In embodiments, R⁴ is hydroxyl, and R⁵ is hydrogen.

In some embodiments, the compound is a compound of Formula (A), wherein R¹, R³, R⁴, and R⁵ are hydrogen, R² is oxygen, i is a double bond, and the compound is 1-naphthaldehyde:

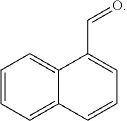 [SMILES: O=Cc1cccc2ccccc12]

In some embodiments, the compound is a compound of Formula (A), a salt thereof, or a salt hydrate thereof, wherein R¹ is hydroxyl, and the compound has a structure according to Formula (A1):

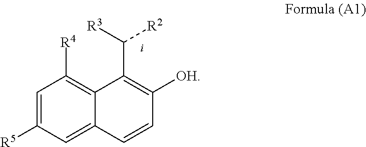

In some embodiments, the compound is a compound of Formula (A1), wherein R⁴ and R⁵ are hydrogen, and the compound has a structure according to Formula (A2):

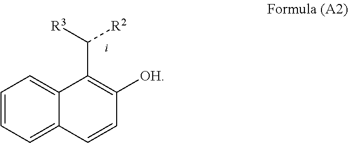

In some embodiments, the compound is a compound of Formula (A2), wherein R² is oxygen, R³ is hydrogen, i is a double bond, and the compound is 2-hydroxy-1-naphthaldehyde, which has the following structure:

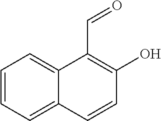 [SMILES: O=Cc1c(O)ccc2ccccc12]

In some embodiments, the compound is a compound of Formula (A2), wherein R² is nitrogen, R³ is hydrogen, i is a double bond, and the compound has a structure according to Formula (A³):

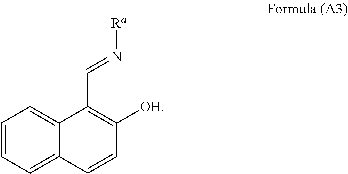

In some embodiments, the compound is a compound of Formula (A3), wherein R^{a} is a C₁-C₆ hydrocarbyl. In some embodiments, R^{a} is a benzoic acid, and the compound has a structure according to Formula (A4):

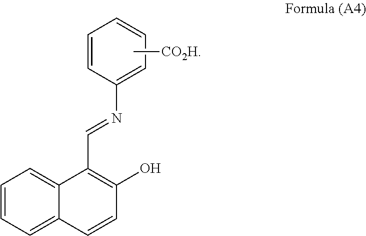

In some embodiments, the compound is a compound of Formula (A4), wherein the benzoic acid substituent is an o-benzoic acid substituent, a m-benzoic acid substituent, or a p-benzoic acid substituent, and the compound, respectively, is (E)-2-(((2-hydroxynaphthalen-1-yl)methylene)amino)benzoic acid, (E)-3-(((2-hydroxynaphthalen-1-yl)methylene)amino)benzoic acid, or (E)-4-(((2-hydroxynaphthalen-1-yl)methylene)amino)benzoic acid.

In some embodiments, the compound is a compound of Formula (A3), wherein R^{a} is a H-tetrazolyl, and the compound is (E)-1-(((1H-tetrazol-5-yl)imino)methyl)naphthalen-2-ol:

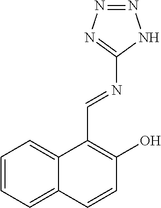 [SMILES: Oc1ccc2ccccc2c1/C=N/c1nnn[nH]1]

In some embodiments, the compound is a compound of Formula (A1), wherein R² is oxygen, R³ is hydrogen, i is a double bond, R⁴ is hydrogen, and the compound has a structure according to Formula (A5):

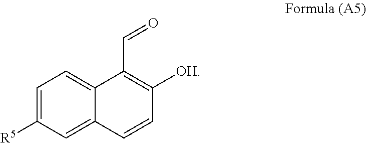

In some embodiments, the compound is a compound of Formula (A5), wherein R⁵ a C₁-C₁₀ hydrocarbyl, or a C₁-C₆ hydrocarbyl. In some embodiments, the compound is a compound of Formula (A5), wherein R¹ is selected from the substituents provided at the following Table 1, which result in the corresponding compounds.

TABLE 1

| R⁵ | Structure/Name |
|---|---|
| Hydroxyl | 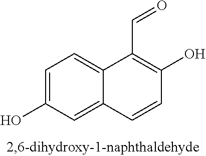 [SMILES: O=Cc1c(O)ccc2cc(O)ccc12] |
| Methyl (an unsubstituted C₁ hydrocarbyl) | 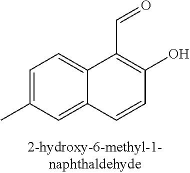 |
| Oxygen, wherein R^{d} is methyl (an unsubstituted C₁ hyrdrocarbyl) | 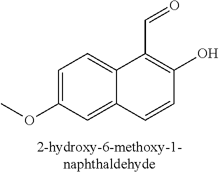 |
| Acetyl (a C₂ hydrocarbyl substituted with an oxygen atom to form an oxo group) | 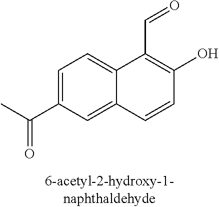 [SMILES: CC(=O)c1ccc2c(C=O)c(O)ccc2c1] |
| Chloro | 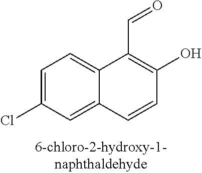 [SMILES: O=Cc1c(O)ccc2cc(Cl)ccc12] |
| Piperidin-1-yl (a C₅ hydrocarbyl heterocycle including one nitrogen heteroatom) | 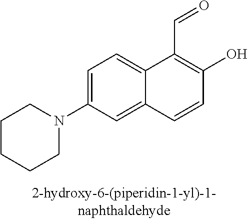 [SMILES: O=Cc1c(O)ccc2cc(N3CCCCC3)ccc12] |
| Nitrogen, wherein R^{f} and R^{g} are methyl. | 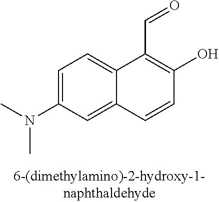 [SMILES: CN(C)c1ccc2c(C=O)c(O)ccc2c1] |
| 2-oxoazetidin-1-yl (a substituted C₃ hydrocarbyl heterocycle including one nitrogen heteroatom) | 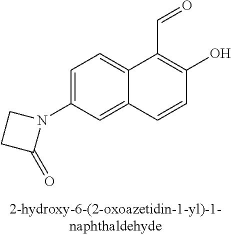 [SMILES: O=Cc1c(O)ccc2cc(N3CCC3=O)ccc12] |
| Allyl (propen-1-yl) | 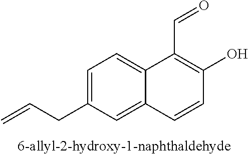 [SMILES: C=CCc1ccc2c(C=O)c(O)ccc2c1] |
| Ethyl (an unsubstituted C₂ hydrocarbyl) | 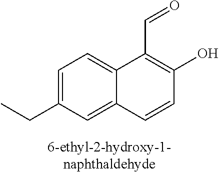 |
| Ethyne (an unsubstituted C₂ hydrocarbyl) | 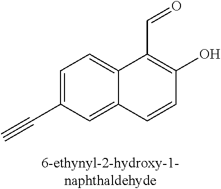 |
| Butyl (an unsubstituted linear C₄ hydrocarbyl) | 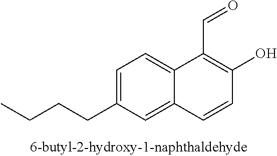 [SMILES: CCCCc1ccc2c(C=O)c(O)ccc2c1] |
| Ethyl acetate- substituted ethenyl | 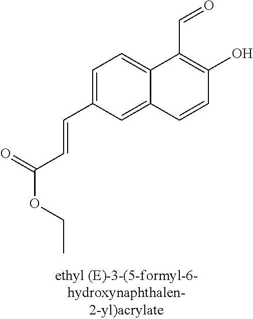 [SMILES: CCOC(=O)/C=C/c1ccc2c(C=O)c(O)ccc2c1] |
| Hydroxy-substituted ethyl | 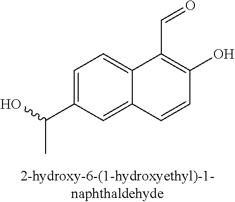 [SMILES: CC(O)c1ccc2c(C=O)c(O)ccc2c1] |

In some embodiments, the compound is a compound of Formula (A1), wherein R² is oxygen, R³ is hydrogen, i is a double bond, R⁵ is hydrogen, and the compound has a structure according to Formula (A6):

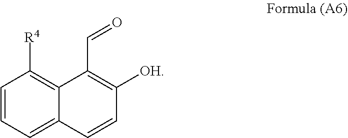

In some embodiments, the compound is a compound of Formula (A6), wherein R⁴ is oxygen, R^{c} is methyl, and the compound is 2-hydroxy-8-methoxy-1-naphthaldehyde:

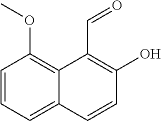

In some embodiments, the compound is a compound of Formula (A6), wherein R⁴ is hydroxyl, and the compound is 2,8-dihydroxy-1-naphthaldehyde:

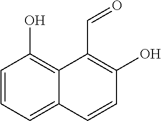 [SMILES: O=Cc1c(O)ccc2cccc(O)c12]

In some embodiments, the compound is a compound of Formula (B), wherein R¹ is hydroxyl, R² is oxygen, R³ is hydrogen, R¹ is a phenyl, i is a double bond, and the compound is 4-hydroxy-[1,1′-biphenyl]-3-carbaldehyde:

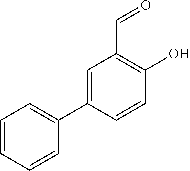 [SMILES: O=Cc1cc(-c2ccccc2)ccc1O]

In embodiments, this invention discloses a method of treating a disease in a subject by reducing thrombosis, atherosclerosis, or inflammation comprising administering to a subject in need an effective amount of a Siritol compound or salt thereof that binds to KRIT1 FERM domain to inhibit binding with HEG1.

In embodiments, the disease is rheumatoid arthritis, gout, spondyloarthritis, vasculitis, adult respiratory distress syndrome, post-perfusion injury, glomerulonephritis, cytokine storm, myocardial infarction, stroke, deep vein thrombosis, pulmonary embolus, thrombotic thrombocytopenic purpura, COVID-19, coronary artery disease, carotid atherosclerosis, cerebrovascular disease, vascular dementia, or aortic aneurysm.

In embodiments, the compound is a compound or Formula (A) or Formula (B), a salt thereof, or a salt hydrate thereof.

In embodiments, the compound is selected from the group consisting of HKi1, HKi2, HKi5, BL-0549, BL-0558, BL-0552, BL-0628, BL-0661, BL-0666, BL-0670, BL-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

In embodiments, the Sirtinol derivative comprises an aldehyde moiety.

In embodiments, the compound upregulates endothelial nitric oxide synthase, thrombomodulin 1, vascular endothelial growth factor A, Thrombospondin 1, Monocyte chemoattractant protein, or C-X-C chemokine receptor type 4. In embodiments, the compound upregulates PI3K/Akt signaling.

In embodiments, the compound occupies a HEG1 binding pocket of KRIT1. In embodiments, the administering induces expression of KLF2 or KLF4.

In embodiments, this invention discloses a method of improving laminar blood-flow in a subject comprising administering to a subject in need an effective amount of a Sirtinol compound or salt thereof that binds to KRIT1 FERM domain to inhibit binding with HEG1. In embodiments, the compound is selected from the group consisting of HKi1, HKi2, HKi5, BL-0549, BL-0558, BL-0552, BL-0628, BL-0661. BL-0666, BL-0670, BL-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

In embodiments, this invention discloses a compound or Formula (A) or Formula (B), a salt thereof, or a salt hydrate thereof. In embodiments, this invention discloses a pharmaceutical composition comprising a treatment effective amount of a compound chosen from the group consisting of Formula (A) or Formula (B), a salt thereof, or a salt hydrate thereof. In embodiments, the compound is chosen from the group consisting of HKi3, BL-0549, BL-0558, BL-0552, BL-0628, BL-0661, BL-0666, BL-0670, BI-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

In embodiments, the composition is used to reduce thrombosis, atherosclerosis, or inflammation in a subject in need. In embodiments, the HEG1-KRIT1 protein complex is inhibited.

### BRIEF DESCRIPTION OF THE DRAWINGS

FIGS. 1A-1F show a flow cytometry assay for the HEG1-KRIT1 FERM domain interaction.

FIGS. 2A-2B show that HKi1 is an inhibitor of the HEG1-KRIT1 interaction.

FIGS. 3A-3F show structure guided HEG1-KRIT1 interaction inhibitors.

FIG. 4 shows that aldehyde in position C1 and hydroxyl group in position C2 are important for HKi2 activity.

FIGS. 5A-5D show KRIT1 lysine residues are important for HKi2 activity and HKi2 does not block PARD3 binding to HEG1.

FIGS. 6A-6F show that HKi2 treatment activated PI3K/Akt signaling and leads to KLF2 and KLF4 upregulation in endothelial cells.

FIGS. 7A-7C show that HKi2 treatment leads to KLF4 and KLF2 upregulation, and their important transcriptional targets.

FIGS. 8A-8B show HKi2 induces expression of klf2a in arterial and venous endothelium in zebrafish.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises.” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B. and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients. e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a“pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

The phrase “C₁-C₂₀ hydrocarbyl,” “C₁-C₁₀ hydrocarbyl,” “C₁-C₆ hydrocarbyl,” or the like, as used herein, generally refer to an aliphatic group, an aromatic or aryl group, a cyclic group, a heterocyclic group, or any combination thereof, including any substituted derivative thereof, such any halo-, alkoxy-ester-substituted, or amide-substituted derivative thereof, having 1 to 30 carbon atoms, 1 to 20 carbon atoms, or 1 to 5 carbon atoms, or the like. Also included in the definition of “C₁-C₂₀ hydrocarbyl,” “C₁-C₁₀ hydrocarbyl,” “C₁-C₆ hydrocarbyl,” or the like, are any unsubstituted, branched, or linear analogs thereof. The “C₁-C₂₀ hydrocarbyl,” “C₁-C₁₀ hydrocarbyl,” “C₁-C₆ hydrocarbyl,” or the like, may be substituted, as described below, with one or more functional moieties, which include a halide, an ether, a ketone, an ester, an amide, a nitrile, a heterocycle comprising at least one N-, O-, or S-heteroatom, an aldehyde, a thioether, an imine, a sulfone, a carbonate, a urethane, a urea, or an imide. The “C₁-C₂₀ hydrocarbyl,” “C₁-C₁₀ hydrocarbyl,” “C₁-C₆ hydrocarbyl,” or the like, also may include one or more silicon atoms.

Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof, in each instance having from 1 to about 20 carbon atoms, 1 to 10 carbon atoms, 1 to 6 carbon atoms, or the like. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., l-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Examples of aryl or aromatic moieties include, but are not limited to, anthracenyl, azulenyl, biphenyl, fluorenyl, indan, indenyl, naphthyl, phenanthrenyl, phenyl, 1,2,3,4-tetrahydro-naphthalene, and the like, including substituted derivatives thereof, in each instance having from 3 to 30 carbons. Substituted derivatives of aromatic compounds include, but are not limited to, tolyl, xylyl, mesityl, and the like, including any heteroatom substituted derivative thereof. Examples of cyclic groups, in each instance, include, but are not limited to, cycloparaffins, cycloolefins, cycloacetylenes, arenes such as phenyl, bicyclic groups and the like, including substituted derivatives thereof, in each instance having from 3 to about 20 carbon atoms. Thus heteroatom-substituted cyclic groups such as furanyl are also included herein.

In each instance, the heterocyclic compound or heterocycle includes at least one N-, O-, or S-heteroatom, and may be selected from the group consisting of oxetanyl, azetidinyl, thietanyl, thiophenyl, morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide, and homothiomorpholinyl S-oxide, pyridinyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, or benzothiopyranyl S,S-dioxide.

Unless otherwise indicated, the term “substituted,” when used to describe a chemical structure or moiety, refers to a derivative of that structure or moiety wherein one or more of its hydrogen atoms is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), primary amine, secondary amine, tertiary amine (such as alkylamino, arylamino, arylalkylamino, a nitrogen atom of a nitrile, etc.), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxygen (e.g., an oxygen atom of an oxo group, the oxo group being formed by the oxygen atom substituent and the carbon atom substituted with the oxygen atom), phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

### EXAMPLES

The Kruppel-like Factors 2 and 4 (KLF2/4) are transcription factors and master regulators of endothelial cells (ECs) phenotype and homeostasis. KLF2/4 are important blood-flow-responsive genes within ECs that differentially regulate the expression of factors that confer anti-inflammatory, antithrombotic, and antiproliferative effects in ECs. This invention demonstrates that genetic inactivation of endothelial KRIT1 (Krev interaction trapped protein 1) or HEG1 (Heart of glass) leads to upregulation of KLF2/4 expression levels. This invention also discloses that vasoprotective proteins, such as endothelial nitric oxide synthase (eNOS) and thrombomodulin (TM), are upregulated by the increase of KLF2/4 as a result of loss of endothelial KRIT1.

A high-throughput screening assay was developed to identify inhibitors of the HEG1-KRIT1 interaction and identified sirtinol (HKi1) as a promising hit inhibitor. The crystal structure of sirtinol bound to the KRIT1 FERM domain confirmed the primary screening results and ultimately led to the identification of a fragment-like inhibitor (HKi2), which occupies the HEG1 pocket producing comparable activity. These findings suggest that these inhibitors block the interaction by competing with the HEG1 for binding to KRIT1 FERM domain. Moreover, these results demonstrate that HKi2 upregulates KLF2/4 gene expression in two types of human ECs. These results reveal an unexpected role of inhibiting the HEG1-KRIT1 interaction and provide a proof-of-concept that pharmacological manipulation of this complex offers new opportunities to induce expression of KLF214 as vasoprotective factors.

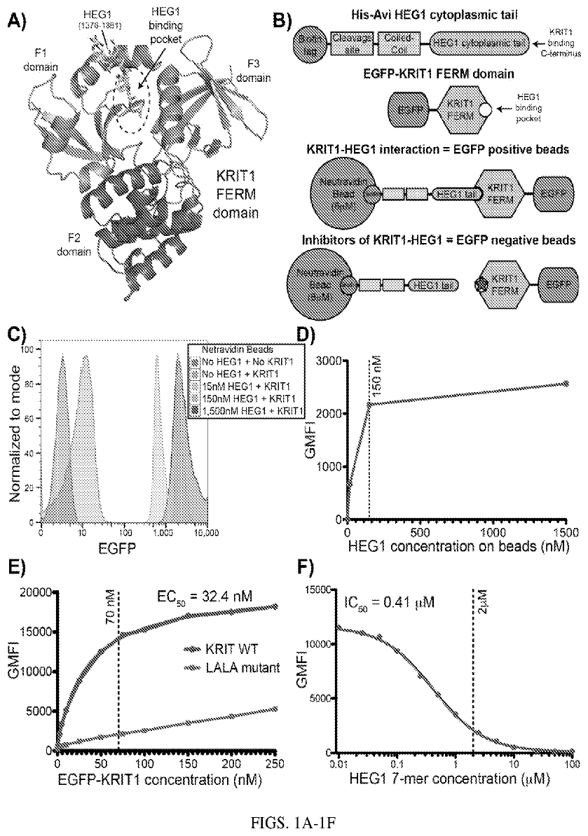

High-throughput screening identifies inhibitors of HEG1-KRIT1 protein interaction. The crystal structure of the KRIT1 FERM domain bound to the C-terminal region of the HEG1 cytoplasmic tail (FIG. 1A) (2) was previously solved. Because the HEG1 binding pocket on the KRIT1 FERM domain is both discrete and unique, it was hypothesized that specific inhibitors of the HEG1-KRIT1 protein complex could be identified. Therefore, a high-throughput flow cytometry-screening assay was developed to screen for compounds that block the HEG1-KRIT1 protein interaction. It was previously shown that the HEG1 cytoplasmic tail can be used as an affinity matrix for KRIT1 binding (2) and this matrix was used to identify important interactors for HEG1 function such as Rasip1 (26).

Using a similar approach, the biotinylated HEG1 cytoplasmic tail (a.a. 1274-1381) peptide was coupled to 6-micron SPHERO Neutravidin coated particles (FIG. 1B). Varying amounts of biotinylated HEG1 peptide was added to the beads (FIG. 1C) and addition of purified recombinant GFP-KRIT1 FERM domain to the HEG1 matrix beads, without washes, leads to a dose dependent GFP intensity increase by flow cytometry (FIG. 1D). Importantly, many beads formed doublets at a 1,500 nM HEG1 concentration in the light scattering affecting the GFP signal (FIG. 1C). Therefore, a concentration of 150 nM biotinylated HEG1 was used for the assay, which gives the best signal without aggregation of the beads. Secondly, addition of increasing amounts of purified recombinant GFP-KRIT1 FERM domain to the HEG1 matrix beads, without washes, lead to a dose dependent GFP intensity increase by flow cytometry with EC50=6.7 nM (FIG. 1E), showing that GFP-KRIT1 binds the HEG1 tail on the beads. Importantly, a KRIT1 (L717,721A) mutant with a >100-fold reduction in HEG1 affinity (4), showed almost no binding at concentration below 50 nM (FIG. 1E), validating this approach and showing specific binding. Therefore, a concentration of 70 nM for the was used assay.

Previous data using Isothermal Titration Calorimetry (ITC) showed a KD=1.2 μM for the KRIT1 FERM domain binding to a HEG1 peptides in solution (4). Using the HEG1 matrix beads an EC50=6.7 nM (FIG. 1E) was observed. The measured apparent off-rate is slower than the actual off-rate, because following dissociation the GFP-KRIT1 can bind to an unoccupied HEG1 tail before diffusing out of the matrix. Importantly, incubation of the GFP-KRIT1 FERM domain with a non-biotinylated HEG1 C-terminus 7-mer peptide block the interaction in a dose dependent manner with IC50=410 nM (FIG. 1F). Again, it was observed that the concentration of peptide in solution to block the interaction is relatively higher than expected due to the slower off-rate explained by the nature of the HEG1 matrix. These results show a reliable and quantitative assay to study the HEG1-KRIT1 protein interaction by flow cytometry.

Specifically. FIGS. 1A-IF show a flow cytometry assay for the HEG1-KRIT1 FERM domain interaction. FIG. 1A is a ribbon diagram of KRIT1 FERM domain in complex with the HEG1 cytoplasmic tail (PDB ID: 3u7d). The KRIT1 FERM domain consists of three subdomains: F1, F2, and F3. The feature of the F1 domain that is not present in other FERM domain is shown in grayscale and that region is an important part of the HEG1 binding pocket. FIG. 1B is a schematic representation of the HEG1 cytoplasmic tail (a.a. 1274-1381) peptide coupled to Neutravidin beads and the EGFP-KRIT1 FERM domain. Binding of EGFP-KRIT1 FERM domain to the HEG1 matrix beads can be detected by flow cytometry. Small molecule inhibitors HKi preventing the interaction of EGFP-KRIT1 FERM domain with the HEG1 matrix beads reduce the EGFP fluorescence signal. FIG. 1C is a flow cytometry profile of SPHERO Neutravidin Polystyrene Particles coated with increasing amount of biotinylated HEG1 peptide and 150 nM EGFP-KRIT1 FERM domain. Many beads doublets in the light scatter signal at 1,500 nM concentration of HEG1 peptide. FIG. 1D is a titration curve for the interaction of EGFP-KRIT1 FERM domain with increasing amounts of HEG1 on the beads as shown in FIG. 1C. The 150 nM HEG1 peptide concentration was used for future experiments. FIG. 1E is a titration curve for the interaction of 150 nM HEG1 on the beads with increasing amounts of EGFP-KRIT1 FERM domain (0-250 nM) wild-type and KRIT1 (L717,721A) mutant. The 70 nM EGFP-KRIT1 concentration was used for future experiments. FIG. 1G is a competition binding curve of 70 nM EGFP-KRIT1 FERM domain binding to 150 nM HEG1 on the beads with increasing amounts on non-biotinylated HEG1 7-mer peptide. The 2 μM HEG1 7-mer concentration was used for future experiments.

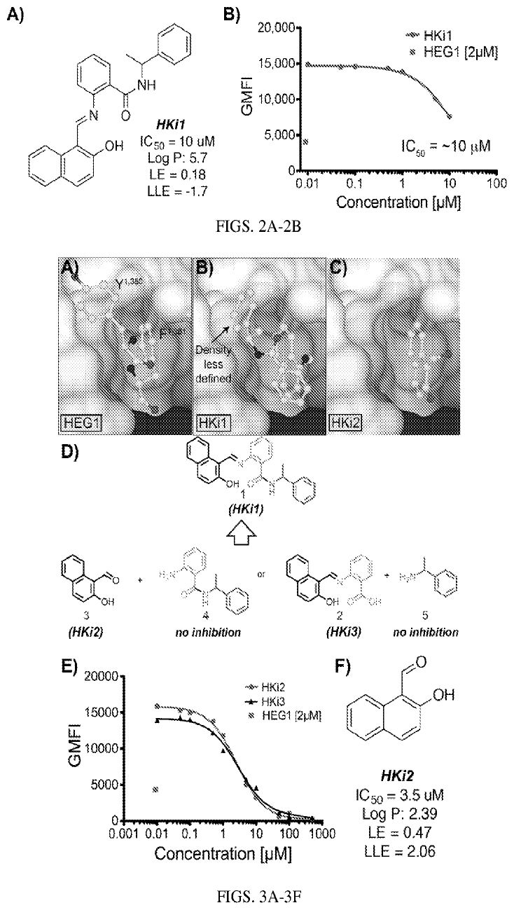

High-throughput screening identifies inhibitors of HEG1-KRIT1 protein interaction. Since the flow cytometry assay to study the HEG1-KRIT1 interaction is simple, requires no washes, and can be inhibited using a HEG1 peptide, the assay was scaled down for high throughput in 384-well plate format. The assay required only 10 μl of sample per well in nanomolar concentrations with a count of 1,000 beads per microliter. A pilot screen was performed using an automated sample loader attached to a flow cytometer and analyzed 2 μl of sample per well (2,000 beads). By alternating beads with GFP-KRIT1 in the absence or presence of 2 μM HEG1 7-mer blocking peptide (Sup. FIG. 1A) a Z′ of 0.528 was measured, classifying the assay as excellent (28). Out of 6,026 compounds screened HEG1-KRIT1 inhibitor 1 (HKi1), also known as Sirtinol was identified (FIG. 2A). Hki1 was originally identified as an inhibitor of sirtuin NAD⁺-dependent deacetylases (1416), and had promising pharmacological properties with an IC₅₀ value of ˜10 μM (FIGS. 2A-2B). However, consistent with a high log P value of 5.7 (FIG. 2A), HKi1 had limited aqueous solubility at 50 μM concentrations or higher in the buffer conditions. As a result, saturating conditions in the assay could not be achieved (FIG. 2B).

Specifically, FIGS. 2A-2B show that HKi1 is an inhibitor of the HEG1-KRIT1 interaction. FIG. 2A shows the chemical structure of HKi1. LE=(1.37/HA)×pIC₅₀ where HA is the number of non H atoms present in the ligand; LLE=pIC₅₀−Log P. FIG. 2B shows the competition binding curve of 70 nM EGFP-KRIT1 FERM domain binding to 150 nM HEG1 on the beads with increasing amounts of HKi1. HKi1 had poor solubility in buffer and concentrations >30 μM could not be reached.

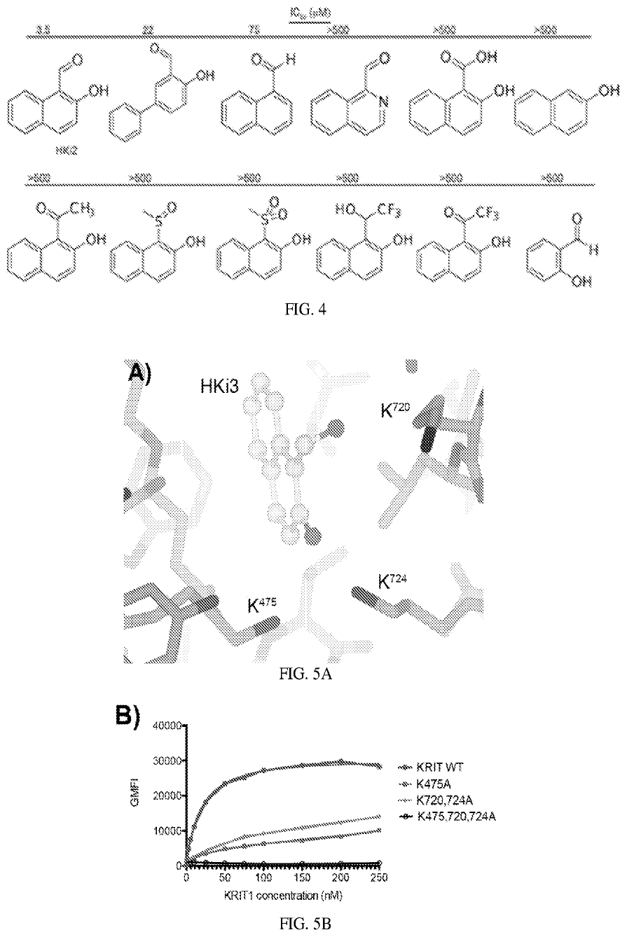

Crystal structure of KRIT1 FERM domain in complex with HKi1. The crystal structure of the KRIT1 FERM domain bound to a HEG1 peptide was previously determined (2) (FIGS. 1A and 3A), the KRIT1 FERM domain was then crystallized in the presence of HKi1 and the structure of the complex was solved to 1.75 Å resolution. The structure confirmed that this compound occupies the same pocket as the HEG1 (FIG. 3B), supporting that HKi1 blocks the interaction by competing orthosterically with the HEG1 for binding to KRIT1 FERM domain. HKi1 is mostly hydrophobic (log P=5.7), as the HEG1 C-terminal Tyr-Phe residues, and sits in the hydrophobic pocket formed at the interface of the F1 and F3 subdomains of the KRIT1 FERM domain. Interestingly, good electron density was observed for approximately half of the molecule, and less well-defined electron density was observed for the other half of the molecule (FIG. 3B), suggesting that modifications to HKi1 could improve binding properties.

HKi2, an HKi1 fragment, blocks HEG1-KRIT1 protein interaction. In addition to the relatively high lipophilicity and low aqueous solubility, HKi1 is also characterized by suboptimal values in efficiency metrics, such as the ligand efficiency (LE) and the lipophilic ligand efficiency (LLE). These parameters are commonly used in drug discovery to facilitate the selection and optimization of fragments, hits and leads (34, 35). Interestingly, analysis of the complex structure (FIG. 3B) suggested that while the naphthalene moiety of HKi1 may play an important role in determining the compound's binding and inhibitory activity, other fragments (e.g., the benzylamine) may not be as intimately involved in the binding to KRIT1. This observation led to the deconstruction HKi1 into its constituent fragments (FIG. 3D) and the investigation of the ability of these fragments to inhibit the HEG1-KRIT1 in vitro.

These studies confirmed that sub-structures containing the substituted naphthalene fragment, such as HKi2 and HKi3, produced inhibition of the HEG1-KRIT1 interaction with IC50 values of 3.5 μM that are closely comparable to the IC50 value of the parent compound. HKi1 (FIG. 3E). Interestingly, when the KRIT1 FERM domain in complex was crystallized with HKi2 (FIG. 3C), it was observed that the naphthalene fragment retained the same binding mode within the HEG1 binding pocket on KRIT1 (FIG. 3A) as noted in the HKi1 complex (FIG. 3B). Given the relatively small size and reduced lipophilicity of HKi2 (FIG. 3F), the LE, as well as the LLE, are considerably improved, suggesting that HKi2 is a promising starting point for further optimization.

Specifically, FIGS. 3A-3F show structure guided HEG1-KRIT1 interaction inhibitors. FIG. 3A is a surface charge representation of KRIT1 FERM domain in complex with the HEG1 cytoplasmic tail (PDB ID: 3u7d). The HEG1 peptide is shown with the C-terminal Tyr-Phe sitting in the binding pocket. FIG. 3B is the crystal structure of the KRIT1 FERM domain in complex with HKi1. The small naphthalene is sitting in the same pocket as the Phe of HEG1 and the electron density for the benzylamine moiety is less defined. FIG. 3C is the crystal structure of the KRIT1 FERM domain in complex with HKi2. The small naphthalene is sitting in the same pocket as the Phe of HEG1. FIG. 3D shows the chemical structure of HKi1 constituents. FIG. 3E shows the competition binding curve of 70 nM EGFP-KRIT1 FERM domain binding to 150 nM HEG1 on the beads with increasing amounts on HKi2 and HKi3. FIG. 3F show the chemical structure of HKi2. LE and LLE are described in FIG. 2A. The solubility of HKi2 in aqueous solution is largely improved.

Evaluation of structure activity relationship. To investigate the structure activity relationship (SAR) of HKi2, a focused set of analogues were either purchased or synthesized (See, FIG. 4 and Table 2) and then tested in the in vin assay. Compounds lacking the aldehyde moiety had no inhibitory activity detected by flow cytometry-screening assay (i.e., IC₅₀ of >500 μM), suggesting that the aldehyde plays a critical role.

Specifically, FIG. 4 shows that the aldehyde in position C1 and hydroxyl group in position C2 are important for HKi2 activity. The IC₅₀ was measured using flow cytometry-screening assay. N.I.=no inhibition detected up to 500 μM concentration thus IC₅₀>500 μM.

In addition, removal of the hydroxyl group (1-naphthaldehyde) resulted in weak inhibition with an IC₅₀ of 75 μM, while no inhibition was observed for compound BL-0607 suggesting that the hydroxyl group at C2 is also preferred for inhibition activity. This observation is consistent with the presence of a hydrogen bond between the hydroxyl moiety of HKi2 and the side chain of Lys⁷²⁴ that was observed in the crystal structure (FIG. 5A). Finally, although opening of the fused bicyclic naphthalene ring of HKi2 to the corresponding non-fused phenylbenzene system BL-0628, resulted in retention of moderate inhibition activity, with an IC₅₀ of 22 μM, interestingly, salicylic aldehyde (Salicylaldehyde) did not exhibit detectable activity in the assay suggesting that extended bi-cyclic aromatic systems may be ultimately preferred for inhibition of the HEG1-KRIT1 interaction. Thus, these results indicate that although the reactive carbonyl group in C1 is clearly required for inhibition of the HEG1-KRIT1 interaction, other features, such as the hydroxyl group in position C2 and a relatively large aromatic scaffold also play an important role.

Lysine residues in KRIT1 are important for inhibition. The crystal structure shows that the HEG1-binding pocket of KRIT1 contains three lysines residues (Lys⁴⁷⁵, Lys⁷²⁴, and Lys⁷²⁰) in the vicinity of the hydroxy-aldehyde of HKi2 (FIG. 5A). Although it is conceivable that the aldehyde moiety of HKi2 may engage in covalent reversible binding with one of these residues leading to the relatively potent inhibition of the HEG1-KRIT1 interaction, the electron density for the three lysines side-chains is poorly resolved, so direct evidence of covalent modification of these amino acid residues has not been obtained. Nonetheless, mutation of any of the three KRIT1 lysines residues reduced the KRIT1 binding considerably to HEG1 (FIG. 5B), suggesting that these residues are important for the protein-protein interaction. This suggests that the inhibition produced by hydroxy naphthaldehyde compounds, such as HKi2, may be mediated by the interactions that these compounds establish with the lysines residues of KRIT1.

Specially, FIGS. 5A-5D show that KRIT1 lysine residues are important for HKi2 activity and HKi2 does not block PARD3 binding to HEG1. FIG. 5A shows KRIT1 bound to HKi2 highlighting the position of three lysines residues near the HKi2 aldehyde. FIG. 5B shows that all tested EGFP-KRIT1 FERM domain mutants tested had reduced HEG1 binding. FIG. 5C shows HUVEC lysates were incubated with either HEG1 WT or HEG1 ΔYF matrix and Western blotted for PARD3. The mixture contained either DMSO, HKi2 or the inactive compound. The binding to HEG1 ΔYF is largely reduced in comparison to HEG1 WT, but neither HKi2 nor an inactive compound, 2-hydroxy-1-naphthoic acid, affected the binding. FIG. 5D shows relative PARD3 binding from three independent experiments. Mean with SD are shown. ANOVA with a Tukey post hoc test: *, P<0.05.

HKi2 blocks the HEG1-KRIT1, but not the HEG1-PARD3 interaction. To further test the specificity of this invention's compound to block the HEG1-KRIT1 interaction, but not other proteins, a previously published list of HEG1 interacting proteins (26) was looked to and it was found that partitioning defective 3 homolog (PARD3) was such an interactor. Indeed, using the HEG1 matrix, at least three of the PARD3 isoforms were pulled down from HUVEC lysates, confirming that PARD3 binds to the HEG1 cytoplasmic tail (FIG. 5C). Importantly, PARD3 did not bind to the HEG1 ΔYF missing the last 2 C-terminal amino acids that are important for KRIT1 binding, suggesting that it binds to the same region of HEG1 as KRIT1. Finally, the addition of either HKi2 or 2-hydroxy-1-naphthoic acid (BL-0558) that does not block KRIT1 binding to HEG1 had no effects on PARD3 binding (FIG. 5D). Thus, HKi2 is specific at blocking KRIT1 binding to HEG1 and did not affect PARD3 binding that binds to the same region of HEG1.

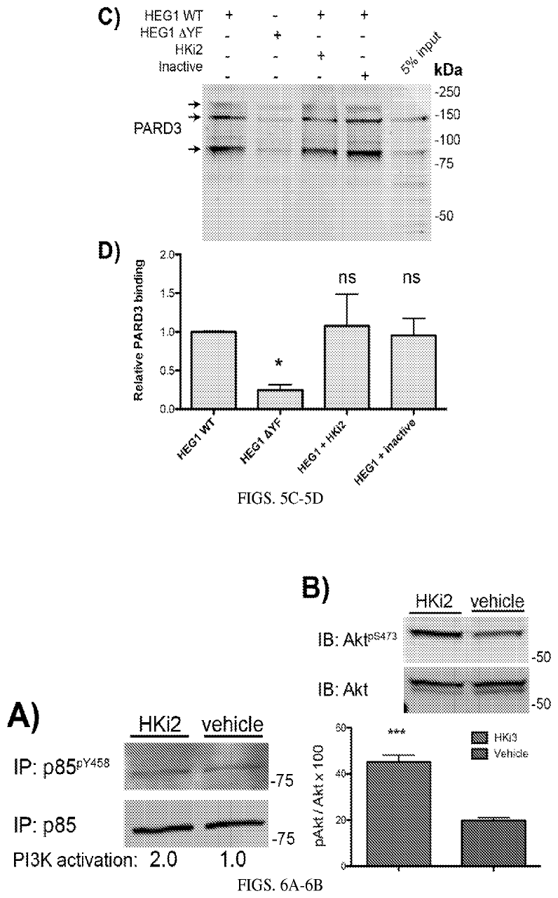

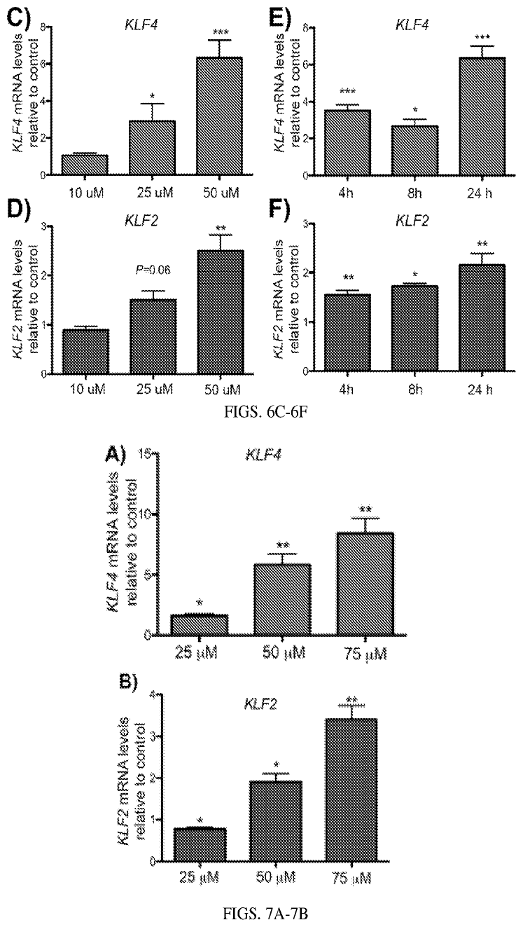

HKi2 increases PI3K/Akt activity and upregulates KLF4 and KLF2 levels in endothelial cells. To investigate the effects of acute inhibition of the endothelial HEG1-KRIT1 interaction, the human cerebral microvascular endothelial cell-line, hCMEC/D3, was used. The level of the phosphoinositide 3-kinase (PI3K)/Akt pathway implicated in the regulation of endothelial KLFs expression was assessed (20, 36, 37), hCMEC % D3 cells treated with 50 μM small molecule HKi2 for 1 h induced a 2-fold increase in PI3K activity (FIG. 6A). The increased PI3K activity also resulted in a 2.2-fold increase in Akt activation, as assessed by Western blot analysis of pAkt-S⁴⁷³ (FIG. 6B).

Genetic inactivation or knockdown of endothelial HEG1 or KRIT1 leads to the upregulation of endothelial KLF4 and KLF2 expression (12-16), but it was unknown whether disruption of the HEG1-KRIT1 interaction is sufficient to regulate endothelial KLFs. These results showed that indeed the KLF4 and KLF2 mRNA levels were upregulated following the addition of 25 μM HKi2 for 12 h of treatment (FIGS. 6C-6D). Increasing the concentration of HKi2 further upregulated KLF4 and KLF2 mRNA levels. While the upregulation of KLF4 mRNA levels (˜6.5 fold increase at 50 μM) was profound, when compared with controls (FIG. 6C), the changes in KLF2 mRNA levels (˜2.3 fold increase at 50 μM) were moderate but significant (FIG. 6D). It was also noted that incubation of hCMEC/D3 cells with 50 μM HKi2 induced a rapid upregulation of KLF4 (˜3.5 fid increase, FIG. 6E) and KLF2 (˜1.5 folds increase, FIG. 6F) as early as 4 h and continued to be increased until the end of the experiment at 24 h. Higher KLF4 levels (˜6 fold increase) remained to be detected following 24 h treatment (FIG. 6E). Importantly, hCMEC/D3 cells treated for 4 hours with 25 μM of a structurally-similar analog of HKi2 (2-hydroxy-1-naphthoic acid, FIG. 4), that failed to block HEG1-KRIT1 interaction, did not elevate KLF4 or KLF2, indicating that the effect of HKi2 is ascribable to the blockade of the HEG1-KRIT1 interaction. Thus, acute inhibition of the endothelial HEG1-KRIT1 interaction with HKi2 increases PI3K/Akt activity and is sufficient to elevate endothelial KLF4 and KLF2 expression.

Specifically, FIGS. 6A-6F show that HKi2 treatment activated PI3K/Akt signaling and leads to KLF2 and KLF4 upregulation in endothelial cells. (A-F) hCMEC/D3 cells treated with HKi2 or vehicle control and analyzed by Western blot for protein levels and by qPCR for mRNA levels. FIG. 6A shows that HKi2 treatment activated PI3K signaling as measured by phospho-p85. FIG. 6B shows that HKi2 treatment activated Akt signaling as measured by phosphor-Akt. FIGS. 6C-6D show dose response of KLF4 and KLF2 mRNA expression at indicated doses for 12 hours. HKi2 induces KLF4 and KLF2 mRNA expression at indicated concentrations. FIGS. 6E-6F show timecourse, HKi2 [50 μM] induces a rapid and sustained upregulation of KLF4 and KLF2 mRNA expression. In FIGS. 6B-6F, bar graphs represent protein or mRNA levels relative to vehicle control±SEM (n=3, 2-tailed t test). *, P<0.05; **, P<0.01; *** P<0.001.

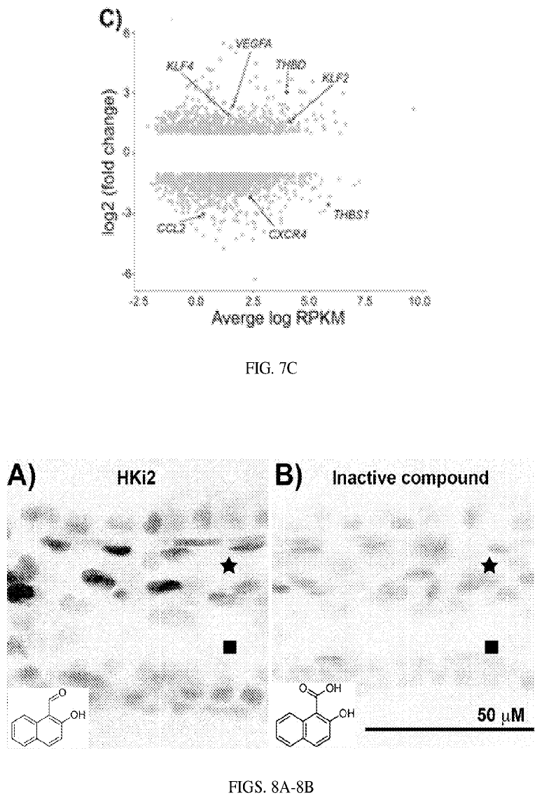

HKi2 upregulates KLF4 and KLF2 target genes in endothelial cells. Primary human umbilical vein endothelial cells (HUVEC) were used to study the effect of HKi2 on endothelial gene expression. Similar to hCMEC/D3 cells, HUVEC-treated with HKi2 upregulated both KLF4 and KLF2 mRNA levels in a dose-dependent manner (FIGS. 7A-7B). Genome-wide RNA sequencing (RNA-seq) was used to characterize further the effects of inhibition of the endothelial HEG1-KRIT1 interaction at the transcriptional level. Deep sequencing of cDNA from HUVEC after 24 h treatment with 75 μM HKi2 (FIG. 7C) revealed that disruption of the HEG1-KRIT1 protein interaction caused a dramatic change in the gene expression profile in endothelial cells. 457 genes differentially expressed between HKi2 treatment and vehicle control were identified (corrected P<0.05, ≥2.5-fold change). Among the most notable changes were KLF4 and KLF2 direct target genes including, upregulation of VEGFA (encoding vascular endothelial growth factor A, VEGF-A), and THBD (encoding thrombomodulin, TM) (FIG. 7C). Among the most notably downregulated were genes encoding receptors that regulate angiogenesis or secreted proteins, including THBS1 (encoding thrombospondin1, TSP1), CXCR4 (encoding C-X-C chemokine receptor type 4, CXCR-4), and CCL2 (encoding monocyte chemoattractant protein, MCP1) (FIG. 7C). Importantly, using the same conditions, two structurally similar compounds were tested that were shown to be inactive in blocking the HEG1-KRIT1 interaction in vitro, 2-hydroxy-1-naphthoic acid and naphthalene-2-ol (FIG. 4), and found no significant effects on gene expression by RNA-Seq (Data not shown). These results further confirm that the effects of HKi2 are ascribable to the blockade of the endothelial HEG1-KRIT1 interaction.

Specifically, FIGS. 7A-7C show HKi2 treatment leads to KLF4 and KLF2 upregulation, and their important transcriptional targets. In FIGS. 7A-7C, HUVEC was treated with HKi2 or vehicle control. FIGS. 7A-7B show dose response of KLF4 and KLF2 mRNA expression as determined by qPCR at indicated doses for 24 hours. HKi2 induces KLF4 and KLF2 mRNA expression at indicated concentrations. Bar graphs represent mRNA levels relative to vehicle control±SEM (n=3, 2-tailed/test). *, P<0.05; **, P<0.01; ***, P<0.001. FIG. 7C shows expression levels of differentially expressed genes upon HKi2 treatment [75 μM] represented on a scatter plot; reads per kilobase of transcript per million mapped reads (RPKM) of individual transcripts are represented on a log 2 scale. A few of the most highly suppressed and up-regulated genes are labeled.

HKi2 induces expression of klf2a in arterial and venous endothelium in zebrafish. The effect of acute inhibition of the HEG1-KRIT1 protein complex in vivo was addressed. Zebrafish embryos in which the KRIT1-HEG1 interaction is conserved were used (2, 7, 13), and which provide unique advantages of optical transparency that allow visualization of individual genes using non-invasive imaging (38). A transgenic klf2a reporter line, Tg(klf2a:H2B-EGFP), which consists of a 6-kb fragment of the klf2a zebrafish promoter driving the expression of the nuclear-localized histone-EGFP fusion protein was used (32, 33). The results showed that zebrafish embryos treated with 4 μM HKi2 for 4 h at 26 hours post-fertilization (hpf), displayed an increase of EGFP in the arterial and venous endothelium (FIG. 8A). Importantly, no effects on nuclear EGFP were observed in embryos treated with an inactive compound (FIG. 8B) or control vehicle DMSO (data not shown). These data show that blocking the HEG1-KRIT1 protein complex triggers an elevation of KLF2 expression in vivo.

Specifically, FIGS. 8A-8B show that HKi2 induces expression of klf2a in arterial and venous endothelium in zebrafish. Negative image of Tg(klf2a:H2b-EGFP) zebrafish embryos treated with: 4 μM HKi2 (FIG. 8A); or inactive control compound, 2-hydroxy-1-naphthoic acid (FIG. 8B). The compounds were added at 26 hpf, and images were taken at 30 hpf. The trunk vessels were scanned using Airyscan. Star and square indicate dorsal aorta and posterior cardinal vein, respectively. Lateral view with anterior to the bottom and dorsal to the top.

### DISCUSSION

HEG1 cytoplasmic tail binds directly to the KRIT1 FERM domain through discrete and unique interactions (5) and the loss of endothelial HEG1 or KRIT1 increases KLF4 and KLF2 gene expression (12-16). However, until this invention, the biological effect of inhibiting endothelial HEG1-KRIT1 interaction was incompletely understood due to the lack of tools to block their interaction while keeping their own integrity. In this invention, the pharmacological inhibition of the endothelial HEG1-KRIT1 interaction was evaluated as a new tool to identify downstream signaling pathways of the acute HEG1-KRIT1 protein complex disruption. A reliable and quantitative assay was developed to study the HEG1-KRIT1 protein interaction by flow cytometry. This approach led to the identification of a new class of small molecule HEG1-KRIT1 inhibitors now denominated HKi. X-ray co-crystal structure studies of KRIT1 FERM domain in complex with HKi1 and HKi2 demonstrate that the naphthalene fragment retained the same binding mode within the HEG1 binding pocket on KRIT1. Fragments of ligands that fully overlap with the strongest hot spot generally retain their position and binding mode when the rest of the molecule is removed (39). The low μM IC₅₀ values of these smaller fragments, especially HKi2, are considerably more potent (i.e., approximately ˜100-1000 times) than those typically observed for low MW fragments that can establish only a few non-covalent interactions with the target protein. This suggests that the relatively reactive carbonyl moiety of HKi2 may undergo covalent reversible binding with the KRIT1 FERM domain, as previously observed for peptidyl aldehydes inhibitors of Src homology 2 (SH2) domains (40). Among the 20 proteinic amino acids, the side chains of lysine and arginine are capable of forming covalent reversible interactions with aldehydes (typically in the form of an iminic or enamine adducts). Indeed, the crystal structure shows that the HEG1 binding pocket of KRIT1 contains three lysines residues positioned to engage the aldehyde of HKi2 in covalent reversible binding. Therefore, the reversible nature of covalent bond formation produces a relatively potent inhibition of the HEG1-KRIT1 interaction with IC₅₀ values in the low μM range. Therefore, HKi2 is a bona fide inhibitor of the HEG1-KRIT1 interaction. In addition, the relatively small size and reduced lipophilicity of HKi2 makes it a good starting point for future optimizations using fragment-based drug design (41).

Pharmacological inhibition of the HEG1-KRIT1 protein interaction can be used to study the signaling events regulated by this protein complex. Indeed, acute inhibition of the endothelial HEG1-KRIT1 interaction with HKi2 rapidly increases PI3K/Akt activity. Previous studies have shown that mechanotransduction via fluid shear stress mediates PI3K/Akt activation (22, 42, 43). Mechanistically, fluid shear stress increases tension on PECAM1 and subsequent activation of Src family kinases-induced ligand-independent VEGFR2/3 activation that, in turn, activates PI3K (22, 42, 44). However, the molecular connection between flow-induced mechanotransduction and cerebral cavernous malformation (CCM) proteins (e.g HEG1-KRIT1 protein complex) is still unclear (44). Rap1 has been proposed to be activated by laminar shear stress to promote the endothelial mechanosensing protein complex by increasing the association between PECAM1-VEGFR2-VE-cadherin and subsequent PI3K/Akt signaling (45). Importantly, Rap1 activity regulates the junctional localization of KRIT1 (4), and previous crystal structure analysis revealed that HEG1-KRIT1-Rap1 can form a ternary complex (3). This invention shows that there is no competition between HEG1 binding and Rap1 binding to the KRIT1 FERM domain, and it is not expected that HKi2 binding would affect Rap1 binding either. In fact, in this invention, the KRIT1-Rap1 complex was crystallized in the presence of HKi's because they diffract better than the KRIT1 FERM alone, supporting that HKi's do not affect Rap1 binding to KRIT1.

This invention demonstrates that pharmacological inhibition of the endothelial HEG1-KRIT1 interaction is sufficient to increase KLF4 and KLF2 expression in a dose- and time-dependent manner. It is well documented that genetic inactivation or knockdown of endothelial HEG1 or KRIT1 results in upregulation of KLF4 and KLF2, which are genes normally induced by laminar blood flow (12-16, 18, 19). Importantly, the gain of endothelial MEKK3 activity has been associated with the upregulation of KLF4 and KLF2 in the CCM disease (12-14). MEKK3 interacts with the CCM protein complex by binding directly to CCM2 (17, 46), and loss of CCM proteins results in an increase in MEK5-ERK5-MEF2 mechanotransduction pathway (12-14, 18, 19, 46) that may contribute to the responsiveness of endothelial cells to laminar blood flow (7). In agreement with these findings, inhibition of the HEG1-KRIT1 interaction by HKi2 mimics many of the transcriptional effects of laminar blood flow on the endothelium, including increased expression of genes that encode anticoagulants (e.g., THBD) and suppressed expression of genes that antagonize angiogenesis (e.g., THBS1) and NFκB-driven proinflammatory genes (e.g., CCL2). Therefore, the HEG1-KRIT1 protein complex is interconnected to mechanosensing proteins (e.g., PECAM1, VE-cadherin, and VEGFR2/3) that respond to flow-induced mechanotransduction (22, 23). Thus, novel HKi will provide new tools for analysis of the signaling events that follow disruption of HEG1-KRIT1 interaction with previously inaccessible temporal precision. Moreover, HKi may also be used in the treatment of cardiovascular diseases.

Cardiovascular diseases are currently the main cause of death in the world (20) and morbidity is usually due to thrombosis. Under normal circumstances, vascular endothelial cells exhibit anticoagulant, fibrinolytic and anti-inflammatory properties that limit thrombosis (21, 22). These thromboresistant properties of endothelial cells are enhanced by laminar blood flow that regulates multiple molecular mechanisms including the synthesis of vasoactive, anti-inflammatory and anti-thrombotic molecules (21, 23). Loss of these endothelial functions is associated with increased cardiovascular morbidity (22-24). Therefore, therapeutic strategies can be developed to support endothelial vasoprotection (21, 22, 25, 26). Many of the vasoprotective effects of laminar blood flow are due to upregulation of transcription factors KLF2 and, which in turn can increase expression of genes that encode anticoagulants (e.g. THBD encoding thrombomodulin, TM) or vasodilators (e.g. NOS3 encoding endothelial nitric oxide synthase, eNOS), and suppress expression of genes that antagonize angiogenesis (e.g. THBD encoding thrombospondin1, TSP1) and NFκB-driven proinflammatory genes (e.g. vascular adhesion molecules including, VCAM1 and ICAM1). Thus, laminar flow can upregulate KLF2 and KLF4 in endothelium to antagonize inflammation, atherosclerosis and thrombosis (9, 22, 24, 26-29).

Loss of KRIT1 leads to cerebral cavernous malformations (CCM) (30). That said, there is abundant evidence from murine models that perinatal endothelial-specific inactivation of Krit1 leads to CCM (6, 9, 31), whereas genetic inactivation of endothelial Krit1 in adults does not. Moreover, HEG1 mutations have never been identified in human CCM and deletion of HEG1 in mice does not cause CCM (1). This invention demonstrates the impact of pharmacological inhibition of the HEG1-KRIT1 protein complex which upregulates KLF2 and KLF4, and therefore attenuate proinflammatory and pro-thrombotic responses of EC to inflammatory cytokines. Thus, small molecules, including the novel HKi's, that disrupt the HEG1-KRIT1 interaction mimic the effect of laminar blood flow which induces an array of vasoprotective genes.

The hit compound (HKi001) identified in the primary screen, Sirtinol, is a class III Histone/Protein deacetylase (sirtuin) inhibitor. Sirtuins are structurally and mechanistically distinct from other classes of histone deacetylases (HDAC). They have been implicated in influencing a wide range of cellular processes like aging, transcription, apoptosis, inflammation and stress resistance, as well as energy efficiency and alertness during low-calorie situations. To distinguish the known activity of sirtinol on sirtuins from the HEG1-KRIT1 inhibition activity, the crystal structure of KRIT1 bound to sirtinol was examined and the aldehyde was identified as the active moiety. 2-hydroxy-1-naphthaldehyde (HKi2) alone also inhibited the HEG1-KRIT1 interaction and with better IC₅₀ values of 3.5 μM. Thus the naphthaldehyde group makes important contacts with the HEG1 binding pocket on KRIT1, as evidenced by the crystal structure, and is primarily responsible for the inhibitory activity of the molecule. The low μM IC50 of this compound is about 100-1000 times more potent than typical fragments described in published fragment-based drug discovery (FBDD) programs. In addition the X-ray structure shows that the carbonyl is no longer co-planar with the aromatic ring. Thus this suggests that the inhibitor underwent covalent interaction with the KRIT1 FERM domain. It has previously been reported that aldehydes can be reversible covalent inhibitors of Src homology 2 (SH2) domains. Those results were consistent with the formation of a reversible imine adduct between their compound and an amino group of the SH2 domain. However, in this invention, there was no observation of the covalent adduct directly, probably due to the reversible nature of the adduct and the moderate affinity. Among the 20 proteinic amino acids, only lysine and arginine are capable of forming such a structure with an aldehyde (in the form of an iminic or enamine). The crystal structure shows that the HKi2 pocket of KRIT1 contains three lysine residues (K475, K724, and K720). Thus, the aldehyde of HKi2 functions as covalent but reversible inhibitor of the HEG1-KRIT1 interaction.

This new compound allows blockade of the protein complex by specifically blocking the interaction between HEG1 and KRIT1 proteins, leaving the other functions or those proteins intact in contrast to the current approaches that compromise protein synthesis. Given that HKi2 can penetrate cells, it is expected that it can enter human cells in vivo. Furthermore, because both the KRIT1 FERM domain and the HEG1 cytoplasmic tails are highly conserved from zebrafish to humans, it is expected that HKi2 will be used in many systems.

Specific inhibitors of the HEG1-KRIT1 interaction were generated by modifying the basic structure of the lead compound, Hki2. The generated compounds are listed in Table 2. While a few of the compounds are commercially available, the majority of the compounds are novel and newly synthesized. NI means no inhibition was detected. Compounds with IC50 of less than 500 μM act as inhibitors.

TABLE 2

| Compound | Chemical name | IC50 (μM) |
|---|---|---|
| Sirtinol (Hki1) | 2-[[(2-hydroxy-1-naphthalenyl)methylene] | ~10 |
| | amino]-N-(1-phenylethyl)-benzamide | |
| BL-0547 (Hki3) | (E)-2-(((2-hydroxynaphthalen-1- | 2.9 |
| | yl)methylene)amino)benzoic acid | |
| 2-hydroxy-1- | 2-hydroxy-1-naphthaldehyde | 3.5 |
| naphtaldehyde | | |
| (Hki2) | | |
| Compound 4 | 2-amino-N-(1-phenylethyl)benzamide | NI |
| Compound 5 | 2-(1-aminoethyl)aniline | NI |
| BL-0549 | (E)-1-(((1H-tetrazol-5-yl)imino)methyl)naphthalen-2-ol | 3.2 |
| BL-0558 | (E)-3-(((2-hydroxynaphthalen-1- | 3.2 |
| | yl)methylene)amino)benzoic acid | |
| BL-0552 | (E)-4-(((2-hydroxynaphthalen-1- | 1.8 |
| | yl)methylene)amino)benzoic acid | |
| BL-0550 | (E)-1-(((1H-1,2,4-triazol-5-yl)imino) | precipitation |
| | methy)naphthalen-2-ol | |
| BL-0551 | (E)-1-((pyridin-3-ylimino)methyl)naphthalen-2-ol | precipitation |
| BL-0588 | 2-(((2-hydroxynaphthalen-1-yl)methyl)amino)benzoic acid | NI |
| BL-0593 | naphtho[1,2-d]isoxazole | NI |
| BL-0589 | methyl 2-(2-hydroxy-1-naphthamido)benzoate | precipitation |
| BL-0590 | 2-(2-hydroxy-1-naphthamido)benzoic acid | NI |
| BL-0591 | 2-hydroxy-l-naphthaldehyde oxime | NI |
| BL-0592 | 1-(aminomethyl)naphthalen-2-ol | NI |
| BL-0604 | 2-((2-hydroxynaphthalen-1-yl)ethynyl)benzoic acid | NI |
| BL-0605 | (E)-2-(2-(2-hydroxynaphthalen-1-yl)vinyl)benzoic acid | NI |
| BL-0606 | 2-(2-(2-hydroxynaphthalen-1-yl)ethyl)benzoic acid | NI |
| BL-0607 | Isoquinoline-1-carbaldehyde | NI |
| BL-0628 (HKi4) | 4-Hydroxy-[1,1′-biphenyl]-3-carbaldehyde | 21.8 |
| BL-0661 (HKi7) | 1-((phenylimino)methyl)naphthalene-2,6-diol | 32 |
| BL-0666 (HKi8) | 2-hydroxy-6-methyl-1-naphthaldehyde | 5.1 |
| BL-0670 (HKi6) | 2-hydroxy-6-methoxy-1-naphthaldehyde | 15 |
| BL-0691 | 2-hydroxy-8-methoxy-1-naphthaldehyde | 18 |
| BL-0693 | 2,8-dihydroxy-1-naphthaldehyde | 45 |
| BL-0695 | 3-(naphthalen-1-yl)oxetan-3-ol | NI |
| BL-0700 (HKi9) | 6-acetyl-2-hydroxy-1-naphthaldehyde | 58.6 |
| BL-702 (Hki10) | 6-chloro-2-hydroxy-1-naphthaldehyde | 3.2 |
| BL-703 | 2-(2-hydroxynaphthalen-1-yl)propane-1,3-diol | NI |
| BL-704 | 1-(Methylsulfonyl)naphthalen-2-ol | NI |
| BL-705 | 1-(Methylsulfinyl)naphthalen-2-ol | NI |
| BL-706 | 1-(2,2,2-Trifluoro-1-hydroxyethyl)naphthalen-2-ol | NI |
| BL-707 | 1-(methylthio)naphthalen-2-ol | NI |
| BL-708 | 2,2,2-trifluoro-1-(2-hydroxynaphthalen-1-yl)ethan-1-one | NI |
| BL-0713 | 1-(1-hydroxyethyl)naphthalen-2-ol | NI |
| BL-0714 | 2,2,2-Trifluoro-1-(2-hydroxynaphthalen-1-yl)ethan-1-one | NI |
| Compound 7 | 1-naphthaldehyde | 75 |
| (HKi5) | | |
| Compound 9 | 2-hydroxy-1-naphthoic acid | NI |
| Compound 10 | naphthalene-2-ol | NI |
| Compound 16 | Salicylaldehyde | NI |
| BL-0736 | 2-Hydroxy-6-(piperidin-1-yl)-1-naphthaldehyde | 28.8 |
| BL-0737 | 6-(Dimethylamino)-2-hydroxy-1-naphthaldehyde | 2.6 |
| BL-0738 | 2-Hydroxy-6-(2-oxoazetidin-1-yl)-1-naphthaldehyde | 30.3 |
| BL-0739 | 6-Allyl-2-hydroxy-1-naphthaldehyde | 1.1 |
| BL-0740 | 6-Ethyl-2-hydroxy-1-naphthaldehyde | 3.9 |
| BL-0742 | 6-Ethynyl-2-hydroxy-1-naphthaldehyde | 4 |
| BL-0743 | 6-Butyl-2-hydroxy-1-naphthaldehyde | 78.8 |
| BL-0744 | Ethyl (E)-3-(5-formyl-6-hydroxynaphthalen-2-yl)acrylate | 79.3 |
| BL-0745 | 2-Hydroxy-6-(1-hydroxyethyl)-1-naphthaldehyde | 31.9 |
| BL-0788 | 5-Formyl-6-hydroxy-2-naphthonitrile | 3.6 |
| BL-0794 | 6-Ethoxy-2-hydroxy-1-naphthaldehyde | 4.6 |
| BL-0817 | 2-Hydroxy-6-(prop-1-yn-1-yl)-1-naphthaldehyde | 15 |
| BL-0818 | 2-Hydraxy-6-phenyl-1-naphthaldehyde | 3 |
| BL-0819 | 2-Hydroxy-6-(thiophen-2-yl)-1-naphthaldehyde | 1.2 |

Several of the compounds may be even more effective than HKi2 due to their low IC50 values, such as HKi6, or other compounds with an IC50 below 3.5 μm. Structures of all compounds are shown in compound synthesis section.

Evaluation of a set of derivatives identified the 6-methoxy derivative (HKi6 or BL-0670) with an IC₅₀ of 1.5 μM. Importantly, the crystal structure with HKi6 (not shown) confirmed that the methoxy group in position 6 is projecting towards an adjacent socket that was originally identified in the HKi2 structure. Furthermore, the crystal structure data reveal that the pendant methoxy group is establishing a H-bond with the backbone of Gln⁴⁷³. Taken together, these findings suggest that further growth and functionalization of HKi6 is likely to lead to derivatives with improved complementarity and inhibition activity.

Importantly, KLF2/4 differentially regulates the expression of factors that confer anti-inflammatory, antithrombotic, and antiproliferative effects in ECs. In this invention, pharmacological inhibition of the HEG1 and KRIT1 interaction upregulates the gene expression levels of the transcription factors KLF2 and KLF4 (KLF24), and therefore can be used to modulate the sensitivity of ECs to hemodynamic forces. Similarly, it has been shown that Statins can upregulate KLF2/4 gene expression and here a new pathway was identified to upregulate those two transcription factors. Thus, the data suggests that HKis could work like statins and offer a new pathway to upregulate KLF2/4 gene expression that could function through a different set of affected genes to mediate anti-thrombotic effects.

The compounds identified in this invention have beneficial clinical uses. Genome-wide RNA transcriptome analysis of HKi2-treated human ECs under static conditions revealed that, in addition to elevating KLF4/2, inhibition of the HEG1-KRIT1 interaction mimics many of the transcriptional effects of pulsatile shear stress (PSS). These results suggest that the positive effects of PSS on the endothelium can be partially mimicked by HEG1-KRIT1 inhibition through KLF4/2 upregulation. Therefore, vasoprotection can be achieved by pharmacological disruption of the HEG1-KRIT1 complex in the endothelium, via the elevation of KLF4/2.

Table 3 shows changes in potential vasoprotective gene expression following pharmacological inhibition of KRIT1-HEG1 protein interaction as determined by RNA-Seq. Data are ratios of experimental/control Fragments per Kilobase per Million Mapped reads for each indicated transcript (n=3). For more details about these methods see FIG. 7C.

TABLE 3

| Gene symbol | Protein symbol | Protein name | HKi2 treatment |
|---|---|---|---|
| KLF2 | KLF2 | Kruppel like factor 2 | 3.07 |
| KLF4 | KLF4 | Kruppel like factor 4 | 3.89 |
| THBD | TM | Thrombomodulin | 8.34 |
| THBSI | TSP1 | Thrombospondin 1 | −5.78 |
| CCL2 | MCP1 | Monocyte | −7.95 |
| | | chemoattractant | |
| | | protein | |
| CXCR4 | CXCR-4 | C-X-C chemokine | −4.41 |
| | | receptor type 4 | |

This set of genes are known to strongly reduce the contribution of the vascular endothelium to inflammation, thrombosis, and atherosclerosis. Thus, these compounds may be used to inflammatory diseases, including, but not limited to, rheumatoid arthritis, gout, spondyloarthritis, vasculitis (including polyarteritis nodosoa, granulomatosus with polyangitis, other ANCA+vasculitis, Takayasu's disease, and giant cell arteritis), adult respiratory distress syndrome, post-perfusion injury, glomerulonephritis, and cytokine storm. The compounds may also be used to treat thrombosis, including but not limited to, myocardial infarction, stroke, deep vein thrombosis, pulmonary embolus, thrombotic thrombocytopenic purpura, and COVID-19. The compounds may also be used to treat atherosclerosis, including, but not limited to, coronary artery disease, carotid atherosclerosis, cerebrovascular disease, vascular dementia, and aortic aneurysm.

In conclusion, these compounds represent a new line of therapeutics through a new signaling pathway that affects blood flow sensing and upregulates genes that have good properties. A screen was designed and an inhibitor of the KRIT1-HEG1 interaction was found. It was also found that inhibition of this signaling pathway can upregulate the transcription factors KLF2/4 that have anti-inflammatory properties that are predicted to be beneficial in diseases such as atherosclerosis. Importantly, disruption of the HEG1-KRIT1 interaction in a mature vascular bed will not lead to the formation of cerebral cavernous malformations (CCMs), which is only observed in early development or in a chronic process, but not in an acute setting such as with inhibitors. This pharmacological and genetic manipulation of the HEG1-KRIT1 mainly upregulates KLF4 in contrast to other pharmacological approaches such statins which preferentially upregulates KLF2 (25). The combination of the two approaches could complement each others in future therapeutics.

### Materials and Methods

All reagents were from Sigma (St Louis, Mo.) unless otherwise indicated. Plasticware was from VWR (Radnor, Pa.) and Greiner Bio-One (Monroe, N.C.). Neutravidin Bead sets for were from Spherotech, Inc., (Lake Forest, Ill.). All solutions were prepared with ultra-pure 18 MG water or anhydrous DMSO. Flow cytometric calibration beads were from Bangs Laboratories Inc., (Fishers, Ind.) and Spherotech, Inc. Off patent commercial libraries were purchased from Prestwick Chemical (Illkirch-Graffenstaden, France), SelleckChem (Houston, Tex.), Spectrum Chemical (New Brunswick, N.J.), and Tocris Bio-Science (Bristol, UK). A collection of on patent drugs from MedChem Express was also purchased (Monmouth Junction, N.J.) that was specifically assembled by UNM collaborators. All purchased libraries were provided as 10 mM stock solutions in 96-well matrix plates except the MedChem Express library which was provided as individual powders that were subsequently solubilized in DMSO. All libraries were reformatted using a Biomek FX^{P} laboratory automated workstation into 384-well plates for storage (Greiner A784201; Labcyte #PP-0200). Low volume dispensing plates (Labcyte #LP-0200) were assembled using an Agilent BioCell work station (Santa Clara, Calif.). Low volume dispensing plates (Labcyte #LP-0200) were assembled using an Agilent BioCell work station (Santa Clara, Calif.). The following compounds were purchased from: Sirtinol (Selleckchem); 2-hydroxy-1-naphthaldehyde (Ark Pharm); and 2-amino-N-(1-phenylethyl)benzamide (Enamine).

Plasmid construction and protein purification. HEG1 intracellular tail model protein was prepared as previously described (5). In brief. His6-tagged HEG1 intracellular tail containing an in vivo biotinylation peptide tag at the N-terminus was cloned into pET15b, expressed in BL21 Star (DE3) and purified by nickel-affinity chromatography under denaturing conditions. Synthetic human non-biotinylated HEG1 7-mer peptide (residues 1375-1381) was purchased from GenScript. His6-EGFP-KRIT1 (WT) FERM domain (417-736) and KRIT1 (L717,721A) mutant were cloned into pETM-11 and expressed in BL21 Star (DE3). Recombinant His-EGFP-KRIT1 was purified by nickel-affinity chromatography, and further purified by Superdex-75 (261600) size-exclusion chromatography (GE Healthcare). The protein concentration was assessed using the A280 extinction coefficient of 71,740 M⁻¹.

Human KRIT1 FERM domain, residues 417-736 was expressed and purified as described previously (3). Briefly, KRIT1 was cloned into the expression vector pLEICS-07 (Protex, Leicester, UK) and expressed in Escherichia coli BL21 Star (DE3) (Invitrogen). Recombinant His-tagged KRIT1 was purified by nickel-affinity chromatography; the His tag was removed by cleavage with tobacco etch virus protease overnight, and the protein was further purified by Superdex-75 (26/600) size-exclusion chromatography. The protein concentration was assessed using the A280 extinction coefficient of 45,090 M⁻¹.

Human Rap1 isoform Rap1b (residues 1-167) cloned into pTAC vector in the E. coli strain CK600K was the generous gift of Professor Alfred Wittinghofer (Max Planck Institute of Molecular Physiology, Germany). The Rap1b was expressed and purified as described previously (24). The protein concentration was assessed using a molar absorption coefficient of A280=19,480 M⁻¹ as previously reported (25).

Equimolar concentrations of KRIT1 FERM domain and GMP-PNP loaded Rap1b were mixed and loaded on a Superdex-75 (26/600). The column was pre-equilibrated and run with 20 mM Tris, 50 mM NaCl, 3 mM MgCl₂, and 2 mM DTT (pH 8). The final complex concentration was determined using a molar absorption coefficient of A280=61,310 M−1 for the KRIT1-Rap1b complex.

Bead Coupling. SPHERO Neutravidin Polystyrene Particles, 6-8 μM (Spherotech) were washed twice with wash buffer (20 mM Tris, 150 mM NaCl. pH 7.4 containing 0.01% NP-40, and 1 mM EDTA). Prior to incubation with biotin-tagged HEG 1 cytoplasmic tail protein, an appropriate volume of bead slurry was passivated to inhibit non-specific binding by incubation for 30 minutes at room temperature in reaction buffer containing 0.1% BSA (20 mM Tris, 150 mM NaCl, pH 7.4 0.01% NP-40, 1 mM EDTA, 1 mM DTT, and 0.1% BSA). Passivated beads were collected by centrifugation, resuspended to 3,600 particles/μl in reaction buffer and biotinylated HEG1 tail was added to a final concentration of 150 nM and incubated overnight on a rotator at 4° C. The beads were washed three times by centrifugation with ice-cold reaction buffer to remove unbound HEG1 peptide. Beads were diluted such that a final concentration of 2000 beads/μL were available for addition to assay plates.

PARD3 pulldown assay. Neutravidin agarose beads (Thermo Fisher) matrix with wild-type HEG1 cytoplasmic tail (1274-1381) and ΔYF (1274-1379) were previously described (5, 26). HUVEC were collected in cold lysis buffer (50 mM Tris-HCl, pH 7.4, 100 mM NaCl, 5 mM MgCl₂, 0.5% NP-40) plus protease inhibitor cocktail (Roche). A total of 20 μl of HEG1 matrix was added to 600 μg of clarified lysates and incubated at 4° C. overnight while rotating. All conditions contained either vehicle DMSO, 35 μM HKi2 or 35 μM 2-hydroxy-1-naphthoic acid. After three washes with cold lysis buffer, beads were mixed with sample buffer and proteins were separated by SDS-PAGE. Bound PARD3 was detected by using polyclonal Rabbit anti-PARD3 (Millipore, 07-330) antibody.

Flow cytometry assay. A final volume of 100 μl containing 140 nM EGFP-KRIT1 FERM domain, with 10% DMSO or 10% compounds in DMSO, was incubated for 15 minutes at room temperature on a rotator. 100 μl of beads were added to the mixture for a final volume of 200 μl at 1,000 particles/μl with 70 nM EGFP-KRIT1 and incubated for 15 minutes at room temperature on a rotator. The control beads were: without KRIT1 (minimum signal); with KRIT1 (maximum signal); and with KRIT1 plus 2 μM HEG1 7-mer (positive blocking control). The EGFP fluorescence was measured using a BD Accuri flow cytometer. For screening purposes, the final volume of the reaction was scaled down to 10 μl and samples were processed as previously described (27). For FIGS. 1D-1F, 2B, and 3E, a representative experiment is shown of at the least 3 independent repeats.

Assay plate assembly. Plate assays were performed in 384-well microtiter plates (Greiner Bio-one, #784101). Reaction buffer. HEG1-coupled beads, and EGFP-KRIT-FERM constructs were added using a MultiFlo™ Microplate Dispenser (BioTek Instruments, Inc.). Compounds were added to single-point assay plates pre-loaded with reaction buffer using a Biomek^{NX} liquid handler (BeckmanCoulter) equipped with a 100 nL pintool (V & P Scientific, Inc.). Compound libraries were dispensed to a final concentration of 10 μM. An equal volume (10 nL) of DMSO was added to the vehicle control wells. Following the addition of library compounds, 5 μL of assay buffer was added and the plates were mixed before addition of 5 μL of the protein-coupled bead mixtures; Plates were protected from light and incubated on a rotator for 15 minutes at room temperature. Binding of EGFP-KRIT to HEG1 coupled beads was evaluated using an Accuri C6 flow cytometer.

Data Acquisition. Assay plates were sampled using the HyperCyt™ high throughput flow cytometry platform (Intellicyt; Albuquerque, N. Mex.). During sampling, the probe moves from well to well and samples 1-2 μL from each well pausing 0.4 sec in the air before sampling the next well. The resulting sample stream consisting of 384 separated samples is delivered to an Accuri C6 flow cytometer (BD Biosciences; San Jose, Calif.). Plate data are acquired as time-resolved files that are parsed by software-based well identification algorithms and merged with compound library files. Plate performance was validated using the Z-prime calculation (28).

Compounds that satisfied hit selection criteria in the primary screen were cherry-picked from compound storage plates and tested to confirm activity and determine potency. Dose response data points were fitted by Prism software (GraphPad Software Inc., San Diego, Calif.) using nonlinear least-squares regression in a sigmoidal dose-response model with variable slope, also known as the 4-parameter logistic equation. Curve fit statistics were used to determine the concentration of test compound that resulted in 50% of the maximal effect (EC50), the confidence interval of the EC50 estimate, the Hill slope, and the curve fit correlation coefficient.

Crystallization of the KRIT1-Rap1b-HKis complexes. The purified KRIT1 FERM domain-Rap1b complex at 8.25 mg/ml was used for crystallization. Crystals were grown at room temperature using the sitting-drop method by mixing equal volumes of protein complex and reservoir solution (2+2 μl). The reservoir solution contained 20-25% PEG 3,350, 100 mM Tris, pH 8.5, 100 mM KCl. After 1 week or later, ˜0.5 μl of 10 mM compounds in DMSO was added to the drop for 1 day. The crystals were briefly transferred to the reservoir solution containing 20% glycerol before freezing in liquid nitrogen.

Structure Determination. Diffraction data for the KRIT1 FERM domain-Rap 1b-HKis complexes were collected at the Advanced Light Source beamline 5.0.3. The data were processed with XDS (29). The structures were solved by molecular replacement using Phaser with the structure of the KRIT1-Rap1b complex (PDB ID: 4hdo). The model was then optimized using cycles of manual refinement with Coot and maximum likelihood refinement in Refmac5 as part of the CCP4 software suite (30). The small molecule inhibitors (HKi1 and HKi2) were built using coot Ligand Builder.

Cell culture. hCMEC/D3 cells at passages 30-37 were grown to confluence on collagen-coated plates and cultured using in EGM-2 MV medium and supplemented with complements obtained from the manufacturer (Lonza) as previously reported (31). HUVEC (Lonza) at passages 4-7 were grown to confluence on gelatin-coated plates and maintained using complete EGM-2 media (Lonza). HKi2, 10 mM in DMSO, was maintained at room temperature for 30 min rotating before use. Cells were then treated with HKi2 at the concentrations and times indicated for each experiment. Vehicle cells were treated with the same volume of DMSO as used with HKi2. Cells were maintained at 37° C. in 95% air and 5% CO₂.

Western blotting and immunoprecipitation. Following stimulation with 50 μM HKi2 or vehicle control (DMSO) for 1 hour, hCMEC/D3 cells were rapidly washed twice with ice cold PBS and lysed with lysis buffer (25 mM Tris, pH 7.5, 200 mM NaCl, 1% Triton X-100, 0.5% Sodium Deoxycholate, 2.5× protease inhibitor cocktail. 2.5×PhosSTOP). Cell lysates were spun at 20,000×g for 15 minutes at 4° C. The PI3 Kinase was immunoprecipitated using the mouse monoclonal antibody as described in the manufacturer instructions and supernatants were stored at −80° C. Samples were resolved on 4-12% gradient gel and blotted using specific antibodies, as indicated. Band intensity was determined using a Li-Cor system and values obtained for phosphoproteins were normalized to the total protein in the same sample.

Antibodies to phospho-Akt-Ser473 (clone: 193H12; rabbit mAb; #4058; 1:250), Akt (clone: 40D4; mouse mAb; #2920; 1:500), phospho-PI3 Kinase p85 Tyr458 (rabbit polyclonal; #4228; 1:500) were from Cell Signaling. Antibody to PI3 Kinase, p85 (clone AB6; mouse mAb; #05-212; 1:250) was from EMD Millipore.

RNA extraction and qRT-PCR. HUVECs total RNA were isolated using MagMAX™-96 for Microarrays Total RNA Isolation Kit, according to the manufacturer's protocol (Thermo Fisher Scientific Cat #AM1839). qPCR analysis, single-stranded cDNA was produced from 10 ng RNA isolated from HUVECs using PrimeScript™ RT Master Mix according to the manufacturer's protocol (Takara Cat. #RR036A). The levels of genes were analyzed using iTaq™ Universal SYBR Green (BioRad Cat #1725122) and thermal cycler (CFX96 Real-Time System; Bio-Rad) according to the manufacturer's protocol. Actin mRNA levels was used as internal control, and the 2^{-ΔΔCT} method was used for data analysis.

Genome-wide RNA sequencing. The quantity (ND-1000 spectrophotometer; NanoDrop Technologies) and quality (Bioanalyzer; Agilent) of total RNA were analyzed. Only RNA with a RNA integrity number (RIN) greater than 8 RNA was used for library preparation. Libraries were generated using Illumina's TruSeq Stranded mRNA Sample Prep kit using 400 ng RNA. RNA libraries were multiplexed and sequenced with 100-bp paired single-end reads (SR100) to a depth of 30 million reads per sample on an Illumina HiSeq2500. Fastq files from RNA-seq experiments were mapped to the human genome (GRCh primary assembly release 96) using Hisat2 with default parameters. All bioinformatics analyses were conducted in R using the systempipeR package RNAseq workflows. Differential gene expression analysis was conducted with EdgeR.

Zebrafish. A previously reported transgenic zebrafish line Tg(klf2a:H2B-EGFP) was used to monitor the expression of klf2a (32, 33). They embryos were treated at 26 hours post fertilization (hpf) with 4 μM of HKi2, or inactive compound (2-hydroxy-1-naphthoic acid), or vehicle DMSO for 4 hours. At 30 hpf, these treated embryos were scanned for EGFP expression by Zeiss LSM 880 Airiscan.

### Compound Synthesis

All solvents and reagents were reagent grade. All reagents were purchased from reputable vendors and used as received. Thin layer chromatography (TLC) was performed with 200 μM MilliporeSigma precoated silica gel aluminum sheets. TLC spots were visualized under UV light or using KMnO4 stain. Flash chromatography was performed with SilicaFlash P60 (particle size 40-63 μM) supplied by Silicycle. Proton and carbon NMR spectra were recorded on a 600 MHz NMR spectrometer. Chemical shifts were reported relative to residual solvent's peak. High-resolution mass spectra were measured at the University of California San Diego Molecular Mass Spectrometry Facility. All final compounds were found to be >95% as determined by HPLC/MS and NMR.

General procedure A. (imine synthesis): In a scaled tube is added 2-hydroxy-1-naphthaidehyde (1.00 equiv.), the corresponding amine (1.00 equiv.) and ethanol (1.00 mol/L). The reaction is heated at reflux for 2 h. At room temperature, the precipitate was filtered, washed with ethanol, diethyl ether and dry under vacuum to get the desired compound.

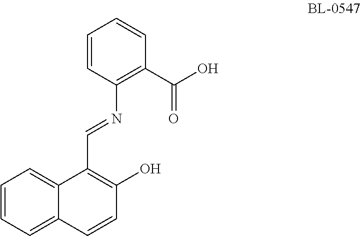 [SMILES: O=C(O)c1ccccc1/N=C/c1c(O)ccc2ccccc12]

(E)-2-(((2-hydroxynaphthalen-1-yl)methylene)amino)benzoic acid. The general procedure A was followed using 2-hydroxy-1-naphthaldehyde (300 mg, 1.74 mmol), 2-aminobenzoic acid (239 mg, 1.74 mmol) and ethanol (1.74 mL) to get the desired compound as a yellow solid in 97% yield (461 mg). ¹H NMR (600 MHz, DMSO) δ 15.13 (s, 1H), 13.49 (s, 1H), 9.36 (s, 1H), 8.38 (d, J=8.3 Hz, 1H), 8.03-7.95 (m, 2H), 7.84 (d, J=9.3 Hz, 1H), 7.73-7.68 (m, 2H), 7.49 (t, J=7.7 Hz, 1H), 7.35 (t, J=7.4 Hz, 1H), 7.30 (t. J=7.4 Hz, 1H), 6.79 (d, J=9.3 Hz, 1H) ppm. HRMS (ES+) calculated for C₁₈H₁₄NO₃ [M+H]⁺ 292.0968, found 292.0965. IR (neat) ν 1610, 1588, 1542, 1484, 1364, 1317, 1267, 1211, 1152, 1075, 972, 866, 838, 796, 758, 724, 599, 497, 475 cm⁻¹. As described in J. Am. Chem. Soc., 2015, 137 (1), pp 3958-396

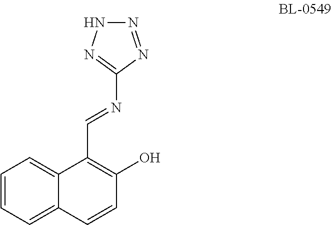 [SMILES: Oc1ccc2ccccc2c1/C=N/c1nn[nH]n1]

(E)-1-(((1H-tetrazol-5-yl)imino)methyl)naphthalen-2-ol. The general procedure A was followed using 2-hydroxy-1-naphthaldehyde (200 mg, 1.16 mmol). 5-aminotetrazole (99 mg, 1.16 mmol) and ethanol (1.16 mL) to get the desired compound as a yellow solid in 40% yield (111 mg). ¹H NMR (600 MHz, DMSO) δ 13.35 (s, 1H), 10.13 (s, 1H), 8.81 (d, J=8.5 Hz, 1H), 8.15 (d, J=9.1 Hz, 1H), 7.94 (d, J=7.9 Hz, 11H), 7.67 (t, J=7.7 Hz, 1H), 7.47 (t, J=7.4 Hz, 1H), 7.28 (d, J=9.1 Hz, 1H) ppm. HRMS (ES+) calculated for C₁₂H₁₀N₅O [M+H]⁺ 240.0880, found 240.0877. IR (neat) ν 1601, 1556, 1463, 1410, 1302, 1243, 1169, 1052, 828, 783, 744, 625, 523, 456 cm⁻¹. As described in Dalton Trans., 2014, 43, 6429-6435

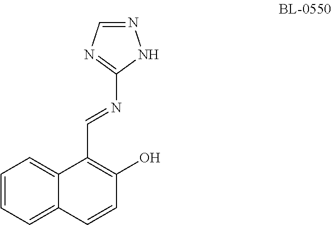 [SMILES: Oc1ccc2ccccc2c1/C=N/c1ncn[nH]1]

(E)-1-(((1H-1,2,4-triazol-5-yl)imino)methyl)naphthalen-2-ol. The general procedure A was followed using 2-hydroxy-1-naphthaldehyde (200 mg, 1.16 mmol), 3-amino-1,2,4-triazole (98 mg, 1.16 mmol) and ethanol (1.16 mL) to get the desired compound as a yellow solid in 79% yield (217 mg). ¹H NMR (600 MHz, DMSO) δ 14.82 (s, 1H), 14.21 (s, 1H), 10.08 (s, 1H), 8.54 (s, 1H), 8.42 (d, J=7.8 Hz, 1H), 8.04 (d, J=9.1 Hz, 1H), 7.88 (d, J=8.0 Hz, 1H), 7.61 (t, J=7.6 Hz, 1H), 7.42 (t, J=7.4 Hz, 1H), 7.19 (d, J=9.0 Hz, 1H) ppm. HRMS (ES+) calculated for C₁₃H₁₁N₄O [M+H]⁺ 239.0927, found 239.0928. IR (neat) ν 1623, 1604, 1572, 1524, 1474, 1451, 1427, 1305, 1244, 1190, 1167, 1086, 1012, 964, 815, 752, 634, 556 cm⁻¹.

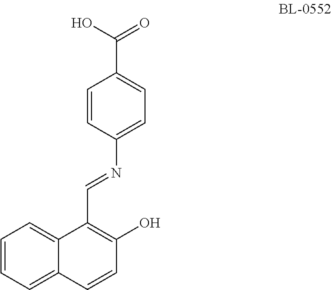 [SMILES: O=C(O)c1ccc(/N=C/c2c(O)ccc3ccccc23)cc1]

(E)-4-(((2-hydroxynaphthalen-1-yl)methylene)amino)benzoic acid. The general procedure A was followed using 2-hydroxy-1-naphthaldehyde (200 mg, 1.16 mmol), 4-aminobenzoic acid (159 mg, 1.16 mmol) and ethanol (1.16 mL) to get the desired compound as a yellow solid in 80% yield (272 mg). ¹H NMR (600 MHz, DMSO) δ 15.55 (s, 1H), 13.01 (s, 1H), 9.67 (s, 1H), 8.50 (d, J=8.4 Hz, 1H), 8.03 (d, J=8.4 Hz, 2H), 7.94 (d, J=9.2 Hz, 1H), 7.78 (d, J=7.9 Hz, 1H), 7.73 (d, J=8.4 Hz, 2H), 7.55 (t, J=7.6 Hz, 1H), 7.36 (t, J=7.4 Hz, 1H), 6.98 (d, J=9.3 Hz, 1H) ppm. ¹³C NMR (151 MHz, DMSO) δ 172.28, 166.87, 155.59, 147.26, 137.91, 133.23, 130.92, 129.13, 128.35, 128.16, 126.73, 123.82, 122.59, 120.55, 120.31, 108.75 ppm. HRMS (ES+) calculated for C₁₈H₁₄NO₃ [M+H]⁺ 292.0968, found 292.0972. IR (neat) ν 1677, 1624, 1579, 1542, 1432, 1283, 1210, 1150, 1117, 931, 852, 824, 768, 742, 688, 555, 492 cm⁻¹.

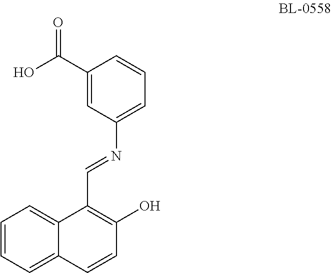 [SMILES: O=C(O)c1cccc(/N=C/c2c(O)ccc3ccccc23)c1]

(E)-3-(((2-hydroxynaphthalen-1-yl)methylene)amino)benzoic acid. The general procedure A was followed using 2-hydroxy-1-naphthaldehyde (200) mg, 1.16 mmol), 3-aminobenzoic acid (159 mg, 1.16 mmol) and ethanol (1.16 mL) to get the desired compound as a yellow solid in 90% yield (304 mg). ¹H NMR (600 MHz, DMSO) δ 15.66 (s, 1H), 13.24 (s, 1H), 9.72 (s, 1H), 8.53 (d, J=8.4 Hz, 1H), 8.07 (s, 1H), 7.94 (d, J=9.1 Hz, 1H), 7.92-7.86 (m, 2H), 7.79 (d, J=7.8 Hz, 1H), 7.61 (t, J=7.7 Hz, 1H), 7.54 (t, J=7.5 Hz, 1H), 7.35 (t, J=7.3 Hz, 1H), 7.04 (d, J=9.1 Hz, 1H) ppm. ¹³C NMR (151 MHz, DMSO) δ 169.71, 166.99, 157.04, 144.78, 137.00, 133.13, 132.35, 129.95, 129.05, 128.17, 127.19, 126.84, 124.92, 123.65, 121.82, 121.51, 120.66, 108.80 ppm. HRMS (ES+) calculated for C₁₈H₁₄NO₃ [M−H]⁺ 292.0968, found 292.0922. IR (neat) ν 1674, 1616, 1601, 1586, 1543, 1526, 1348, 1311, 1290, 1208, 1167, 1141, 1112, 897, 835, 751, 737, 722, 673, 646, 557, 500, 481 cm⁻¹.

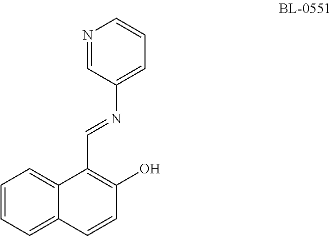 [SMILES: Oc1ccc2ccccc2c1/C=N/c1cccnc1]

H(E)-1-((pyridin-3-ylimino)methyl)naphthalen-2-ol. The general procedure A was followed using 2-hydroxy-1-naphthaldehyde (200 mg, 1.16 mmol), pyridin-3-amine (109 mg, 1.16 mmol) and ethanol (1.16 mL) to get the desired compound as a yellow solid in 73% yield (209 mg). ¹H NMR (600 MHz, DMSO) δ 15.32 (s, 1H), 9.77 (s, 1H), 8.80 (d, J=2.4 Hz, 1H), 8.56 (d, J=8.5 Hz, 1H), 8.51 (d, J=4.3 Hz, 1H), 8.11 (d, J=8.2 Hz, 1H), 8.00 (d, J=9.1 Hz, 1H), 7.85 (d, J=7.9 Hz, 1H), 7.58 (t, J=7.6 Hz, 1H), 7.53 (dd, J=8.1, 4.7 Hz, 1H), 7.39 (t, J=7.4 Hz, 1H), 7.11 (d, J=9.0 Hz, 1H) ppm. ¹³C NMR (151 MHz, DMSO) δ 167.36, 159.20, 147.45, 143.30, 141.89, 136.72, 132.90, 129.05, 128.20, 127.67, 127.07, 124.24, 123.79, 120.96, 120.81, 109.19 ppm. HRMS (ES+) calculated for C₁₆H₁₃N₂O [M+H]⁺ 249.1022, found 249.1020. IR (neat) ν 1298, 809, 747, 708, 621 cm⁻¹.

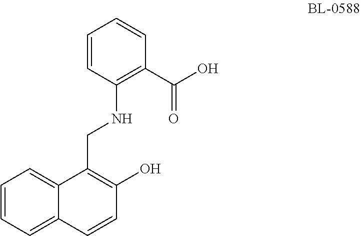 [SMILES: O=C(O)c1ccccc1NCc1c(O)ccc2ccccc12]

2-(((2-hydroxynaphthalen-1-yl)methyl)amino)benzoic acid. To a solution of (E)-2-(((2-hydroxynaphthalen-1-yl)methylene)amino)benzoic acid (50 mg, 0.17 mmol, 1.00 equiv.) in ethanol at 0° C. was portionwise added sodium borohydride (26 mg, 0.69 mmol, 4.00 equiv.). After the addition, the reaction was stirred at room temperature for 50 minutes. Water was added and the pH was adjust to 3 using HCl 1N. The reaction was extracted with EtOAc (×3). The combined organic layers were washed with brine, dried over Na₂SO₄, filtered and concentrated. The crude product was purified by silica gel column chromatography (5/95 MeOH/DCM) to give the desired compound as a yellow solid in 46% yield (23 mg). ¹H NMR (599 MHz, MeOD) δ 7.88 (d, J=8.1 Hz, 2H), 7.74 (d, J=8.1 Hz, 1H), 7.70 (d, J=8.9 Hz, 1H), 7.41 (t, J=7.7 Hz, 2H), 7.26 (t, J=7.4 Hz, 1H), 7.15 (d, J=8.9 Hz, 1H), 7.08 (d, J=8.4 Hz, 1H), 6.58 (t, J=7.5 Hz, 1H), 4.76 (s, 2H) ppm. ¹³C NMR (151 MHz, MeOD) δ 172.03, 154.36, 152.80, 135.70, 135.05, 133.17, 130.52, 130.34, 129.44, 127.68, 123.82, 123.77, 118.84, 116.62, 115.47, 112.69, 111.44, 38.42 ppm. HRMS (ES−) calculated for C18H14NO3 [M−H]⁻ 292.0979, found 292.0979. IR (neat) ν 3061, 1662, 1574, 1514, 1439, 1241, 1161, 813, 747 cm⁻¹.

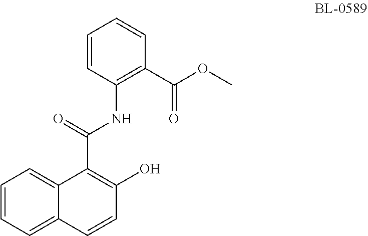 [SMILES: COC(=O)c1ccccc1NC(=O)c1c(O)ccc2ccccc12]

methyl 2-(2-hydroxy-1-naphthamido)benzoate. This procedure has been adapted from the following article: Molecules 2016, 21(8), 1068. In microwave tube was added 2-hydroxy-1-naphthaldehyde (50 mg, 0.27 mmol, 1.00 equiv.), methyl 2-aminobenzoate (40 mg, 0.27 mmol, 1.00 equiv.) and anhydrous toluene (1.50 mL). To this mixture was slowly added phosphorus trichloride (12 μL, 0.13 mmol, 0.50 equiv.). The reaction was heated at 130° C. in microwave for 15 minutes and then concentrated. The solid was washed with HCl 2N and ethanol to get the desired product as a beige powder in 42% yield (36 mg). ¹H NMR (600 MHz, DMSO) δ 11.31 (s, 1H), 10.47 (s, 1H), 8.75 (d, J=7.1 Hz, 1H), 7.99 (d, J=7.6 Hz, 1H), 7.93-7.85 (m, 3H), 7.72 (t, J=6.4 Hz, 1H), 7.48 (t, J=7.2 Hz, 1H), 7.35 (t, J=7.1 Hz, 11H), 7.24 (t, J=7.3 Hz, 1H), 3.79 (s, 3H) ppm. ¹³C NMR (151 MHz, DMSO) δ 167.75, 165.74, 152.29, 140.40, 134.46, 131.43, 131.39, 130.74, 128.19, 127.63, 127.33, 123.54, 123.31, 123.20, 120.44, 118.31, 117.07, 116.47, 52.52 ppm. HRMS (ES+) calculated for C₁₉H₁₅NNaO₄ [M+Na]⁺ 344.0899, found 344.0896. IR (neat) ν 1698, 1636, 1578, 1512, 1449, 1438, 1316, 1262, 1234, 1203, 1089, 964, 823, 795, 756, 727, 697, 508, 483 cm⁻¹.

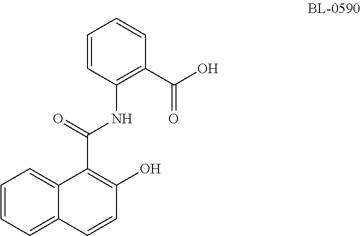 [SMILES: O=C(O)c1ccccc1NC(=O)c1c(O)ccc2ccccc12]

2-(2-hydroxy-1-naphthamido)benzoic acid. To a solution of methyl 2-(2-hydroxy-1-naphthamido)benzoate (50 mg, 0.16 mmol, 1.00 equiv) in methanol (0.8 mL) was added NaOH (2M, 0.4 mL). After 1.5 hour the reaction was concentrated to remove the methanol. HCl 3N was added until pH=1. The solution was extracted with EtOAc (3×) and the combined organic layers were dried over Na₂SO₄, filtered and concentrated to get the desired compound as a white solid in 67% yield (32 mg). ¹H NMR (600 MHz, DMSO) δ 13.56 (s, 1H), 11.66 (s, 1H), 10.43 (s, 1H), 8.89 (d, J=8.2 Hz, 1H), 8.04 (d, J=7.8 Hz, 1H), 7.91 (d, J=8.9 Hz, 1H), 7.89-7.83 (m, 2H), 7.70 (t, J=7.7 Hz, 1H), 7.47 (t, J=7.7 Hz, 1H), 7.35 (t, J=7.5 Hz, 1H), 7.27 (d, J=8.9 Hz, 1H), 7.22 (t, J=7.6 Hz, 1H) ppm. ¹³C NMR (151 MHz, DMSO) δ 169.43, 165.74, 152.21, 141.20, 134.39, 131.38, 131.32, 128.20, 127.60, 127.36, 123.50, 123.31, 122.88, 119.77, 118.33, 117.34, 116.20 ppm. HRMS (ES−) calculated for C₁₈H₁₂NO₄ [M−H]⁻ 306.0772, found 306.0773. IR (neat) ν 1711, 1679, 1637, 1604, 1578, 1537, 1405, 1327, 1246,815, 744,657 cm⁻¹.

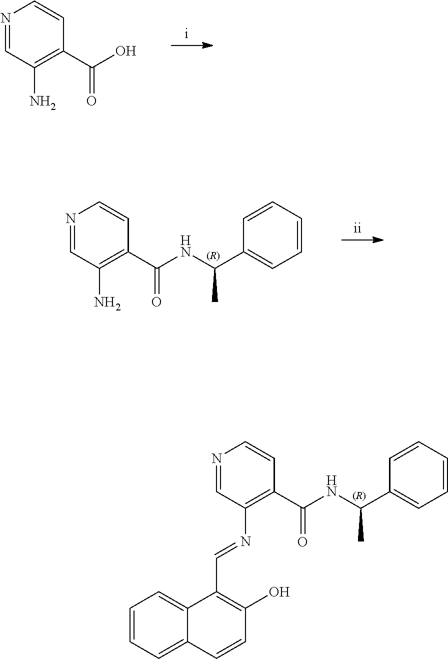

HBTU, Et₃N, (R)-1-phenylethan-1-amine, DCM, 16 h, rt, 82% ii) 2-hydroxy-1-naphthaldehyde, EtOH, 2 h, reflux, 33%.

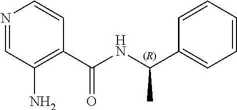

(R)-3-amino-N-(1-phenylethyl)isonicotinamide. To a solution of 3-aminoisonicotinic acid (200 mg, 1.45 mmol, 1.00 equiv.) in anhydrous dichloromethane (6.6 mL) was added HBTU (1.10 g, 2.90 mmol, 2.00 equiv), (R)-1-phenylethan-1-amine (186 μL, 1.45 mmol, 1.00 equiv.) and Et₃N (979 μL, 7.25 mmol, 5.00 equiv.). The reaction was stirred overnight and filtered. The filtrate was washed with water (2×), brine, dried over Na₂SO₄, filtered and concentrated. The crude product was purified by precipitation using a DCM/Hexanes (2/1) mixture. The solid was filtered, washed with pentane and dried under vacuum to give the desired product as a white solid in a 82% yield (288 mg). ¹H NMR (600 MHz, CDCl₃) δ 8.18-8.11 (m, 1H), 7.90 (d, J=5.1 Hz, 1H), 7.40-7.35 (m, 4H), 7.33-7.27 (m, 11H), 7.12 (d, J=5.2 Hz, 1H), 6.41 (d, J=7.1 Hz, 1H), 5.51 (s, 2H), 5.31-5.23 (m, 1H), 1.60 (d, J=6.9 Hz, 3H) ppm. ¹³C NMR (151 MHz, CDCl₃) δ 166.73, 143.77, 142.82, 140.81, 137.57, 129.01, 127.80, 126.27, 120.65, 119.82, 49.33, 21.91 ppm. HRMS (ES+) calculated for C₁₄H₁₆N₃O [M+H]⁺ 342.1288, found 242.1286 IR (neat) ν 3450, 3301, 1637, 1617, 1584, 1535, 1421, 1237, 841, 749, 702 cm⁻¹.

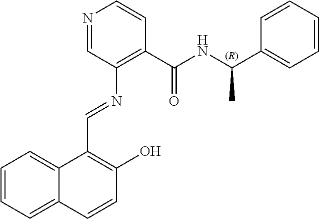

(R,E)-3-(((2-hydroxynaphthalen-1-yl)methylene)amino)-N-(1-phenylethyl)isonicotinamide. The general procedure A was followed using 2-hydroxy-1-naphthaldehyde (86 mg, 0.50 mmol), pyridin-3-amine (120 mg, 0.50 mmol) and ethanol (0.5 mL) to get the desired compound as a yellow solid in 33% yield (65 mg). ¹H NMR (600 MHz, DMSO) δ 9.69 (s, 114), 9.10 (s, 1H), 8.95 (s. II), 8.57 (s, 2H), 8.02 (d, J=6.3 Hz, 1H), 7.87 (s, 1H), 7.59-7.48 (m, 2H), 7.42-7.09 (m, 8H), 5.13 (s, 1H), 1.41 (s, 3H) ppm. ¹³C NMR (151 MHz, DMSO) δ 164.71, 160.05, 147.21, 144.09, 141.73, 139.81, 137.21, 136.59, 132.86, 129.05, 128.24, 127.20, 126.70, 125.99, 123.82, 121.62, 120.95, 120.62, 120.32, 118.80, 109.53, 48.46, 22.29 ppm. HRMS (ES+) calculated for C₂₅H₂₂N₃O₂ [M+H]⁺ 396.1707, found 396.1709. IR (neat) ν 1643, 1624, 1543, 1530, 1365, 1300, 1192, 833, 753, 699 cm⁻¹.

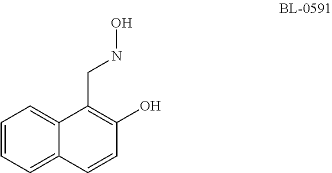 [SMILES: ONCc1c(O)ccc2ccccc12]

2-hydroxy-1-naphthaldehyde oxime. This procedure has been carried out according to the following article: Tetrahedron Letters 50 (2009) 6173-6175. To a solution of 2-hydroxy-1-naphthaldehyde (500 mg, 2.90 mmol, 1.00 equiv) in ethanol (21 mL) was added hydroxylamine hydrochloride. The reaction was heated at 65° C. for 18 h and then cooled to room temperature and poured in cold water. The product precipitated was collected by filtration. The crude product was purified by silica gel column chromatography (using 100% of DCM) to give the desired compound as a white solid in 63% yield (343 mg). ¹H NMR (600 MHz, CDCl₃) δ 10.84 (s, 1H), 9.14 (s, 1H), 7.96 (d, J=8.5 Hz, 1H), 7.80-7.75 (m, 2H), 7.52 (t, J=7.3 Hz, 1H), 7.36 (t, J=7.4 Hz, 1H), 7.25 (s, 1H), 7.20 (d, J=8.8 Hz, 1H) ppm. ¹³C NMR (151 MHz, CDCl₃) δ 157.52, 150.04, 132.74, 132.06, 129.16, 128.45, 127.67, 123.72, 120.35, 118.93, 106.89 ppm. HRMS (ES−) calculated for C₁₁H₈NO₂ [M−H]⁻ 186.0561, found 186.0560. IR (neat) ν 3323, 1633, 1591, 1464, 1414, 1308, 1269, 1241, 1182, 1016, 936, 814, 773, 743, 717, 646 cm⁻¹.

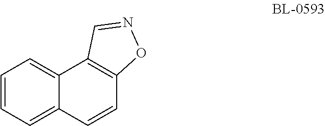 [SMILES: c1ccc2c(c1)ccc1oncc12]

naphtho[1,2-d]isoxazole. This procedure has been carried out according to the following article: Tetrahedron Letters 50 (2009) 6173-6175. To a solution of 2-hydroxy-1-naphthaldehyde oxime (50 mg, 0.27 mmol, 1.00 equiv) in anhydrous dichloromethane (6.6 mL) was added Et3N (93 μL, 0.69 mmol, 2.50 equiv) and then tosyl chloride (102 mg, 0.53 mmol, 2.00 equiv). The reaction was stirred 15 min and quenched with a 10% NaOH aqueous solution and separated. The organic layer was dried over MgSO₄, filtered and concentrated. The crude product was purified by silica gel column chromatography (0-50% of EtOAc in Hexanes) to give the desired compound as a pale yellow solid in 44% yield (20 mg). ¹H NMR (600 MHz, CDCl₃) δ 9.11 (s, 1H), 8.14 (d, J=8.1 Hz, 1H), 7.99 (d, J=8.1 Hz, 1H), 7.96 (d, J=9.0 Hz, 1H), 7.73 (d, J=9.1 Hz, 1H), 7.69 (t, J=7.5 Hz, 1H), 7.57 (t, J=7.5 Hz, 1H) ppm. ¹³C NMR (151 MHz, CDCl₃) δ 162.38, 145.07, 131.85, 130.56, 129.15, 128.31, 126.89, 125.72, 123.33, 116.51, 110.41 ppm. HRMS (ES−) calculated for C₁₁H₆NO [M−H]⁻ 168.0454, found 168.0464. IR (neat) ν1631, 1580, 1530, 1252, 1168, 930,844, 811,781, 753, 512, 461 cm⁻¹.

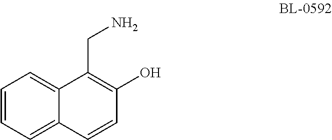 [SMILES: NCc1c(O)ccc2ccccc12]

1-(aminomethyl)naphthalen-2-ol To a solution of 2-hydroxy-1-naphthaldehyde oxime (70 mg, 0.37 mmol, 1.00 equiv) in acetic acid (2.10 mL) was added zinc dust (137 mg, 2.09 mmol, 5.60 equiv). The reaction was heated at 70° C. for 2 hours. The reaction was cooled to room temperature and filtered. The filtrate was concentrated and a 2M aqueous solution of NaOH was added until pH=8.5 and extracted with EtOAc (3×). The combined organic layers were washed with brine, dried over Na₂SO₄, filtered and concentrated. To the crude product was added dichloromethane. The precipitate was filtered and washed with pentane to get the desired product as a pink solid in 25% yield (16 mg). ¹H NMR (600 MHz, DMSO) δ 7.90 (d, J=7.7 Hz, 1H), 7.78 (d, J=7.2 Hz, 1H), 7.71 (d, J=8.3 Hz, 1H), 7.41 (s, 1H), 7.27 (s, 1H), 7.10 (d, J=8.2 Hz, 1H), 4.32 (s, 2H) ppm. ¹³C NMR (151 MHz, DMSO) δ 155.07, 133.01, 128.71, 128.39, 127.92, 126.29, 122.24, 122.12, 118.60, 114.59, 44.08 ppm. HRMS (ES−) calculated for C₁₁H₁₀NO [M−H]⁻ 172.0768, found 172.0768. IR (neat) ν1586, 1432, 1265, 1236, 812, 737, 537, 466 cm⁻¹.

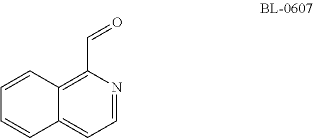 [SMILES: O=Cc1nccc2ccccc12]

isoquinoline-carbaldehyde. This procedure has been carried out according to the following article: Bioorg. Med. Chem. 20 (2012) 1201-1212. To a solution of 1-methylisoquinoline (200 mg, 1.40 mmol, 1.40 equiv) in anhydrous 1,4-dioxane was added selenium dioxide (217 mg, 1.96 mmol, 1.40 equiv). The reaction was heated at reflux for 90 minutes. The reaction was cooled to room temperature and filtered through a celite pad. The filtrate was concentrated and the residue was purified by silica gel column chromatography (0-15% of EtOAc in Hexanes) to give the desired compound as a pink solid in 61% yield (135 mg). ¹H NMR (600 MHz, CDCl₃) δ 10.33 (s, 1H), 9.31-9.18 (m, 1H), 8.68 (d, J=5.5 Hz, 1H), 7.85-7.79 (m, 2H), 7.72-7.65 (m, 2H) ppm. ¹³C NMR (151 MHz, CDCl₃) δ 195.66, 149.72, 142.44, 136.82, 130.77, 130.04, 126.96, 126.26, 125.66, 125.54 ppm. HRMS (ES+) calculated for C₁₉H₈NO [M+H]⁺ 158.0600, found 158.0601. IR (neat) ν 2832, 1702, 1579, 1453, 1386, 1320, 1205, 1142, 1055, 889, 834, 799, 714, 748, 655, 645, 469 cm⁻¹.

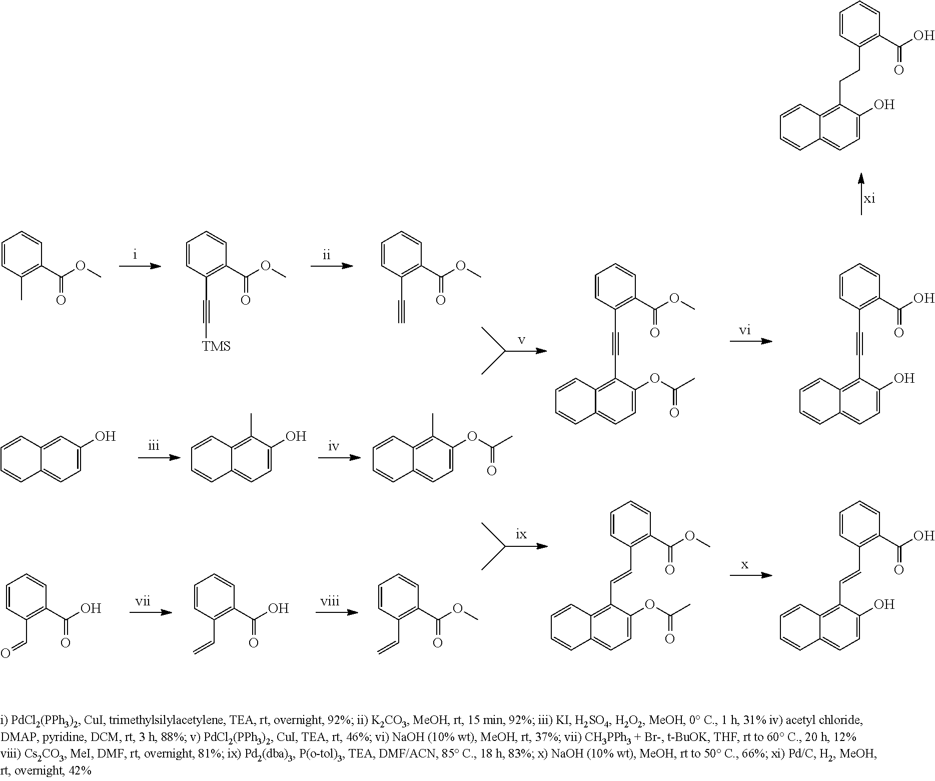

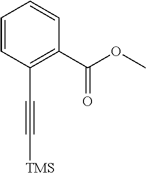 [SMILES: COC(=O)c1ccccc1C#C[Si](C)(C)C]

Methyl 2-((trimethylsilyl)ethynyl)benzoate. This procedure has been adapted from the following article: J. Org. Chem., 2009, 74 (3), pp 1141-1147. To a solution in methyl 2-iodobenzoate (1.00 g, 3.82 mmol, 1.00 equiv) in Et₃N (15.3 mL) was added Bis(triphenylphosphine)palladium chloride (53 mg, 0.08 mmol, 0.02 equiv) and copper(I) iodide (7 mg, 0.04 mmol, 0.01 equiv). The mixture was stirred at room temperature for 5 minutes. A solution of trimethylsilylacetylene (635 μL, 4.58 mmol, 1.20 equiv) in Et₃N (3.80 mL) was slowly added over 15 minutes. The reaction was flushed with nitrogen and stirred at room temperature overnight. The reaction was filtered through celite using Et₂O. The filtrate was washed with water, brine, dried over Na2SO4, filtered and concentrated. The residue was purified by silica gel column chromatography (0-5% of Et₂O in Hexanes) to give the desired compound as an orange oil in 92% yield (813 mg). ¹H NMR (599 MHz, CDCl₃) δ 7.90 (dd, J=7.9, 1.1 Hz, 1H), 7.58 (dd, J=7.7, 0.9 Hz, 1H), 7.44 (td, J=7.6, 1.3 Hz, 1H), 7.36 (td, J=7.6, 1.2 Hz, 1H), 3.92 (s, 3H), 0.27 (s, 9H) ppm. HRMS (ES+) calculated for C₁₃H₁₇O₂Si [M+H]⁺ 233.0992, found 233.0993. IR (neat) ν 2956, 2159, 1733, 1717, 1296, 1247, 1079, 865, 837, 755 cm⁻¹.

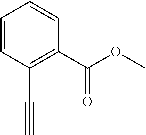

methyl 2-ethynylbenzoate. This procedure has been adapted from the following article: Org. Lett., 2010, 12 (16), pp 3651-3653. To a solution of methyl 2-((trimethylsilyl)ethynyl)benzoate (400 mg, 1.72 mmol, 1.00 equiv) in anhydrous MeOH (13.9 mL) was added anhydrous K₂CO₃ (238 mg, 1.72 mmol, 1.00 equiv). After 15 minutes, water was added. The mixture was extracted with EtOAc (3×). The combined organic layers were washed with brine, dried over Na₂SO₄, filtered and concentrated to give the desired compound as red liquid without further purification in 92% yield (253 mg). ¹H NMR (600 MHz, CDCl₃) δ 7.95 (dd, J=7.9, 1.0 Hz, 1H), 7.63 (d, J=7.3 Hz, 1H), 7.48 (td, J=7.6, 1.2 Hz, 1H), 7.41 (td, J=7.6, 1.0 Hz, 1H), 3.93 (s, 3H), 3.40 (s, 1H) ppm. HRMS (APCl+) calculated for C₁₀H₉O₂ [M+H]⁺ 161.0597, found 161.0600. IR (neat) ν 3282, 1722, 1433, 1294, 1274, 1253, 1129, 1078, 755, 660 cm⁻¹.

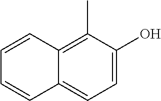

1-iodonaphthalen-2-ol. This procedure has been carried out according to the following article: Synthesis 2004, No. 11, 1869-1873. To a solution of H2SO4 (554 μL, 10.40 mmol, 1.50 equiv) in MeOH (35 mL) was added naphthalen-2-ol (1.00 g, 6.93 mmol, 1.00 equiv). The reaction was cooled at 0° C. K1 (1.15 g, 6.93 mmol, 1.00 equiv) and H₂O₂ (30% wt, 1.42 mL, 13.86 mmol, 2.00 equiv) were added. The reaction was stirred at 0° C. for 1 hour. DCM was added and the organic mixture was washed with aqueous solution of NaHSO3 (0.1M), water, brine, dried over Na2SO4, filtered and concentrated. The residue was purified by silica gel column chromatography (0-40% of DCM in Hexanes) to give the desired compound as grey solid in 31% yield (580 mg). ¹H NMR (600 MHz, CDCl₃) δ 7.93 (d, J=8.5 Hz, 1H), 7.77-7.72 (m, 2H), 7.55 (t, J=7.4 Hz, 1H), 7.39 (t, J=7.5 Hz, 1H), 7.26 (d, J=8.8 Hz, 1H), 5.79 (s, 1H) ppm. ¹³C NMR (151 MHz, CDCl₃) δ 153.86, 134.89, 130.76, 130.38, 129.78, 128.44, 128.35, 124.32, 116.57, 86.38 ppm. HRMS (ES−) calculated for C₁₀H₆IO [M−H]⁻ 268.9469, found 268.9467. IR (neat) ν 3292, 1624, 1497, 1430, 1345, 1301, 1237, 976, 924, 807, 744 cm⁻¹.

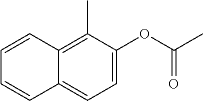

1-iodonaphthalen-2-yl acetate. To a solution of I-iodonaphthalen-2-ol (539 mg, 2.00 mmol, 1.00 equiv), DMAP (24 mg, 0.20 mmol, 0.10 equiv), pyridine (178 μL, 2.20 mmol, 1.10 equiv) in anhydrous DCM (7.30 mL) was slowly added acetyl chloride (170 μL, 2.39 mmol, 1.20 equiv). After 3 hours at room temperature, the reaction was quenched with a saturated solution of ammonium chloride. The mixture was extracted with DCM (3×). The combined organic layers were was with brine, dried over Na₂SO₄, filtered and concentrated. The residue was purified by silica gel column chromatography (1/9 EtOAc/Hexanes) to give the desired compound as pale yellow oil in 88% yield (547 mg). ¹H NMR (600 MHz, CDCl₃) δ 8.17 (d, J=8.5 Hz, 1H), 7.85 (d, J=8.7 Hz, 1H), 7.81 (d, J=8.1 Hz, 1H), 7.60 (t, J=7.5 Hz, 1H), 7.52 (t, J=7.4 Hz, 1H), 7.23 (d, J=8.7 Hz, 1H), 2.45 (s, 3H) ppm. ¹³C NMR (151 MHz, CDCl₃) δ 169.09, 150.16, 135.35, 132.25, 132.16, 130.32, 128.49, 128.38, 126.57, 121.52, 94.74, 21.56 ppm. HRMS (ES+) calculated for C₁₂H₉INaO₂ [M+Na]⁺ 334.9545, found 334.9538. IR (neat) ν 1765, 1366, 1184, 1011, 757 cm⁻¹.

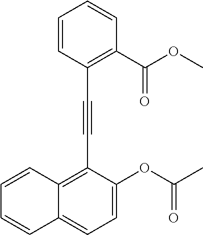 [SMILES: COC(=O)c1ccccc1C#Cc1c(OC(C)=O)ccc2ccccc12]

methyl 2-((2-acetoxynaphthalen-1-yl)ethynyl)benzoate. To a solution of 1-iodonaphthalen-2-yl acetate (411 mg, 1.32 mmol, 1.00 equiv) in Et3N (5.3 mL) was added Bis(triphenylphosphine)palladium chloride (19 mg, 0.03 mmol, 0.02 equiv) and copper(I) iodide (2.5 mg, 0.01 mmol, 0.01 equiv). The mixture was stirred at room temperature for 5 minutes. A solution of methyl 2-ethynylbenzoate (253 mg, 1.58 mmol, 1.20 equiv) in Et₃N (1.30 mL) was slowly added over 15 minutes. The reaction was flushed with nitrogen and stirred at room temperature for 24 hours. The reaction was filtered through celite using EtOAc. The filtrate was washed with water (×3), brine, dried over Na₂SO, filtered and concentrated. The residue was purified by silica gel column chromatography (0-20% of EtOAc in Hexanes) to give a mix of the desired compound and dimethyl 2,2′-(buta-1,3-diyne-1,4-diyl)dibenzoate (byproduct) in 70% yield (320 mg) with a purity of 65% (calculated by NMR) (208 mg). ¹H NMR (600 MHz, CDCl₃) δ 8.56 (d, J=8.3 Hz, 1H), 8.03 (dd, J=7.9, 1.1 Hz, 1H), 7.89-7.84 (m, 2H), 7.74 (dd, J=7.8, 1.0 Hz, 1H), 7.70-7.61 (m, 1H), 7.57-7.52 (m, 2H), 7.46-7.39 (m, 1H), 7.28 (d, J=8.8 Hz, 1H), 3.97 (s, 3H), 2.48 (s, 3H) ppm. HRMS (ES+) calculated for C₂₂H₁₇O₄ [M+H]⁺ 345.1121, found 345.1121. IR (neat) ν 1764, 1724, 1252, 1186, 1081, 754 cm⁻¹. Byproduct: dimethyl 2,2′-(buta-1,3-diyne-1,4-diyl)dibenzoate: ¹H NMR (600 MHz, CDCl₃) δ 8.00 (d, J=7.8 Hz, 2H), 7.68 (d, J=7.7 Hz, 2H), 7.50 (t, J=7.5 Hz, 2H), 7.43 (t, J=7.7 Hz, 2H), 3.97 (s, 6H) ppm (as described in Eur. J. Org. Chem. 2011, 238-242.). ¹³C NMR (151 MHz, CDCl₃) δ 166.14, 135.24, 132.68, 131.92, 130.66, 128.83, 122.56, 81.55, 78.97, 77.16, 52.45, 52.41 ppm. HRMS (ES+) calculated for C₂₀H₁₅O₄ [M+H]⁺ 319.0965, found 319.0970. IR (neat) ν 2950, 1718, 1479, 1432, 1292, 1271, 1252, 1198, 1131, 1077, 960, 752, 694 cm⁻¹.

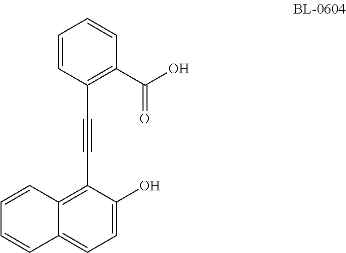 [SMILES: O=C(O)c1ccccc1C#Cc1c(O)ccc2ccccc12]

2-((2-hydroxynaphthalen-1-yl)ethynyl)benzoic acid. To a solution of methyl 2-((2-acetoxynaphthalen-1-yl)ethynyl)benzoate (100 mg, 0.29 mmol, 1.00 equiv, P=65%) in MeOH (1.6 mL) was added an aqueous solution of NaOH (10% wt, 0.8 mL). After 45 minutes, HCl 3N was added until pH=1. The reaction was extracted with EtOAc (×3). The combined organic layers were washed with brine and dried over Na₂SO₄, filtered and concentrated. The residue was purified by silica gel column chromatography (5/5 EtOAc/DCM) to give the desired product as a yellow solid in 37% yield (20 mg). ¹H NMR (600 MHz, DMSO) δ 13.44 (s, 1H), 10.09 (s, 1H), 8.46 (d, J=8.3 Hz, 1H), 7.99 (d, J=7.8 Hz, 1H), 7.88-7.83 (m, 2H), 7.80 (d, J=7.6 Hz, 1H), 7.65 (t, J=7.4 Hz, 1H), 7.54 (t, J=7.5 Hz, 1H), 7.51 (t, J=7.6 Hz, 1H), 7.38 (t, J=7.4 Hz, 1H), 7.24 (d, J=8.9 Hz, 1H) ppm. ¹³C NMR (151 MHz, DMSO) δ 167.53, 158.22, 134.18, 133.76, 132.10, 131.65, 130.90, 130.44, 128.18, 128.15, 127.56, 127.41, 124.87, 123.68, 123.42, 117.83, 102.44, 97.67, 89.53 ppm. HRMS (ES−) calculated for C₁₉H₁₁O₃ [M−H]⁻ 287.0714, found 287.0713. IR (neat) ν 3387, 1696, 1488, 1261, 1208, 817, 748, 611 cm⁻¹.

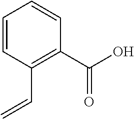

2-vinylbenzoic acid. This procedure has been adapted from the following article: J. Comb. Chem., 2007, 9 (06), pp 1060-1072. To a suspension of t-BuOK (3.59 g, 31.96 mmol, 2.40 equiv) in anhydrous THE (15.6 mL) was added a suspension of Methyltriphenylphosphonium bromide (7.61 g, 21.31 mmol, 1.60 equiv) in anhydrous THF (30.4 mL) at room temperature. The mixture was stirred 90 minutes. After that, a solution of 2-formylbenzoic acid (2.00 g, 13.32 mmol, 1.00 equiv) in anhydrous THE (7 mL) was slowly added. The reaction was heated at 60° c. for 20 h. The reaction was cooled to room temperature, quenched with acetic acid (0.8 mL) and filtered through celite. The filtrate was concentrated. The crude was solubilized with EtOAC and washed with saturated solution of NaHCO3 (×3). The aqueous layer was acidified with 1.0 M HCl solution and extracted with EtOAc (×3). The combined organic layers were washed with brine, dried over MgSO4, filtered and concentrated. The residue was purified by silica gel column chromatography (317 EtOAc/Hexanes) to give the desired product as a white solid in 12% yield (227 mg). ¹H NMR (599 MHz, CDCl₃) δ 8.06 (d, J=8.0 Hz, 1H), 7.64-7.52 (m, 3H), 7.37 (t, J=7.5 Hz, 1H), 5.67 (d, J=17.3 Hz, 1H), 5.39 (d, J=11.2 Hz, 1H) ppm. HRMS (ES−) calculated for C₉H₇O₂ [M−H]⁻ 147.0452, found 147.0452. IR (neat) ν 2989, 1685, 1566, 1485, 1404, 1305, 1269, 906, 767, 711 cm⁻¹.

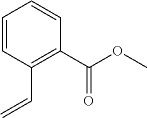

methyl 2-vinylbenzoate. To a suspension of 2-vinylbenzoic acid (200 mg, 1.35 mmol, 1.0) equiv), Cs₂CO₃ (1.76 g, 5.40 mmol, 4.00 equiv) in anhydrous DMF (2.00 mL) at room temperature was added MeI (336 μL, 5.40 mmol, 4.00 equiv). The reaction was stirred at room temperature overnight. The reaction was quenched with a HCl solution (1N) and extraction with EtOAc (3×). The combined organic layers were washed with a saturated solution of NaHCO₃, water, brine, dried over Na₂SO₄, filtered and concentrated to give without further purification the desired product as a yellow liquid in 81% yield (177 mg). ¹H NMR (600 MHz, CDCl₃) δ 7.88 (dd, J=7.9, 0.8 Hz, 1H), 7.59 (d, J=7.8 Hz, 1H), 7.51-7.43 (m, 2H), 7.32 (t, J=7.6 Hz, 1H), 5.66 (dd, J=17.4, 1.0 Hz, 1H), 5.36 (dd, J=11.0, 1.1 Hz, 1H), 3.90 (s, 3H) ppm. ¹³C NMR (151 MHz, CDCl₃) δ 167.99, 139.68, 135.97, 132.26, 130.43, 128.66, 127.53, 127.34, 116.63, 52.25 ppm. HRMS (ES+) calculated for C₁₀H₁₁O₂ [M+H]⁺ 163.0754, found 163.0756. IR (neat) ν 2951, 1716, 1482, 1433, 1250, 1130, 1076, 916, 768, 713, 665 cm⁻¹.

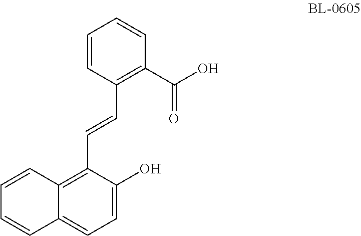 [SMILES: O=C(O)c1ccccc1/C=C/c1c(O)ccc2ccccc12]

(E)-2-(2-(2-hydroxynaphthalen-1-yl)vinyl)benzoic acid. To a solution of methyl (E)-2-(2-(2-acetoxynaphthalen-1-yl)vinyl)benzoate (65 mg, 0.18 mmol, 1.00 equiv) in MeOH (0.95 mL) was added a solution of NaOH (10% wt, 0.50 mL). The reaction was stirred at room temperature for 1 h. After that, MeOH (0.2 mL) and a solution of NaOH (10% wt, 0.50 mL) were added and the reaction was heated at 50° C. for 30 minutes. HCl solution (3N) was added until pH=1 and the reaction was extracted with EtOAc (×3). The combined organic layers were washed with brine, dried over Na2SO4, filtered and concentrated. The crude product was purified by silica gel column chromatography (0-40% of EtOAc in DCM) to give the desired product as a beige solid in 66% yield (35 mg). ¹H NMR (600 MHz, DMSO) δ 13.05 (s, 1H), 10.02 (s, 1H), 8.29 (d, J=8.6 Hz, 1H), 7.96 (d, J=2.7 Hz, 1H), 7.94 (d, J=5.6 Hz, 1H), 7.85-7.82 (m, 1H), 7.81 (d, J=8.0 Hz, 1H), 7.73 (d, J=8.8 Hz, 1H), 7.61 (t, J=7.4 Hz, 1H), 7.50 (d, J=16.5 Hz, 1H), 7.45 (t, J=7.4 Hz, 1H), 7.39 (t, J=7.6 Hz, 1H), 7.31 (t, J=7.4 Hz, 1H), 7.23 (d, J=8.9 Hz, 1H) ppm. ¹³C NMR (151 MHz, DMSO) δ 168.93, 153.32, 138.89, 132.42, 131.93, 131.91, 130.10, 129.71, 129.01, 128.45, 128.29, 127.13, 126.62, 126.57, 124.55, 123.49, 122.73, 118.23, 116.29 ppm. HRMS (ES−) calculated for C₁₉H₁₃O₃ [M−H]⁻ 289.0870, found 289.0871. IR (neat) ν 1672, 1250, 1201, 1137, 814, 739 cm⁻¹.

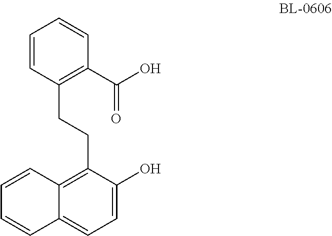 [SMILES: O=C(O)c1ccccc1CCc1c(O)ccc2ccccc12]

2-(2-(2-hydroxynaphthalen-1-yl)ethyl)benzoic acid. To a suspension of 2-((2-hydroxynaphthalen-1-yl)ethynyl)benzoic acid (35 mg, 0.12 mmol, 1.00 equiv) in MeOH (1.5 mL, previously degassed) was added Pd/C (10% wt, 13 mg, 0.01 mmol, 0.10 equiv). The reaction was stirred at room temperature under H₂ (atm pressure) overnight. After that the reaction was filtered through celite and the filtrate was concentrated. The crude product was purified by silica gel column chromatography (0-30% of EtOAc in DCM) to give the desired product as a white solid in 42% yield (15 mg). ¹H NMR (600 MHz, DMSO) δ 13.08 (s, 1H), 9.58 (s, 1H), 8.15 (d, J=8.6 Hz, 1H), 7.83 (dd, J=7.5, 0.8 Hz, 1H), 7.77 (d, J=8.0 Hz, 1H), 7.64 (d, J=8.8 Hz, 1H), 7.49 (td, J=7.5, 0.9 Hz, 1H), 7.44-7.41 (m, 1H), 7.35 (d, J=7.5 Hz, 1H), 7.32 (t, J=7.5 Hz, 1H), 7.26 (t, J=7.4 Hz, 1H), 7.18 (d, J=8.8 Hz, 1H), 3.25-320 (m, 2H), 3.14-3.09 (m, 2H) ppm. ¹³C NMR (151 MHz, DMSO) δ 169.20, 15228, 143.22, 133.27, 131.80, 130.93, 130.59, 130.13, 128.27, 128.13, 127.28, 126.07, 126.02, 122.82, 122.17, 119.18, 118.04, 34.07, 26.99 ppm. HRMS (ES−) calculated for C₁₉H₁₅O₃ [M−H]⁻ 291.1027, found 291.1025. IR (neat) ν 1678, 1596, 1388, 1273, 1250, 1199, 1144, 812, 739, 710 cm⁻¹.

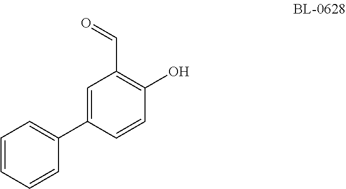 [SMILES: O=Cc1cc(-c2ccccc2)ccc1O]

4-hydroxy-[1,1′-biphenyl]-3-carbaldehyde. This procedure has been adapted from the following article. Tetrahedron Letters 42 (2001) 2093-209. A mixture of 5-bromo-2-hydroxybenzaldehyde (100 mg, 0.50 mmol, 1.00 equiv), phenylboronic acid (61 mg, 0.50 mmol, 1.00 equiv), Na₂CO₃ (79 mg, 0.75 mmol, 1.50 equiv) and Pd(dppf)Cl₂.DCM (2 mg, 0.02 mmol, 0.05 equiv) in mixture of DME/H₂O (3/1, 1.00 mL, previously degassed) were stirring at 100° C. for 4 hours. The reaction was cooled to room temperature. Water was added and the mixture was extracted with DCM (3×). The combined organic layers were washed with brine, dried over Na₂SO₄, filtered and concentrated. The crude product was purified by silica gel column chromatography (3/7 DCM/Hexanes) to give the desired product as a pale yellow solid in 27% yield (27 mg). ¹H NMR (600 MHz, CDCl₃) δ 9.97 (s, 1H), 7.79-7.74 (m, 2H), 7.56 (d, J=7.5 Hz, 2H), 7.46 (t, J=7.6 Hz, 2H), 7.37 (t, J=7.3 Hz, 1H), 7.08 (d, J=8.5 Hz, 1H) ppm. ¹³C NMR (151 MHz, CDCl₃) δ 196.81, 161.06, 139.41, 135.85, 133.39, 131.98, 129.10, 127.51, 126.70 ppm. HRMS (ES−) calculated for C₁₃H₉O₂ [M−H]⁻ 197.0608, found 197.0609. IR (neat) ν 3100, 1679, 1650, 1588, 1472, 1375, 1259, 1176, 904, 766, 752, 693, 674, 585 cm⁻¹.

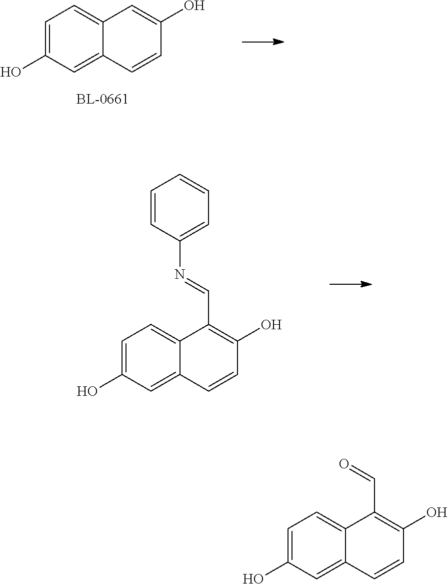 [SMILES: Oc1ccc2cc(O)ccc2c1 ; Oc1ccc2c(/C=N/c3ccccc3)c(O)ccc2c1 ; O=Cc1c(O)ccc2cc(O)ccc12]

1-((phenylimino)methyl)naphthalene-2,6-diol. Naphthalene-2,6-diol (1.00 g, 6.24 mmol) and N,N′-Diphenylformamidine (1.74 g, 8.74 mmol) was stirred at 130° C. After 5 hours, the reaction mixture was cooled to room temperature, followed by addition of of 10 mL acetone. The resulting orange-red precipitate product was used without further purification. To 1-((phenylimino)methyl)naphthalene-2,6-diol (0.500 g, 1.90 mmol) in ether (6.5 mL) was added 0.34 mL concentrated sulfuric acid and 0.34 mL water. The resulting mixture was allowed to stir at r.t. for 24 hours. The ether layer was separated, followed by evaporation under vacuum. Resulting solid was purified via reversed phased HPLC to yield 2,6-dihydroxy-1-naphthaldehyde as a yellow-brown solid (0.103 g, 29%). ¹H NMR (600 MHz, Methanol-d4) δ10.77 (s, 1H), 8.42 (d, J=9.3 Hz, 1H), 7.84 (d, J=9.0 Hz, 1H), 7.17 (dd, J=9.0, 2.7 Hz, 1H), 7.10 (t, J=2.7 Hz, 1H), 7.03 (d, J=9.0 Hz, 1H) ppm. ¹³C NMR (150 MHz, Methanol-d4) δ 195.45, 163.68, 155.41, 138.76, 130.99, 128.04, 122.42, 121.65, 119.98, 113.17, 112.17 ppm. HRMS (ES⁻) calculated for [C₁₁H₇O₃]⁺ 187.0401, found 187.0402.

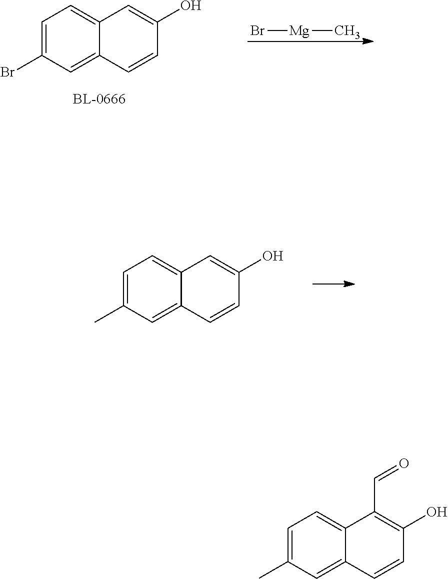

2-hydroxy-6-methyl-1-naphthaldehyde. To 6-bromonaphthalen-2-ol (1.00 g, 4.48 mmol) and Pd(dppf)Cl₂.CH₂Cl₂ (0.366 g, 0.448 mmol) in anh. THF (30 mL) was added methylmagnesium bromide (1 mL, 1M) at 0° C. The reaction was refluxed for 5 hours. The mixture was quenched with sat. NH4Cl and extracted with ethyl acetate. Purification via column chromatography yielded intermediate 6-methylnaphthalen-2-ol (0.220 g, 31%).

General Procedure B: To sodium hydroxide (0.493 g, 12.3 mmol) in 1 mL water was added 6-methylnaphthalen-2-ol (0.150 g, 0.948 mmol) in 0.5 mL ethanol. The resulting mixture was stirred at 80° C. Chloroform (0.120 mL) was added dropwise. After stirring at 80° C. for 1 hour, the mixture was cooled to r.t. Mixture was acidified with 1M HCl and extracted with ethyl acetate. Purification via column chromatography yielded 2-hydroxy-6-methyl-1-naphthaldehyde as a yellow solid (0.100 g, 58%). ¹H NMR (600 MHz, Chloroform-d) δ 10.80 (s, 1H), 8.25 (d, J=8.6 Hz, 1H), 7.91 (d, J=9.0 Hz, 1H), 7.58 (s, 1H), 7.46 (dd, J=8.6, 2.1 Hz, 1H), 7.11 (d, J=9.0 Hz, 1H), 2.50 (s, 3H). ¹³C NMR (151 MHz, CDCl₃) δ 193.51, 164.46, 134.27, 131.30, 130.99, 128.82, 128.21, 118.63, 113.01, 111.45, 21.29. HRMS (ES+) calculated for [C₁₂H₁₁O₂]⁻ 187.0754, found 187.0756.

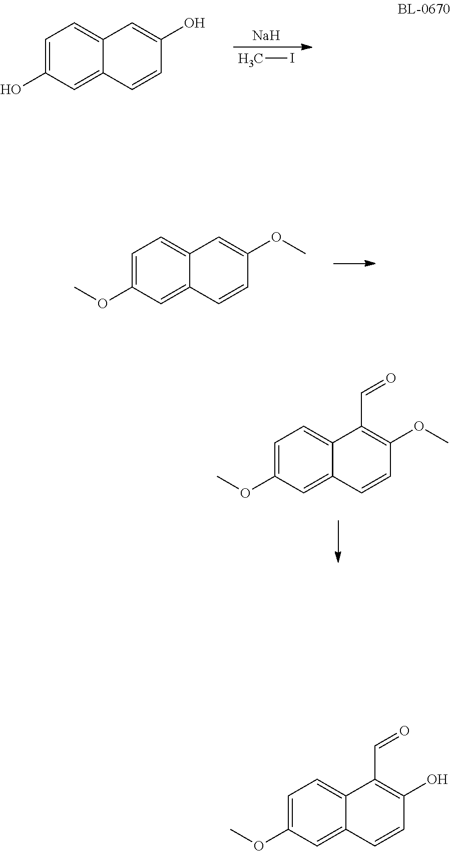

2-hydroxy-6-methoxy-1-naphthaldehyde. To dihydroxy-2,6-naphthalene (1.00 g. 6.24 mmol) in anh. DMF was added sodium hydride (0.62 g, 15.6 mmol) in three portions at 0° C. The reaction mixture was warmed to r.t, and stirred for 30 min. The flask was cooled to 0° C., to which iodomethane (0.980 mL, 15.6 mmol) was added dropwise. The mixture was stirred at r.t for 16 hours. 0.5 mL of methanol was added. The resulting mixture was washed with water and extracted with ethyl acetate. Purification via column chromatography yielded intermediate dimethoxy-2,6-naphthalene (0.86 g, 73%). Dimethoxy-2,6-naphthalene (0.500 g, 2.66 mmol), phosphoryl trichloride (0.273 mL, 2.92 mmol), and N-methylformanilide (0.361 mL, 2.92 mmol) were stirred at 100° C. for 16 hours. Reaction mixture was cooled to r.t. and 5 mL DMF was added. Mixture was poured into cold 1M HCl and stirred vigorously, followed by extracted with ethyl acetate. Crude was purified via column chromatography to yield 2,6-dimethoxy-1-naphthaldehyde (0.470 g, 82%). 2,6-dimethoxy-1-naphthaldehyde (0.200 g, 0.925 mmol), magnesium bromide (0.341 g, 1.85 mmol), and sodium iodide (0.277 g, 1.85 mmol) was dissolved in anh. Acetonitrile (6 mL). Mixture was stirred at 100° C. for two hours. Water was added, followed by extraction with ethyl acetate. Purification by column chromatography yielded desired product 2-hydroxy-6-methoxy-1-naphthaldehyde as a yellow solid (0.160 mg, 86%). ¹H NMR (600 MHz, Chloroform-d) δ 12.90 (s, 1H), 10.78 (s, 1H), 8.27 (d, J=9.2 Hz, 1H), 7.90 (d, J=9.2 Hz, 1H), 7.29 (dd, J=9.0, 2.8 Hz, 1H), 7.17-7.11 (m, 2H), 3.92 (s, 3H) ppm. ¹³C NMR (150 MHz, CDCl₃) δ 193.35, 163.16, 156.55, 138.03, 129.02, 127.64, 120.87, 120.17, 119.60, 111.59, 108.29, 55.43 ppm. HRMS (ES−) calculated for [C₁₂H₉O₃]−201.0557, found 201.0556.

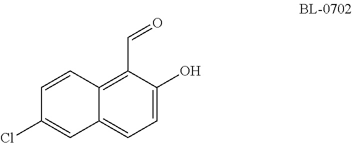 [SMILES: O=Cc1c(O)ccc2cc(Cl)ccc12]

6-chloro-2-hydroxy-1-naphthaldehyde. Synthesis closely followed general procedure B from 6-chloro-2-naphthol to yield a yellow solid (85 mg, 37%). ¹H NMR (600 MHz, Chloroform-d) δ 13.10 (s, 1H), 10.75 (s, 1H), 8.26 (d, J=9.2 Hz, 1H), 7.88 (d, J=9.2 Hz, 1H), 7.77 (s, 1H), 7.55 (d, J=8.9 Hz, 11H), 7.17 (d, J=8.9 Hz, 11H) ppm. ¹³C NMR (151 MHz, CDCl₃) δ 192.98, 164.80, 137.95, 131.09, 130.20, 129.61, 128.57, 120.54, 120.24, 111.20 ppm.

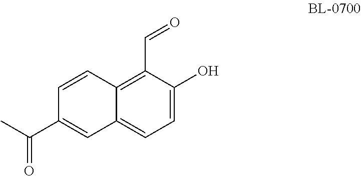 [SMILES: CC(=O)c1ccc2c(C=O)c(O)ccc2c1]

6-acetyl-2-hydroxy-1-naphthaldehyde. To 1-(6-methoxynaphthalen-2-yl)ethan-1-one (0.200 g, 0.99 mmol) in anh. DCM (10 mL) was added boron tribromide at −78° C. Mixture was stirred at −78° C. for 10 min, then at r.t. for 2 hr. Water was added, followed by extraction with DCM. Purification by column chromatography to yield intermediate 1-(6-hydroxynaphthalen-2-yl)ethan-1-one (0.155 g, 83%). Formation of final product closely followed general procedure B. Purification by column chromatography yielded desired product 6-acetyl-2-hydroxy-1-naphthalidehyde (0.047 g, 29%). 1H NMR (600 MHz, Chloroform-d) δ 13.32 (s, ¹H), 10.83 (s, 1H), 8.44-8.40 (m, 2H), 8.19 (d, J=9.5 Hz, 1H), 8.10 (d, J=9.2 Hz, 1H), 7.23 (d, J=9.2 Hz, 1H), 2.72 (s, 3H) ppm. ¹³C NMR (150 MHz, CDCl₃) δ 197.33, 193.35, 166.66, 140.27, 135.85, 133.31, 131.17, 127.68, 127.02, 120.47, 119.17, 111.44, 26.67 ppm. HRMS (ES⁻) calculated for [C₁₃H₉O₃]⁻ 213.0557, found 213.0556.

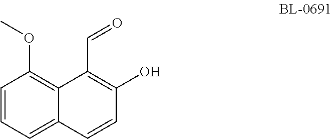

2-hydroxy-8-methoxy-1-naphthaldehyde. Same procedure as in the synthesis of 2-hydroxy-6-methoxy-1-naphthaldehyde. ¹H NMR (600 MHz, Chloroform-d) δ 11.23 (s, 1H), 7.89 (d, J=9.2 Hz, 1H), 7.39 (d, J=7.9 Hz, 1H), 7.33 (d, J=7.9 Hz, 1H), 7.11 (d, J=8.9 Hz, 1H), 7.06 (d, J=7.9 Hz, 1H), 4.00 (s, 3H) ppm. ¹³C NMR (150 MHz, CDCl₃) δ 199.69, 166.14, 155.88, 138.80, 129.90, 124.26, 123.28, 122.51, 120.09, 113.58, 109.46, 55.73 ppm. HRMS (ES⁺) calculated for [C₁₂H₁₁O₃]⁺ 203.0703, found 203.0704.

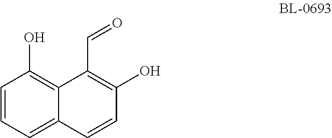 [SMILES: O=Cc1c(O)ccc2cccc(O)c12]

2,8-dihydroxy-1-naphthaldehyde. To 2-hydroxy-8-methoxy-1-naphthaldehyde (0.039 g, 0.19 mmol) in anh. DCM (1.8 mL) was added boron tribromide (0.092 mL, 0.96 mmol) at −78° C. After 10 min at −78° C., the reaction mixture was warmed and stirred at r.t. for 16 hours. Cold water was added, followed by extraction with ethyl acetate. Crude was purified via column chromatography to yield 2,8-dihydroxy-1-naphthaldehyde as a yellow solid (0.029 g, 80%). ¹H NMR (600 MHz, DMSO-d6) δ 13.90 (s, 1H), 11.26 (s, 1H), 10.78 (s, 1H), 8.06 (d, J=9.1 Hz, 1H), 7.37 (d, J=8.0 Hz, 1H), 7.23 (t, J=7.8 Hz, 1H), 7.09 (dd, J=12.4, 8.4 Hz, 2H) ppm. ¹³C NMR (150 MHz, DMSO) δ 199.39, 164.88, 153.34, 139.59, 129.92, 124.76, 121.38, 120.84, 119.20, 114.09, 113.30 ppm.

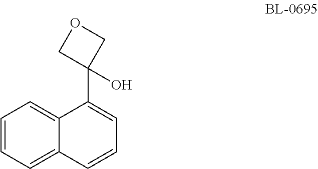 [SMILES: OC1(c2cccc3ccccc23)COC1]

3-(naphthalen-1-yl)oxetan-3-ol. To 1-bromonaphthalene (0.500 g, 2.41 mmol) in anh. ether (13 mL) was added butyllithium (1.52 mL, 1.75M) at 0° C. The resulting mixture was stirred at 0° C. for 10 min, to which oxetan-3-one (0.209 mg, 2.90 mmol) was added slowly. The reaction mixture was warmed to r.t. and stirred for 1 hr. Water was added, followed by extraction with ether. Purification via column chromatography yielded desired product 3-(naphthalen-1-yl)oxetan-3-ol as a white solid (0.478 g, 99%). ¹H NMR (600 MHz, Chloroform-d) δ7.92 (d, J=7.3 Hz, 1H), 7.85 (d, J=8.3 Hz, 1H), 7.67 (d, J=9.2 Hz, 1H), 7.54 (h, J=6.1, 5.6 Hz, 2H), 7.44 (t, J=7.7 Hz, 1H), 7.32 (d, J=7.3 Hz, 1H), 5.15 (d, J=7.2 Hz, 2H), 4.99 (d, J=7.2 Hz, 2H), 3.90 (d, J=3.1 Hz, 1H) ppm. ¹³C NMR (150 MHz, CDCl₃) δ 137.14, 134.39, 130.11, 129.34, 129.11, 126.58, 126.04, 124.89, 124.64, 123.76, 83.89, 76.89 ppm. HRMS (ES⁻) calculated for [C₁₃H₁₁O₂]⁻ 199.0765, found 199.0767.

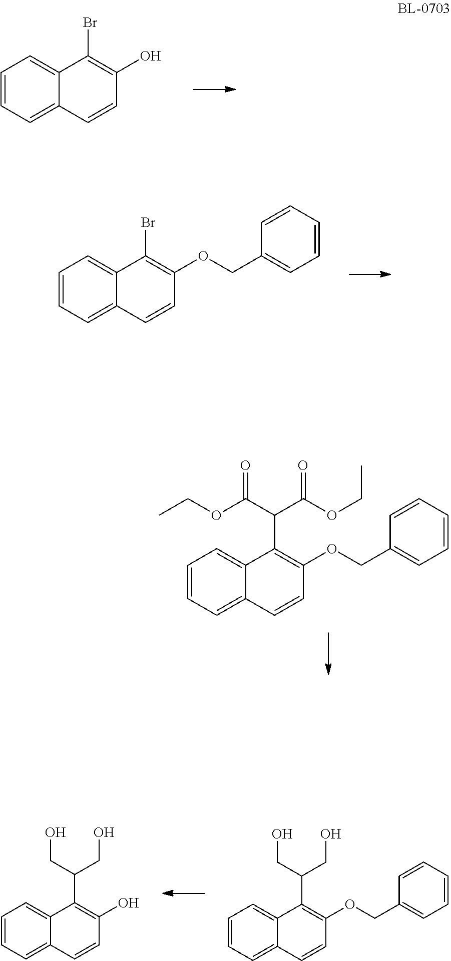 [SMILES: Oc1ccc2ccccc2c1Br ; Brc1c(OCc2ccccc2)ccc2ccccc12 ; CCOC(=O)C(C(=O)OCC)c1c(OCc2ccccc2)ccc2ccccc12 ; OCC(CO)c1c(O)ccc2ccccc12 ; OCC(CO)c1c(OCc2ccccc2)ccc2ccccc12]

2-(2-hydroxynaphthalen-1-yl)propane-1,3-diol. To 1-bromonaphthalen-2-ol (2.00 g, 8.97 mmol) and potassium carbonate (2.48 g, 17.9 mmol) in anhydrous DMF (20 mL) was added benzyl bromide (1.17 mL, 9.86 mmol). The reaction mixture was allowed to stir at 70° C. overnight. Water was added to the resulting mixture, followed by extraction with ether. Solvent was removed under vacuum, and the resulting light brown solid 2-(benzyloxy)-1-bromonaphthalene was used in the next reaction without further purification. To sodium hydride (60% weight in oil, 766 mg, 19.2 mmol) in degassed, anhydrous dioxane (18 mL) was added diethyl malonate (2.91 mL, 19.2 mmol) dropwise at 60° C. Copper (II) bromide (1.10 g, 7.66 mmol) was added in one portion, followed by the addition of 2-(benzyloxy)-1-bromonaphthalene in 10 mL dioxane dropwise at 60° C. The reaction mixture was stirred at 100° C. overnight. The reaction was cooled to room temperature, to which 1 mL of concentrated HCl was added slowly. The resulting mixture was quickly filtered through celite, followed by extraction with ethyl acetate. The crude was purified via column chromatography to yield diethyl 2-(2-(benzyloxy)naphthalen-1-yl)malonate as a off-white solid (1.19 g, 48%). LAH (4M solution in ether, 1.5 mL, 6.06 mmol) was added dropwise to diethyl 2-(2-(benzyloxy)naphthalen-1-yl)malonate (1.19 g, 3.03 mmol) in anhydrous ether (10 mL) at −30° C. The reaction mixture was warmed to r.t. and allowed to stir overnight. Resulting mixture was diluted with ether, followed by the addition of water (10 mL) and 1M NaOH (10 mL). Product was extracted with ethyl acetate and purified via column chromatography was a colorless oil (0.416 g, 45%). To 2-(2-(benzyloxy)naphthalen-1-yl)propane-1,3-diol (0.120 g, 0.389 mmol) in ethanol (2 mL) was added Pd/C (10% weight. 25 mg, 0.023 mmol) in one portion. The reaction mixture was degassed with H₂ and stirred under H₂ atmosphere for 24 hours. Resulting mixture was quickly filtered through celite, washed with ethyl acetate, and concentrated under vacuum. Desired product 2-(2-hydroxynaphthalen-1-yl)propane-1,3-diol was purified via column chromatography to obtain a white solid (69 mg, 81%). ¹H NMR (600 MHz, Methanol-d4) δ 8.07 (d, J=7.9 Hz, 1H), 7.70 (d, J=8.3 Hz, 1H), 7.59 (d, J=9.0 Hz, 1H), 7.40 (t, J=7.8 Hz, 1H), 7.22 (t, J=7.5 Hz, 1H), 7.03 (d, J=9.0 Hz, 1H), 4.16 (t, J=8.8 Hz, 2H), 4.02 (dd, J=11.0, 5.7 Hz, 2H), 3.88 (s, 1H) ppm. ¹³C NMR (150 MHz, DMSO) δ 153.84, 134.69, 128.87, 128.80, 128.28, 126.36, 123.22, 122.49, 119.61, 119.27, 61.65 ppm. HRMS (ES⁻) calculated for [C₁₃H₁₃O₃]⁻ 217.0870, found 217.0870.

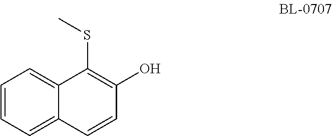

1-(methylthio)naphthalen-2-ol. To 2-naphthol (0.300 g, 2.08 mmol), sodium methane sulfinate (0.531 g, 520 mmol), and iodine (0.528 g, 2.08 mmol) was added formic acid (1.0 mL) and water (5 mL). The reaction mixture was stirred at 110° C. for 24 hr. The resulting mixture was cooled to r.t. and extracted with ethyl acetate. Crude was purified via column chromatography to yield a clear liquid (0.364 g, 92%). ¹H NMR (600 MHz, Chloroform-d) δ 8.34 (d, J=8.6 Hz, 1H), 7.79 (d, J=8.5 Hz, 2H), 7.58 (t, J=7.7 Hz, 1H), 7.42-7.35 (m, 2H), 7.27 (s, 1H), 2.29 (s, 3H) ppm.

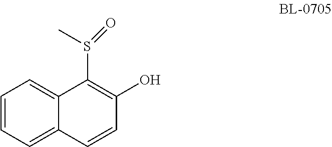

1-(methylsulfinyl)naphthalen-2-ol. 3-chlorobenzoperoxoic acid (0.139 g, 0.578 mmol) in anh. DCM (2.0 mL) was added slowly to 1-(methylthio)naphthalen-2-ol in anh. DCM (3.0 mL) at 0° C. The reaction was slowly allowed to warm to r.t. and was stirred overnight. The resulting mixture was diluted with DCM, washed with sodium bicarbonate, and extracted with DCM. Crude was purified via column chromatography to yield an off white solid (95 mg, 88%). ¹H NMR (600 MHz, Chloroform-d) δ 11.56 (s, 1H), 7.84 (d, J=8.7 Hz, 1H), 7.79 (d, J=8.2 Hz, 1H), 7.64 (d, J=8.7 Hz, 1H), 7.50 (t, J=7.6 Hz, 1H), 7.38 (t, J=7.6 Hz, 1H), 7.11 (d, J=9.2 Hz, 1H), 3.06 (s, 3H) ppm. ¹³C NMR (151 MHz, CDCl3) δ 160.56, 133.75, 129.94, 129.11, 128.14, 128.10, 124.16, 121.41, 119.82, 112.85, 76.95, 40.18 ppm. HRMS (ES⁻) calculated for [C₁₁H₉O₂S]⁻ 205.0329, found 205.0329.

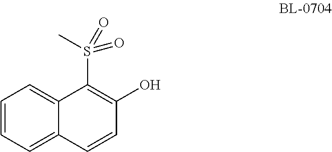

1-(methylsulfonyl)naphthalen-2-ol. To 1-(methylthio)naphthalen-2-ol (0.100 g, 0.526 mmol) in acetone (2.5 mL) was added Oxone (0.404 g, 1.31 mmol) in water (2.5 mL) at 0° C. The reaction was stirred at 0° C. for 20 min, then warmed and stirred at r.t. overnight. Resulting mixture was treated with 1M sodium sulfite and extracted with ethyl acetate. Crude was purified via column chromatography as an off white solid (95 mg, 81%). ¹H NMR (600 MHz, Chloroform-d) δ 10.79 (s, 1H), 8.51 (d, J=8.8 Hz, 1H), 7.96 (d, J=9.2 Hz, 1H), 7.81 (d, J=8.3 Hz, 1H), 7.65 (s, 1H), 7.45 (s, 1H), 7.15 (d, J=9.2 Hz, 1H), 3.31 (s, 3H) ppm. ¹³C NMR (151 MHz, CDCl₃) δ 158.21, 137.57, 129.89, 129.57, 129.43, 128.87, 124.67, 122.59, 120.35, 112.08, 45.00 ppm. HRMS (ES⁻) calculated for [C₁₁H₉O₃S]⁻ 221.0278, found 221.0279.

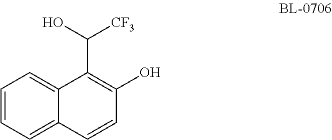 [SMILES: Oc1ccc2ccccc2c1C(O)C(F)(F)F]

1-(2,2,2-trifluoro-1-hydroxyethyl)naphthalen-2-ol. To 2-naphthol (0.500 g. 3.47 mmol) and 50 mg 4 A molecular sieves in anh. DCM (17 mL) was added titanium(IV) chloride (0.380 mL, 3.47 mmol) dropwise at r.t. The reaction was allowed to stir at r.t. for 30 mins, followed by the addition of Trifluoroacetaldehyde ethyl hemiacetal (0.45 mL, 3.47 mmol). The mixture was allowed to stir at r.t. for 3 hr. Water was added and product was extracted with DCM. Crude was purified via column chromatography as an off white solid (0.770 g, 92%). ¹H NMR (600 MHz, DMSO-d6) δ 10.12 (s, 1H), 8.41 (s, 1H), 7.71 (dd, J=13.1, 8.2 Hz, 2H), 7.34 (ddd, J=8.6, 6.7, 1.4 Hz, 1H), 7.21 (t, J=7.4 Hz, 1H), 7.12 (d, J=9.0 Hz, 1H), 6.88 (s, 1H), 5.98 (q, J=9.9 Hz, 1H) ppm. ¹³C NMR (150 MHz, DMSO) δ 132.89, 130.89, 129.09, 128.56, 128.24, 127.20, 125.93, 125.32, 123.44, 122.65, 117.67, 65.61 ppm. HRMS (ES⁻) calculated for [C₁₂H₈F₃O₂]⁻ 241.0482, found 241.0480.

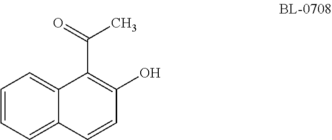

2,2,2-trifluoro-1-(2-hydroxynaphthalen-1-yl)ethan-1-one. To 2-hydroxy-1-naphthaldehyde (1.00 g. 5.81 mmol) in anh. DMF (30 mL) was added potassium carbonate (1.61 g, 11.6 mmol). The mixture was stirred at r.t. for 15 mins, followed by the addition of iodomethane (0.723 mL, 11.6 mmol). The reaction was stirred at 90° C. for 4 hr. Water was added, followed by extraction with ethyl acetate. Crude was used in the next step without further purification. To 2-methoxy-1-naphthaldehyde (0.500 g, 2.69 mmol) in anh. THF (5.0 mL) was added trimethyl(trifluoromethyl)silane (0.437 mL, 2.95 mmol) at 0° C. The reaction mixture was stirred at 0° C. for 15 mins, followed by the addition of TBAF (1.0 M in THF, 0.027 mL) at 0° C. The reaction was stirred at r.t. overnight. 3 mL of water was added, followed by 0.28 mL of TBAF at 0° C. The resulting mixture was stirred at r.t. for 4 hr. Product was extracted with ethyl acetate, and crude was used in the next step without further purification. To 2,2,2-trifluoro-1-(2-methoxynaphthalen-1-yl)ethan-1-ol (0.100 g. 0.390 mmol) in anh. DCM (3.0 mL) was added sodium carbonate (0.165 g, 1.56 mmol) and DNP (0.497 g, 1.17 mmol). The mixture was stirred at r.t. for 3 hr. Water was added, in which the mixture was allowed to stir at r.t. for another hr. Resulting mixture was extracted with DCM. Crude was purified via column chromatography as a white solid (44 mg, 44%). To 2,2,2-trifluoro-1-(2-methoxynaphthalen-1-yl)ethan-1-one (44 mg, 0.17 mmol) in anh. DCM (2.0 mL) was added boron tribromide (0.082 mL, 0.87 mmol) dropwise at −78° C. The mixture was warmed to r.t. overnight. Water was added at 0° C., followed by extraction with DCM. Crude was purified via column chromatography to yield a yellow solid (29 mg, 70%). ¹H NMR (599 MHz, Chloroform-d) δ 10.80 (s, 1H), 8.0) (d, J=9.0 Hz, 1H), 7.96 (d, J=8.8 Hz, 1H), 7.80 (d, J=8.2 Hz, 1H), 7.59 (ddd, J=8.5, 6.8, 1.5 Hz, 1H), 7.46 (t, J=7.5 Hz, 1H), 7.17 (d, J=9.0 Hz, 1H) ppm. ¹³C NMR (151 MHz, CDCl₃) δ 186.20, 185.96, 185.71, 185.47, 164.51, 139.74, 130.67, 129.29, 128.99, 128.42, 125.35, 124.61, 119.70, 119.06, 117.78, 115.86, 113.94, 111.46 ppm. HRMS (ES⁺) calculated for [C₁₂H₆F₃O₂]⁻ 239.0325, found 239.0326.

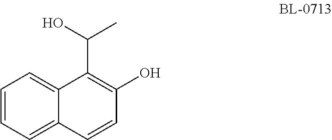

1-(1-hydroxyethyl)naphthalen-2-ol. To 1-(2-hydroxynaphthalen-1-yl)ethan-1-one (0.100 g, 0.537 mmol) in MeOH (3.0 mL) was added sodium borohydride (25 mg, 0.66 mmol) at 0° C. The reaction was stirred at 0° C. for 15 min, then stirred at r.t. for 1 hr. Water was added. Methanol was removed under reduced pressure. Crude was extracted with ethyl acetate and purified via column chromatography (56 mg, 55%). ¹H NMR (599 MHz, Chloroform-d) δ 9.19 (s, 1H), 7.78-7.75 (m, 1H), 7.69 (d, J=8.8 Hz, 2H), 7.45 (ddd, J=8.3, 6.7, 1.3 Hz, 11H), 7.32 (t, J=7.5 Hz, 1H), 7.12 (d, J=8.8 Hz, 1H), 5.97 (qd, J=6.7, 3.0 Hz, 1H), 2.69 (d, J=2.9 Hz, 1H), 1.69 (d, J=6.8 Hz, 3H) ppm. ¹³C NMR (151 MHz, CDCl₃) δ 154.01, 130.89, 129.63, 129.02, 128.76, 126.80, 123.02, 120.80, 119.98, 118.34, 69.14, 22.83 ppm. HRMS (ES⁺) calculated for [C₁₂H₁₁O₂]⁻ 187.0765, found 187.0765.

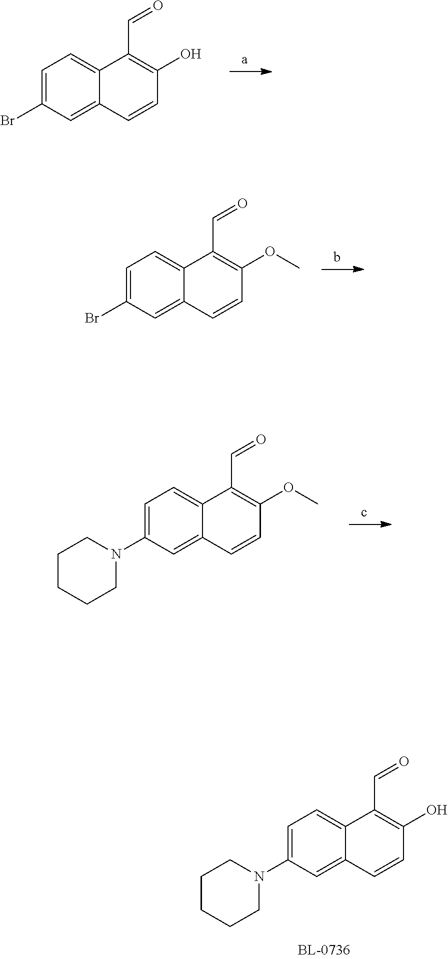 [SMILES: O=Cc1c(O)ccc2cc(Br)ccc12 ; COc1ccc2cc(Br)ccc2c1C=O ; COc1ccc2cc(N3CCCCC3)ccc2c1C=O ; O=Cc1c(O)ccc2cc(N3CCCCC3)ccc12]

Reagents and conditions: (a) 6-bromo-2-hydroxy-1-naphthaldehyde (1.00 eq), K₂CO₃ (2.00 eq), CH₃I (2.00), DMF, 90° C., 3 h. (b) 6-bromo-2-methoxy-1-naphthaldehyde (1.0) eq), Pd(OAc)₂ (0.02 eq), (R)-(+)-BINAP (0.03 eq), piperidine (4.00 eq), toluene, 100° C., 16 h. (c) of 2-methoxy-6-(piperidin-1-yl)-1-naphthaldehyde (1.00 eq), BBr₃ (5.00 eq), CH₂Cl₂, 0° C. to r.t., 16 h.

6-Bromo-2-methoxy-1-naphthaldehyde. To a solution of 6-bromo-2-hydroxy-1-naphthaldehyde (1.00 g, 3.98 mmol, 1.00 eq) in anh. DMF at r.t. under N₂, potassium carbonate (1.10 g, 7.97 mmol, 2.00 eq) and iodomethane (1.13 g, 7.97 mmol, 2.00 eq) were added. The reaction mixture was stirred at 90° C. for 3 hr. The resulting mixture was cooled to r.t., washed with water, and extracted with EtOAc (3×). The combined organic extracts were dried over Na₂SO₄, filtered, and concentrated in vacuo. Intermediate 6-bromo-2-methoxy-1-naphthaldehyde was used without further purification (quantitative yield). ¹H NMR (600 MHz, CDCl₃) δ 10.86 (s, 11H), 9.18 (d, J=9.2 Hz, 1H), 7.97 (d, J=8.9 Hz, 1H), 7.93 (d, J=2.6 Hz, 1H), 7.67 (dd, J=9.1, 2.5 Hz, 1H), 7.34 (d, J=9.1 Hz, 1H), 4.07 (s, 4H) ppm.

2-Methoxy-6-(piperidin-1-yl)-1-naphthaldehyde. To a solution of 6-bromo-2-methoxy-1-naphthaldehyde (0.050 g, 0.19 mmol, 1.00 eq) in anh. toluene at r.t. under N₂, cesium carbonate (0.220 g, 0.66 mmol, 3.50 eq), palladium(II) acetate (0.001 g, 0.0038 mmol, 0.02 eq), 2,2′-bis(diphenylphosphaneyl)-1,1′-binaphthalene (0.004 g, 0.0057 mmol, 0.03 eq), and piperidine (0.064 g, 0.75 mmol, 4.00 eq) were added. The reaction mixture was stirred at 100° C. overnight. The resulting mixture was warmed to r.t., diluted with water, and extracted with EtOAc (3×). The combined organic extracts were dried over Na₂SO₄, filtered, and concentrated in vacuo. Purification by silica gel column chromatography yielded intermediate 2-methoxy-6-(piperidin-1-yl)-1-naphthaldehyde as a yellow solid (0.013 g, 0.049 mmol, 26%). ¹H NMR (600 MHz, CDCl₃) δ 10.85 (s, 1H), 9.13 (d, J=9.4 Hz, 1H), 7.90 (d, J=9.1 Hz, 1H), 7.42 (dd, J=9.5, 2.8 Hz, 1H), 7.22 (d, J=9.4 Hz, 1H), 7.06 (d, J=2.6 Hz, 1H), 4.01 (s, 3H), 3.25-3.21 (m, 5H), 1.76 (p, J=5.7 Hz, 5H), 1.62 (td, J=6.2, 3.1 Hz, 3H) ppm.

2-Hydroxy-6-(piperidin-1-yl)-1-naphthaldehyde (BL-0736). To a solution of 2-methoxy-6-(piperidin-1-yl)-1-naphthaldehyde (0.013 g, 0.037 mmol, 1.00 eq) in anh. CH₂Cl₂ (0.7 mL) at 0° C. under N₂, boron tribromide (0.047 g, 0.19 mmol, 5.00 eq) was added dropwise. The reaction mixture was stirred at r.t. overnight. The resulting mixture was quenched with water and extracted with EtOAc (3×). The combined organic extracts were dried over Na₂SO₄, filtered, and concentrated in vacuo. Purification by silica gel column chromatography yielded the title compound as a yellow solid (0.003 g, 0.014 mmol, 32%). ¹H NMR (600 MHz, CDCl₃) δ 12.85 (s, 1H), 10.76 (s, 1H), 8.22 (d, J=9.2 Hz, 1H), 7.85 (d, J=9.2 Hz, 1H), 7.41 (dd, J=9.1, 2.8 Hz, 1H), 7.12 (d, J=3.0 Hz, 1H), 7.07 (d, J=8.9 Hz, 1H), 3.25-3.20 (m, 4H), 1.77 (p, J=5.5 Hz, 4H), 1.63 (td, J=7.1, 5.9, 4.3 Hz, 3H) ppm. ¹³C NMR (150 MHz, CDCl₃) δ 193.51, 163.11, 149.29, 138.43, 129.33, 126.88, 122.67, 119.54, 119.30, 112.77, 111.65, 51.06, 25.97, 24.38 ppm.

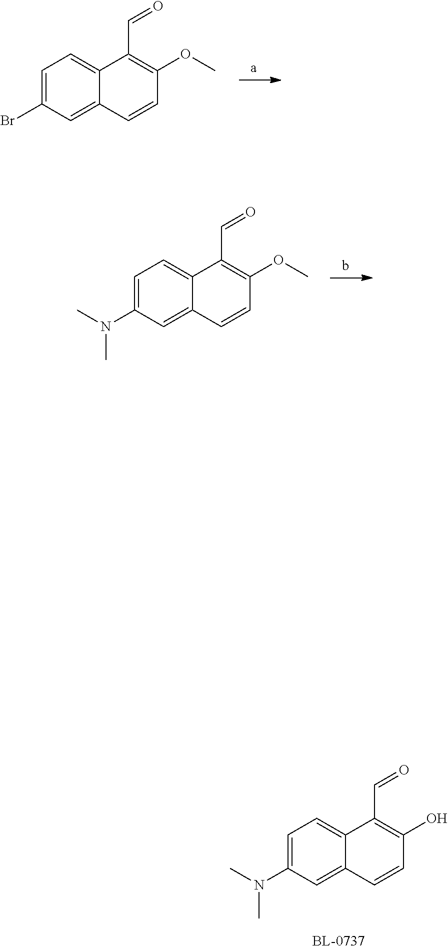

Reagents and conditions: (a) 6-bromo-2-methoxy-1-naphthaldehyde (1.00 eq), Pd(OAc)₂ (0.02 eq), (R)-(+)-BINAP (0.03 eq), dimethylamine (4.00 eq), toluene, 100° C., 16 h. (c) 6-(dimethylamino)-2-methoxy-1-naphthaldehyde (1.00 eq), BBr₃ (5.00 eq), CH₂Cl₂, 0° C. to r.t., 16 h.

6-(Dimethylamino)-2-methoxy-1-naphthaldehyde. To a solution of 6-bromo-2-methoxy-1-naphthaldehyde (0.100 g, 0.377 mmol, 1.00 eq) in anh. toluene (3.0 mL) at r.t. under N₂, cesium carbonate (0.430 g, 1.51 mmol, 3.50 eq), palladium(II) acetate (0.002 g, 0.008 mmol, 0.02 eq), 2,2′-bis(diphenylphosphaneyl)-1,1′-binaphthalene (0.007 g, 0.011 mmol, 0.03 eq), and dimethylamine (0.068 g, 1.51 mmol, 4.00 eq) were added. The reaction mixture was stirred at 100° C. overnight. The resulting mixture was warmed to r.t., diluted with water, and extracted with EtOAc (3×). The combined organic extracts were dried over Na₂SO₄, filtered, and concentrated in vacuo. Purification by silica gel column chromatography yielded intermediate 6-(dimethylamino)-2-methoxy-1-naphthaldehyde (0.023 g, 0.090 mmol, 27%). ¹H NMR (600 MHz, CDCl₃) δ 10.85 (s, 1H), 9.15 (d, J=9.6 Hz, 1H), 7.90 (d, J=9.2 Hz, 1H), 7.30 (dd, J=9.4, 2.8 Hz, 1H), 7.21 (d, J=9.1 Hz, 1H), 6.87 (d, J=3.0 Hz, 1H), 4.01 (s, 3H), 3.03 (s, 6H) ppm.

6-(Dimethylamino)-2-hydroxy-1-naphthaldehyde (BL-0737). To a solution of 6-(dimethylamino)-2-methoxy-1-naphthaldehyde (0.022 g, 0.096 mmol, 1.00 eq) in anh. CH₂Cl₂ (1.2 mL) at 0° C. under N₂ boron tribromide (0.120 g, 0.48 mmol, 5.00 eq) was added dropwise. The reaction mixture was stirred at r.t. overnight. The resulting mixture was quenched with water and extracted with EtOAc (3×). The combined organic extracts were dried over Na₂SO₄, filtered, and concentrated in vacuo. Purification by silica gel column chromatography yielded the title compound as a yellow solid (0.009 g, 0.042 mmol, 44%). ¹H NMR (600 MHz, CDCl₃) δ 12.81 (s, 1H), 10.76 (s, 1H), 8.22 (d, J=9.2 Hz, 1H), 7.84 (d, J=9.2 Hz, 1H), 7.27 (d, J=3.0 Hz, 1H), 7.06 (d, J=8.9 Hz, 1H), 6.93 (d, J=3.0 Hz, 1H), 3.04 (s. 6H) ppm. ¹³C NMR (150 MHz, CDCl₃) δ 193.47, 162.53, 147.86, 138.18, 129.62, 125.06, 119.66, 119.39, 118.76, 111.63, 109.01, 40.97 ppm. HRMS (ES+) calculated for [C₁₃H₁₄NO₂]+216.1019, found 216.1020. IR (neat) ν 2917.21, 2849.27, 1631.73, 1615.03, 1586.96, 1462.86, 1300.56, 1246.73, 1155.39, 1181.49, 806.59, 738.78, 679.94, 599.31, 540.29, 481.49 cm⁻¹.

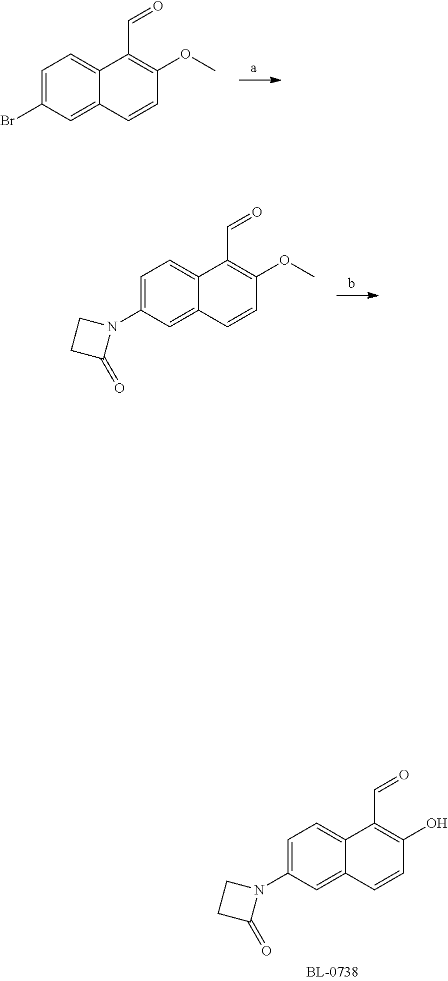 [SMILES: COc1ccc2cc(Br)ccc2c1C=O ; COc1ccc2cc(N3CCC3=O)ccc2c1C=O ; O=Cc1c(O)ccc2cc(N3CCC3=O)ccc12]

Reagents and conditions: (a) 6-bromo-2-methoxy-1-naphthaldehyde (1.00 eq), Pd₂(dba)₃ (0.02 eq), Xantphos (0.03 eq), azetidin-2-one (1.20 eq), 1,4-dioxane, 100° C., 48 h. (c) 2-methoxy-6-(2-oxoazetidin-1-yl)-1-naphthaldehyde (1.00 eq), BBr₃, (5.00 eq), CH₂CL, 0° C. to r.t., 16 h.

2-Methoxy-6-(2-oxoazetidin-1-yl)-1-naphthaldehyde. A solution of 6-bromo-2-methoxy-1-naphthaldehyde (0.100 g, 0.377 mmol, 1.00 eq), Pd₂(dba)₃ (0.007 g, 0.008 mmol, 0.02 eq), Xantphos (0.007 g, 0.011 mmol, 0.03 eq), azetidin-2-one (0.032 g, 0.453 mmol, 1.20 eq), and Cs₂CO₃ (0.430 g, 1.32 mmol, 3.50 eq) in 1,4-dioxane (2.0 mL) was stirred at 100° C. for 48 h. The reaction mixture was cooled to r.t. and stirred for an additional 24 h. The resulting mixture was filtered through celite, diluted with water, and extracted with EtOAc (3×). The combined organic extracts were dried over Na2SO4, filtered, and concentrated in vacuo. Purification by silica gel column chromatography provided the intermediate 2-methoxy-6-(2-oxoazetidin-1-yl)-1-naphthaldehyde (0.040 g, 0.16 mmol, 42%). ¹H NMR (600 MHz, CDCl₃) a 10.86 (s, 1H), 9.28 (d, J=9.4 Hz, 1H), 8.02 (d, J=9.1 Hz, 1H), 7.80 (d, J=2.2 Hz, 1H), 7.59 (dd, J=9.2, 2.1 Hz, 11H), 7.32 (d, J=9.1 Hz, 1H), 4.05 (s, 3H), 3.74 ((, J=4.5 Hz, 2H), 3.18 (t, J=4.5 Hz, 2H) ppm.

2-Hydroxy-6-(2-oxoazetidin-1-yl)-1-naphthaldehyde (BL-0738). To a solution of 2-methoxy-6-(2-oxoazetidin-1-yl)-1-naphthaldehyde (0.040 g, 0.16 mmol, 1.00 eq) in anh. CH₂Cl₂ (2.0 mL), BBr₃ (0.20 g, 0.78 mmol, 5.0) eq) was added dropwise at 0° C. under N₂. The reaction was slowly warmed to r.t. and stirred overnight. After 16 h, the reaction mixture was quenched with water and extracted with EtOAc (3×). The combined organic extracts were dried over Na₂SO₄, filtered, and concentrated in vacuo. Purification by silica gel column chromatography provided the title compound (0.011 g, 0.046 mmol. 29%). ¹H NMR (600 MHz, CDCl₃) δ 13.02 (s, 1H), 10.78 (s, 1H), 8.32 (d, J=8.9 Hz, 1H), 7.93 (d, J=8.9 Hz, 1H), 7.78 (dt, J=8.9, 1.7 Hz, 1H), 7.65 (d, J=3.0 Hz, 1H), 7.16 (d, J=8.9 Hz, 1H), 3.74 (d, J=4.6 Hz, 2H), 3.20 (s, 2H) ppm. ¹C NMR (150 MHz, CDCl₃) δ 193.31, 164.77, 164.26, 138.49, 135.42, 129.53, 128.29, 120.41, 120.02, 119.41, 114.27, 111.55, 38.39, 36.51 ppm. HRMS (ES+) calculated for [C₁₄H₁₁NO₃]+242.0813, found 242.0812.

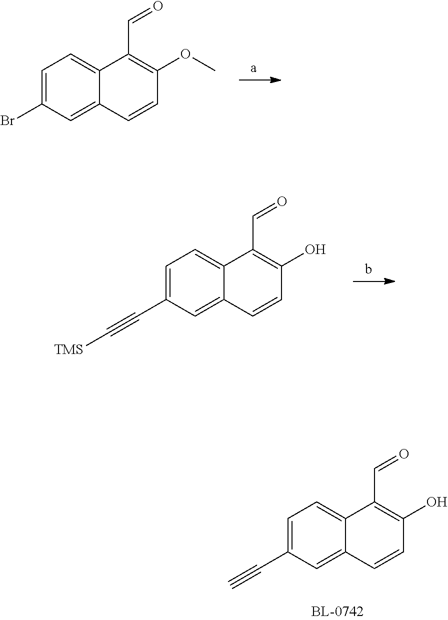 [SMILES: COc1ccc2cc(Br)ccc2c1C=O ; C[Si](C)(C)C#Cc1ccc2c(C=O)c(O)ccc2c1 ; C#Cc1ccc2c(C=O)c(O)ccc2c1]

Reagents and conditions: (a) 6-bromo-2-hydroxy-1-naphthaldehyde (1.00 eq), PdCl₂(PPh₃)₂ (0.03 eq), CuI (0.05 eq), Et₃N (23.0 eq), ethynyltrimethylsilane (1.50 eq), r.t., 16 h. (b) 2-hydroxy-6-((trimethylsilyl)ethynyl)-1-naphthaldehyde (1.00 eq), TBAF (3.50 eq), CH₃OH, r.t., 2 h.

2-Hydroxy-6-((trimethylsilyl)ethynyl)-1-naphthaldehyde. To a solution of 6-bromo-2-hydroxy-1-naphthaldehyde (0.500 g, 1.99 mmol, 1.00 eq), PdCl₂(PPh₃)₂ (0.045 g, 0.064 mmol, 0.03 eq), Cut (0.019 g, 0.100 mmol, 0.05 eq), Et₃N (4.63 g, 45.8 mmol, 23.0 eq) and ethynyltrimethylsilane (0.293 g, 2.99 mmol, 1.50 eq) were added at r.t. under N₂. The reaction mixture was stirred at r.t. overnight. The mixture was filtered through celite and rinsed with EtOAc. The volatile components were concentrated in vacuo. Purification by silica gel column chromatography provided the intermediate (0.054 g, 0.199 mmol, 10%). ¹H NMR (600 MHz, CDCl₃) δ 13.17 (d, J=2.6 Hz, 1H), 10.83-10.74 (m, 1H), 828 (dd, J=8.8, 2.9 Hz, 1H), 8.01-7.91 (m, 2H), 7.66 (dt, J=8.8, 2.2 Hz, 1H), 7.16 (dd, J=9.2, 2.6 Hz, 1H), 0.29 (s, 9H) ppm.

6-Ethynyl-2-hydroxy-1-naphthaldehyde (BL-0742). To a solution of 2-hydroxy-6-((trimethylsilyl)ethynyl)-1-naphthaldehyde (0.040 g, 0.15 mmol, 1.00 eq) in anh. CH₃OH (0.70 mL), TBAF (0.140 g, 0.52 mmol, 3.50 eq) was added at r.t. The reaction mixture was stirred at r.t. for 2 h. The reaction was quenched with water and extracted with CH₂Cl₂ (3×). The combined organic extracts were dried over Na₂SO₄, filtered, and concentrated in vacuo. Purification by reverse-phased HPLC provided the title compound (0.012 g, 0.061 mmol, 41%). ¹H NMR (600 MHz, CDCl₃) δ 13.19 (s, 1H), 10.79 (s, 1H), 8.30 (d, J=8.81 Hz, 1H), 8.00-7.92 (m, 2H), 7.68 (d, J=8.4 Hz, 1H′), 7.17 (d, J=9.2 Hz, 1H), 3.16 (s, 1H) ppm. ¹³C NMR (150 MHz, CDCl₃) δ 193.33, 165.66, 138.90, 133.60, 132.84, 132.08, 127.46, 120.27, 118.97, 118.38, 111.40, 83.15, 78.02 ppm.

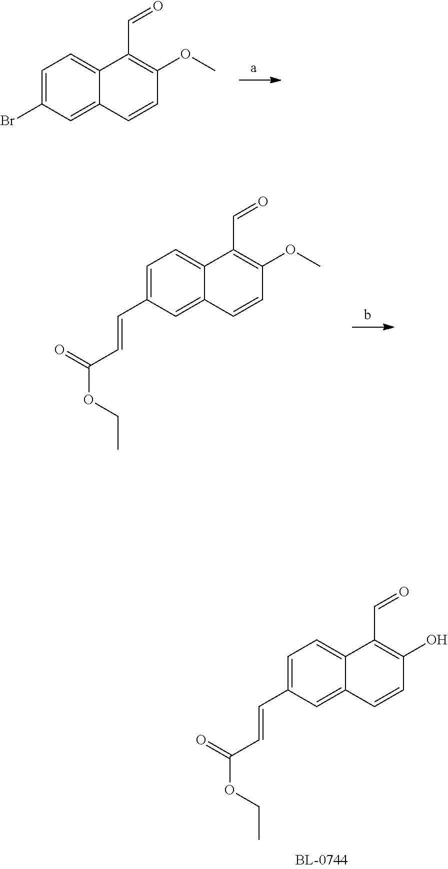 [SMILES: COc1ccc2cc(Br)ccc2c1C=O ; CCOC(=O)/C=C/c1ccc2c(C=O)c(OC)ccc2c1 ; CCOC(=O)/C=C/c1ccc2c(C=O)c(O)ccc2c1]

Reagents and conditions: (a) 6-bromo-2-methoxy-1-napthaldehyde (1.00 eq), ethyl acrylate (3.00 eq), Pd(OAc)₂ (0.03 eq), tri(o-tolyl)phosphine (0.12 eq), CH₃CN, 160° C., 30 min. (b) ethyl (E)-3-(5-formyl-6-methoxynaphthalen-2-yl)acrylate (1.00 eq), MgBr₂ (2.00 eq), NaI (2.00 eq), CH₃CN, 150° C., 2 h.

Ethyl (E)-3-(5-formyl-6-methoxynaphthalen-2-yl)acrylate. A solution of Pd(OAc)? (0.003 g, 0.011 mmol, 0.03 eq) and tri(o-tolyl)phosphine (0.014 g, 0.045 mmol, 0.12 eq) in anh. CH₃CN (0.50 mL) was stirred at r.t. for 10 min. 6-Bromo-2-methoxy-1-napthaldehyde (0.100 g, 0.377 mmol, 1.00 eq), ethyl acrylate (0.113 g, 1.13 mmol, 3.00 eq), and Et₃N (0.115 g, 1.13 mmol, 3.00 eq) were added at r.t. The reaction mixture was stirred at 160° C. for 30 min using a microwave reactor. The resulting mixture was washed with water and extracted with EtOAc (3×). The combined organic extracts were dried over Na₂SO₄, filtered, and concentrated in vacuo. Purification by silica gel column chromatography (EtOAc/Hexanes) provided the ethyl (E)-3-(5-formyl-6-methoxynaphthalen-2-yl)acrylate intermediate (0.065 g, 0.23 mmol, 61%). ¹H NMR (600 MHz, CDCl₃) δ 10.87 (s, 1H), 9.27 (d, J=9.2 Hz, 1H), 8.08 (d, J=9.2 Hz, 1H), 7.86 (d, J=1.8 Hz, 1H), 7.82-7.77 (m, 2H), 7.34 (d, J=9.2 Hz, 1H), 6.55 (d, J=16.1 Hz, 1H), 4.29 (q, J=7.0 Hz, 2H), 4.08 (s, 3H), 1.36 (t, J=7.2 Hz, 3H) ppm.

Ethyl (E)-3-(5-formyl-6-hydroxynaphthalen-2-yl)acrylate (BL-0744). To a solution of ethyl (E)-3-(5-formyl-6-methoxynaphthalen-2-yl)acrylate (0.030 g. 0.11 mmol 1.00 eq) in anh. CH₃CN (1.0 mL), magnesium bromide (0.039 g. 0.21 mmol, 2.0) eq), and sodium iodide (0.023 g, 0.21 mmol, 2.0) eq) were added at r.t. under N₂. The reaction mixture was stirred at 150° C. for 2 h. The resulting mixture was diluted with water, acidified with 1 M HCl, and extracted with EtOAc (3×). The combined organic extracts were dried over Na₂SO₄, filtered, and concentrated in vacuo. Purification by silica gel column chromatography (EtOAc/Hexanes) provided the title compound. ¹H NMR (600 MHz, CDCl₃) δ 13.19 (s, 1H), 10.81 (s, 1H), 8.36 (d, J=8.8 Hz, 1H), 8.00 (d, J=9.2 Hz, 1H), 7.89 (s, 1H), 7.84-7.76 (m, 2H), 7.18 (d, J=9.2 Hz, 1H), 6.54 (d, J=15.8 Hz, 1H), 4.29 (q, J=7.0 Hz, 2H), 1.36 (t, J=7.2 Hz, 3H) ppm. ¹³C NMR (150 MHz, CDCl₃) δ 193.36, 167.07, 165.75, 143.74, 139.54, 134.05, 130.87, 130.68, 127.87, 127.29, 120.26, 119.55, 118.64, 111.60, 60.80, 14.49 ppm. HRMS (ES+) calculated for [C₁₆H₁₄O₄]+271.0968, found 271.0965.

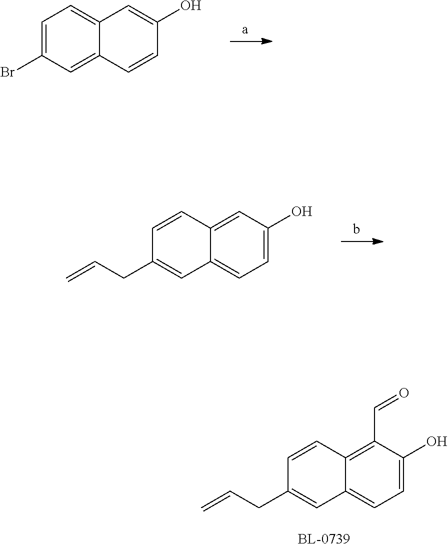 [SMILES: Oc1ccc2cc(Br)ccc2c1 ; C=CCc1ccc2cc(O)ccc2c1 ; C=CCc1ccc2c(C=O)c(O)ccc2c1]

Reagents and conditions: (a) 6-bromonaphthalen-2-ol (1.00 eq), Pd(dppf)Cl₂—CH₂Cl₂ (0.10 eq), allylmagnesium bromide (3.00 eq), THF, 0° C. to reflux, 4 h. (b) 6-allylnaphthalen-2-ol (1.00 eq), NaOH (13.0 eq). CHCl₃ (2.00 eq), 80° C., 1 h.

6-Allylnaphthalen-2-ol. General procedure B was followed allylmagnesium bromide (11.2 mL, 1.0 M solution in THF, 11.2 mmol, 5.00 eq) Purification by silica gel column chromatography (EtOAc/Hexanes) provided intermediate 6-allylnaphthalen-2-ol (0.280 g, 1.52 mmol, 68%). ¹H NMR (600 MHz, CDCl₃) δ 7.69 (dd, J=8.7, 4.7 Hz, 2H), 7.62 (d, J=8.4 Hz, 1H), 7.55 (s, 1H), 7.28 (dd, J=8.4, 2.0 Hz, 1H), 7.12 (d, J=2.6 Hz, 1H), 7.08 (dd, J=8.8, 2.6 Hz, 1H), 6.03 (ddt, J=16.7, 9.9, 6.6 Hz, 1H), 5.14-5.07 (m, 3H), 5.01 (s, 1H), 3.51 (d, J=7.3 Hz, 2H) ppm.

General Procedure C: To sodium hydroxide (13.0 eq) in water (0.3-1.0 M) was added 6-substituted naphthalen-2-ol (1.00 eq) in ethanol (0.3-1.0 M). The resulting mixture was stirred at 80° C. Chloroform (2.00 eq) was added dropwise. After stirring at 80° C. for 1 hour, the mixture was cooled to r.t. Mixture was acidified with 1 M HCl and extracted with EtOAc (3×)

6-Allyl-2-hydroxy-1-naphthaldehyde (BL-0739). General procedure C was followed using 6-allylnaphthalen-2-ol (0.150 g, 0.814 mmol, 1.00 eq). Purification by silica gel column chromatography provided the title compound (0.057 g, 0.27 mmol, 33%). ¹H NMR (600 MHz, CDCl₃) δ 13.08 (s, 1H), 10.80 (s, 1H), 8.29 (t, J=8.8 Hz, 1H), 7.94 (d, J=8.8 Hz, 1H), 7.60 (d, J=2.2 Hz, 1H), 7.48 (dd, J=8.6, 2.0 Hz, 1H), 7.13 (d, J=8.8 Hz, 1H), 6.07-5.98 (m, 1H), 5.15 (q, J=1.8 Hz, 1H), 5.12 (dq, J=3.7, 1.7 Hz, 1H), 3.54 (d, J=6.6 Hz, 2H) ppm. ¹³C NMR (150 MHz, CDCl₃) δ 193.50, 164.66, 139.02, 137.06, 136.42, 131.49, 130.66, 128.58, 128.21, 119.34, 118.88, 116.56, 111.44, 39.88 ppm. HRMS (ES+) calculated for [C₁₄H₁₂O₂]+213.0910, found 213.0912.

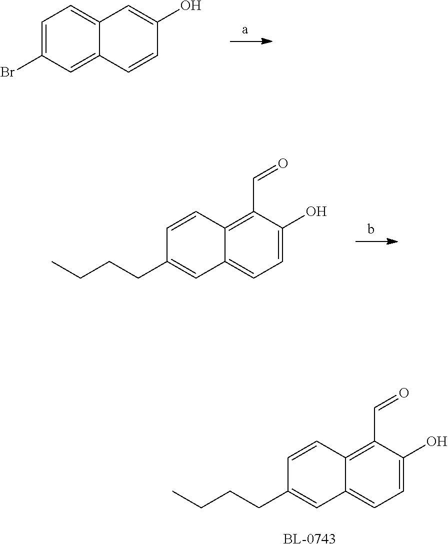 [SMILES: Oc1ccc2cc(Br)ccc2c1 ; CCCCc1ccc2c(C=O)c(O)ccc2c1 ; CCCCc1ccc2c(C=O)c(O)ccc2c1]

Reagents and conditions: (a) 6-bromonaphthalen-2-ol (1.0) eq), Pd(dppf)Cl₂—CH₂Cl₂ (0.10 eq), butylmagnesium chloride (5.00 eq), THF, 0° C. to reflux, 4 h. (b) 6-butylnaphthalen-2-ol (1.00 eq), NaOH (13.0 eq), CHCl₃ (2.00) eq), 80° C., 1 h.

6-Butylnaphthalen-2-ol. General procedure B was closely followed using butylmagnesium chloride (1.31 g, 11.2 mmol, 5.00 eq). Purification by silica gel column chromatography (EtOAc/Hexanes) provided intermediate 6-butylnaphthalen-2-ol (0.270 g, 1.35 mmol, 60%). ¹H NMR (600 MHz, CDCl₃) δ 7.68 (d, J=8.8 Hz, 1H), 7.60 (d, J=8.6 Hz, 1H), 7.53 (s, 1H), 7.28 (dd, J=8.4, 1.8 Hz, 1H), 7.12 (d, J=2.6 Hz, 1H), 7.07 (dd, J=8.7, 2.5 Hz, 1H), 5.0) (s, 11H), 2.73 (t, J=7.8 Hz, 2H), 1.66 (tt, J=9.2, 6.7 Hz, 2H), 1.38 (q, J=7.5 Hz, 2H), 0.94 (t, J=7.4 Hz, 3H ppm.

6-Butyl-2-hydroxy-1-naphthaldehyde (BL-0743). General procedure C was closely followed using 6-butylnaphthalen-2-ol (0.150 g, 0.749 mmol, 1.00 eq). Purification by silica gel column chromatography (EtOAc/Hexanes) provided the title compound (0.090 g, 0.39 mmol, 53%). ¹H NMR (600 MHz, CDCl₃) δ 13.06 (s, 1H), 10.80 (s, 1H), 8.27 (d, J=8.8 Hz, 1H), 7.93 (d, J=9.2 Hz, 1H), 7.58 (s, 1H), 7.47 (dd, J=8.6, 2.0 Hz, 1H), 7.12 (d, J=9.2 Hz, 1H), 2.76 (t, J=7.9 Hz, 2H), 1.68 (dq, J=9.2, 7.3, 6.6 Hz, 2H), 1.38 (dt, J=14.7, 7.3 Hz, 2H), 0.95 (t, J=7.3 Hz, 3H) ppm. ¹³C NMR (150 MHz, CDCl₃) δ 193.54, 164.50, 139.29, 139.02, 131.20, 130.68, 128.25, 128.20, 119.17, 118.67, 111.46, 35.37, 33.63, 22.48, 14.12 ppm. HRMS (ES+) calculated for [C₁₅H₁₆O₂]+229.1223, found 229.1223.

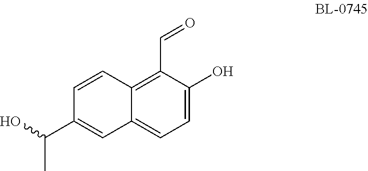 [SMILES: CC(O)c1ccc2c(C=O)c(O)ccc2c1]

2-Hydroxy-6-(I-hydroxyethyl)-1-naphthaldehyde (BL-0745). Synthesis closely followed general procedure B using 6-(1-hydroxyethyl)naphthalen-2-ol (0.150 g, 0.797 mmol, 1.00 eq). Purification by silica gel column chromatography (EtOAc/Hexanes) provided the title compound (0.101 g, 0.467 mmol, 59%). ¹H NMR (600 MHz, CDCl₃) δ 13.10 (s, 1H), 10.79 (s, 1H), 8.33 (d, J=8.4 Hz, 1H), 7.97 (d, J=9.2 Hz, 1H), 7.78 (d, J=2.2 Hz, 1H), 7.64 (dd, J=8.6, 2.0 Hz, 1H), 7.14 (d, J=8.9 Hz, 1H), 5.07 (q, J=6.6 Hz, 1H), 1.58 (d, J=6.6 Hz, 3H) ppm. ¹³C NMR (150 MHz, CDCl₃) δ 193.47, 164.94, 142.08, 139.30, 132.38, 127.87, 127.21, 125.66, 119.58, 119.15, 111.43, 70.12, 25.41 ppm.

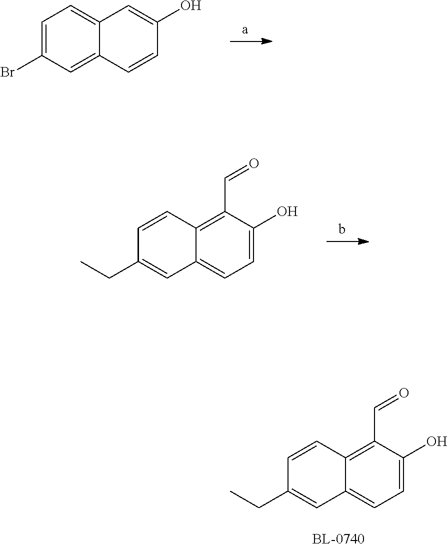

Reagents and conditions: (a) 6-bromonaphthalen-2-ol (1.00 eq), Pd(dppf)Cl₂—CH₂Cl₂ (0.10 eq), ethylmagnesium bromide (5.00 eq), THF, 0° C. to reflux, 4 h. (b) 6-ethylnaphthalen-2-ol (1.00 eq), NaOH (13.0 eq), CHCl₃ (2.00 eq), 80° C., 1 h.

6-Ethylnaphthalen-2-ol. General procedure B was closely followed using ethylmagnesium bromide (1.49 g, 11.2 mmol, 5.00 eq). Purification by silica gel column chromatography provided intermediate 6-ethylnaphthalen-2-ol (0.370 g, 2.15 mmol, 96%). ¹H NMR (600 MHz, CDCl₃) δ 7.69 (d, J=8.6 Hz, 1H), 7.61 (d, J=8.6 Hz, 1H), 7.55 (s, 1H), 7.31 (dd, J=8.5, 1.9 Hz, 1H), 7.12 (d, J=2.6 Hz, 1H), 7.07 (dd, J=8.8, 2.4 Hz, 1H), 4.98 (s, 1H), 2.77 (q, J=7.5 Hz, 2H), 1.30 (t, J=7.6 Hz, 3H) ppm.

6-Ethyl-2-hydroxy-1-naphthaldehyde (BL-0740). General procedure C was closely followed using 6-ethylnaphthalen-2-ol (0.150 g, 0.871 mmol, 1.00 eq). Purification by silica gel column chromatography (EtOAc/Hexanes) provided the title compound (0.115 g, 0.574 mmol, 66%). ¹H NMR (600 MHz, CDCl₃) δ 10.80 (s, 1H), 8.28 (d, J=8.5 Hz, 1H), 7.93 (d, J=9.1 Hz, 1H), 7.59 (d, J=1.9 Hz, 1H), 7.49 (dd, J=8.7, 2.1 Hz, 1H), 7.12 (d, J=9.1 Hz, 1H), 2.80 (q, J=7.6 Hz, 2H), 1.32 (t, J=7.6 Hz, 3H) ppm. ¹³C NMR (150 MHz, CDCl₃) δ 193.53, 164.54), 140.60, 139.02, 131.22, 130.29, 128.24, 127.54, 119.18, 118.75, 111.46, 28.62, 15.62 ppm. HRMS (ES+) calculated for [C₁₃H₁₂O₂]+201.0913, found 201.0910.

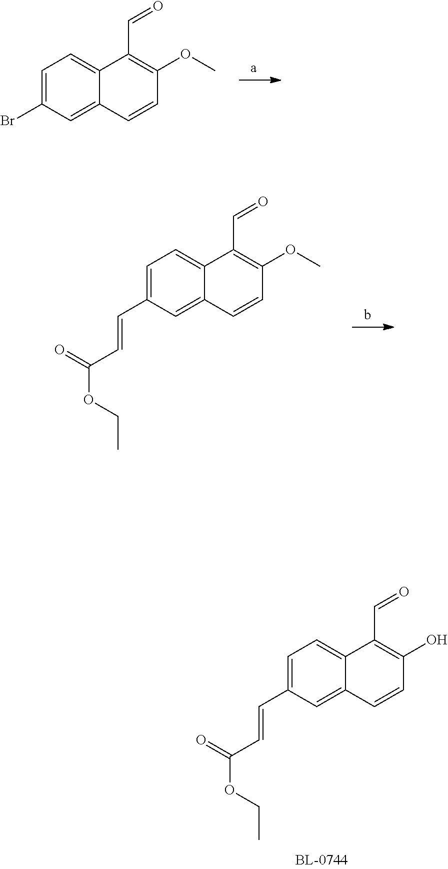 [SMILES: COc1ccc2cc(Br)ccc2c1C=O ; CCOC(=O)/C=C/c1ccc2c(C=O)c(OC)ccc2c1 ; CCOC(=O)/C=C/c1ccc2c(C=O)c(O)ccc2c1]

Reagents and conditions: (a) 6-bromo-2-methoxy-1-napthaldehyde (1.00 eq), ethyl acrylate (3.00 eq), Pd(OAc)? (0.03 eq), tri(o-tolyl)phosphine (0.12 eq), CH₃CN, 160° C., 30 min. (b) ethyl (E)-3-(5-formyl-6-methoxynaphthalen-2-yl)acrylate (1.00 eq), MgBr₂ (2.00 eq), NaI (2.00 eq), CH₃CN, 150° C., 2 h.

Ethyl (E)-3-(5-formyl-6-methoxynaphthalen-2-yl)acrylate. A solution of Pd(OAc)₂ (0.003 g, 0.011 mmol, 0.03 eq) and tri(o-tolyl)phosphine (0.014 g, 0.045 mmol, 0.12 eq) in anh. CH₃CN (0.50 mL) was stirred at r.t. for 10 min. 6-Bromo-2-methoxy-1-napthaldehyde (0.100 g, 0.377 mmol, 1.00 eq), ethyl acrylate (0.113 g, 1.13 mmol, 3.00 eq), and Et₃N (0.115 g, 1.13 mmol, 300 eq) were added at r.t. The reaction mixture was stirred at 160° C. for 30 min using a microwave reactor. The resulting mixture was washed with water and extracted with EtOAc (3×). The combined organic extracts were dried over Na2SO₄, filtered, and concentrated in vacuo. Purification by silica gel column chromatography (EtOAc/Hexanes) provided the ethyl (E)-3-(5-formyl-6-methoxynaphthalen-2-yl)acrylate intermediate (0.065 g, 0.23 mmol, 61%). ¹H NMR (600 MHz, CDCl₃) δ 10.87 (s, 11H), 9.27 (d, J=9.2 Hz, 1H), 8.08 (d, J=9.2 Hz, 1H), 7.86 (d, J=1.8 Hz, 1H), 7.82-7.77 (m, 2H), 7.34 (d, J=9.2 Hz, 1H), 6.55 (d, J=16.1 Hz, 1H), 4.29 (q, J=7.0 Hz, 21H), 4.08 (s, 3H), 1.36 (t, J=7.2 Hz, 3H) ppm.

Ethyl (E)-3-(5-formyl-6-hydroxynaphthalen-2-yl)acrylate (BL-0744). To a solution of ethyl (E)-3-(5-formyl-6-methoxynaphthalen-2-yl)acrylate (0.030 g, 0.11 mmol 1.00 eq) in anh. CH₃CN (1.0 mL), magnesium bromide (0.039 g, 0.21 mmol, 2.00 eq), and sodium iodide (0.023 g, 0.21 mmol, 2.00 eq) were added at r.t. under N₂. The reaction mixture was stirred at 150° C. for 2 h. The resulting mixture was diluted with water, acidified with 1 M HCl, and extracted with EtOAc (3×). The combined organic extracts were dried over Na2SO₄, filtered, and concentrated in vacuo. Purification by silica gel column chromatography (EtOAc/Hexanes) provided the title compound. ¹H NMR (600 MHz, CDCl3) δ 13.19 (s, 1H), 10.81 (s, 1H), 8.36 (d, J=8.8 Hz, 1H), 8.00 (d, J=9.2 Hz, 114), 7.89 (s, 1H), 7.84-7.76 (m, 2H), 7.18 (d, J=9.2 Hz, 114), 6.54 (d, J=15.8 Hz, 11H), 4.29 (q, J=7.0 Hz, 2H), 1.36 (t, J=7.2 Hz, 3H) ppm. ¹³C NMR (150 MHz, CDCl₃) δ 193.36, 167.07, 165.75, 143.74, 139.54, 134.05, 130.87, 130.68, 127.87, 127.29, 120.26, 119.55, 118.64, 111.60, 60.80, 14.49 ppm. HRMS (ES+) calculated for [C₁₆H₁₄O₄]+271.0968, found 271.0965.

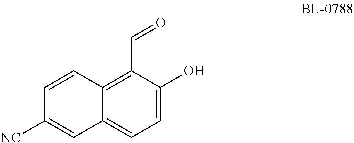 [SMILES: N#Cc1ccc2c(C=O)c(O)ccc2c1]

5-Formyl-6-hydroxy-2-naphthonitrile. General procedure C was closely followed using 6-hydroxy-2-naphthonitrile (0.100 g, 0.591 mmol, 1.00 eq). Purification by silica gel column chromatography provided the title compound (0.042 g, 0.21 mmol, 36%). ¹H NMR (600 MHz, CDCl₃) a 13.33 (d, J=3.7 Hz, 1H), 10.81 (d, J=3.7 Hz, 11H), 8.44 (dd, J=8.8, 3.7 Hz, 11H), 8.18 (d, J=4.0 Hz, 1H), 8.03 (dd, J=9.0, 3.5 Hz, 1H), 7.78 (d, J=8.8 Hz, 1H), 7.32-7.27 (m, 1H) ppm. ¹³C NMR (150 MHz, CDCl₃) δ 192.86, 166.86, 138.83, 135.06, 134.90, 130.07, 126.89, 121.52, 119.95, 118.60, 111.22, 108.25 ppm.

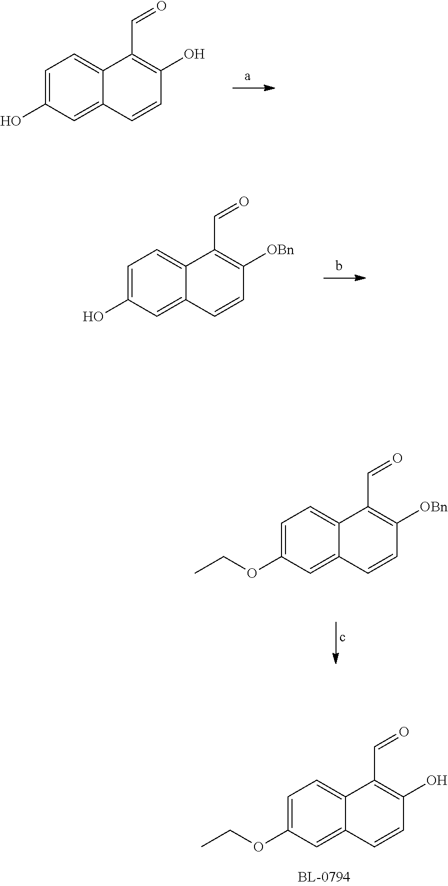 [SMILES: O=Cc1c(O)ccc2cc(O)ccc12 ; O=Cc1c(OCc2ccccc2)ccc2cc(O)ccc12 ; CCOc1ccc2c(C=O)c(OCc3ccccc3)ccc2c1 ; CCOc1ccc2c(C=O)c(O)ccc2c1]

Reagents and conditions: (a) 2,6-dihydroxy-1-naphthaldehyde (1.10 eq), K₂CO₃ (1.00 eq), benzylbromide (1.00 eq), DMF, 0° C. to r.t., 3 h. (b) of 2-(benzyloxy)-6-hydroxy-1-naphthaldehyde (1.0)), K₂CO₃ (1.20 eq), bromoethane (1.20 eq), DMF, 100° C., 16 h. (c) 2-(benzyloxy)-6-ethoxy-1-naphthaldehyde (1.00 eq), Pd/C (0.20 eq), CH₃OH, r.t., 30 min.

2-(benzyloxy)-6-hydroxy-1-naphthaldehyde. To a solution of 2,6-dihydroxy-1-naphthaldehyde (0.100 g, 0.531 mmol, 1.10 eq) in anh. DMF (5.0 mL), potassium carbonate (0.067 g, 0.483 mmol, 1.00 eq) and benzylbromide (0.083 g, 0.483 mmol, 1.00 eq) at 0° C. under N₂. The reaction mixture was slowly warmed to r.t. and stirred at this temperature until TLC indicated complete consumption of starting material. After 3 h, the reaction was diluted with water. The crude was extracted with EtOAc (3×) and washed with water (5×) and brine. The combined organic extracts were dried over anh. Na2SO4, filtered, and concentrated in vacuo. The resulting 2-(benzyloxy)-6-hydroxy-1-naphthaldehyde intermediate was used in the next step without further purification.

2-(benzyloxy)-6-ethoxy-1-naphthaldehyde. To a solution of 2-(benzyloxy)-6-hydroxy-1-naphthaldehyde (0.065 g, 0.23 mmol, 1.00 eq) in anh. DMF (2.3 mL), potassium carbonate (0.031 g, 028 mmol, 1.20 eq) and bromoethane (0.031 g. 0.28 mmol, 1.20 eq) at 0° C. under N₂. The reaction mixture was stirred at 100° C. overnight. After 16 h, the reaction was cooled to r.t. and diluted with water. The crude was extracted with EtOAc (3×) and washed with water (5×) and brine. The combined organic extracts were dried over anh. Na2SO4, filtered, and concentrated in vacuo. Purification by reversed phase HPLC (10% to 90% CH₃CN in water) provided intermediate 2-(benzyloxy)-6-ethoxy-1-naphthaldehyde (0.044 g, 0.14 mmol, 61%). ¹H NMR (600 MHz, CDCl₃) δ 10.94 (s, 1H), 9.19 (d, J=9.6 Hz, 1H), 7.91 (d, J=9.2 Hz, 1H), 7.45 (d, J=7.3 Hz, 2H), 7.41 (t, J=7.5 Hz, 2H), 7.36 (d, J=7.1 Hz, 1H), 7.33-7.29 (m, 2H), 7.07 (d, J=2.9 Hz, 1H), 5.31 (s, 2H), 4.13 (q, J=7.0 Hz, 2H), 1.47 (t, J=7.2 Hz, 3H) ppm.

6-Ethoxy-2-hydroxy-1-naphthaldehyde (BL-0794). To a solution of 2-(benzyloxy)-6-ethoxy-1-naphthaldehyde (0.044 g, 0.14 mmol, 1.0) eq) in anh. CH₃OH (1.5 mL), Pd/C (0.031 g, 10% Wt, 0.029 mmol, 0.20 eq) was added at r.t. H₂ was bubbled into the solution, and the mixture was stirred under H₂ for 30 min. The resulting mixture was filtered through celite and evaporated in vacuo. Purification by silica gel column chromatography provided the title compound (0.009 g, 0.042 mmol, 29%). ¹H NMR (600 MHz, CDCl₃) δ 12.89 (s, 1H), 10.77 (s, 11H), 8.25 (d, J=9.2 Hz, 1H), 7.87 (d, J=9.2 Hz, 1H), 7.29 (d, J=9.5 Hz, 1H), 7.15-7.10 (m, 2H), 4.14 (q, J=7.0 Hz, 2H), 1.48 (t, J=7.0 Hz, 3H) ppm. ¹³C NMR (151 MHz, CDCl₃) δ 163.30, 156.03, 138.17, 121.30, 120.24, 119.69, 109.32, 63.79, 14.94 ppm. HRMS (ES+) calculated for [C₁₃H₁₂O₃]+217.0858, found 217.0859.

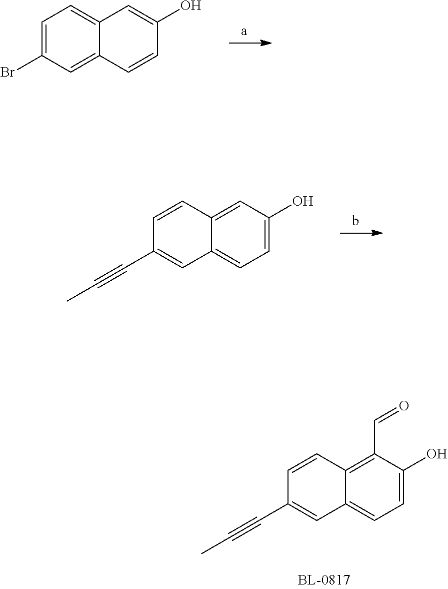 [SMILES: Oc1ccc2cc(Br)ccc2c1 ; CC#Cc1ccc2cc(O)ccc2c1 ; CC#Cc1ccc2c(C=O)c(O)ccc2c1]

Reagents and conditions: (a) 6-bromonaphthalen-2-ol (1.00 eq), Pd(dppf)Cl₂—CH₂Cl₂ (0.10 eq), propynylmagnesium bromide (3.00 eq), THF, 0° C. to reflux, 4 h. (b) 6-(prop-1-yn-1-yl)naphthalen-2-ol (1.0) eq), NaOH (13.0 eq), CHCl₃ (2.00 eq), 80° C., 1 h.

6-(Prop-1-yn-1-yl)naphthalen-2-ol. General procedure B was followed using propynylmagnesium bromide (0.482 g, 3.36 mmol, 3.00 eq). Purification by silica gel column chromatography (EtOAc/Hexanes) provided the 6-(prop-1-yn-1-yl)naphthalen-2-ol intermediate (0.186 g, 1.02 mmol, 91%), ¹H NMR (600 MHz, CDCl₃) δ 7.82 (s 1H), 7.68 (d, J=9.0 Hz, 1H), 7.58 (d, J=8.3 Hz, 1H), 7.41-7.38 (m, 1H), 7.09 (dd, J=11.9, 3.2 Hz, 21H), 4.92 (s, 1H), 2.09 (s, 3H) ppm.

2-Hydroxy-6-(prop-1-yn-1-yl)-1-naphthaldehyde (BL-0817). General procedure C was followed using 6-(prop-1-yn-1-yl)naphthalen-2-ol (0.100 g, 0.549 mmol, 1.00 eq). Purification by silica gel column chromatography provided the title compound (0.039 g, 0.19 mmol, 34%). ¹H NMR (600 MHz, CDCl₃) δ 13.12 (s, 1H), 10.77 (s, 1H), 8.25 (d, J=8.8 Hz, 1H), 7.90 (d, J=9.1 Hz, 1H), 7.84 (d, J=2.2 Hz, 1H), 7.59 (dd, J=8.8, 1.8 Hz, 1H), 7.14 (d, J=8.8 Hz, 1H), 2.10 (s, 3H) ppm. ¹³C NMR (150 MHz, CDCl₃) δ 193.36, 165.26, 138.82, 132.44, 132.09, 127.70, 120.43, 119.94, 118.79, 111.46, 86.80, 79.26, 19.86 ppm. HRMS (ES−) calculated for [C₁₄H₁₀O₂]⁻ 209.0608, found 209.0609.

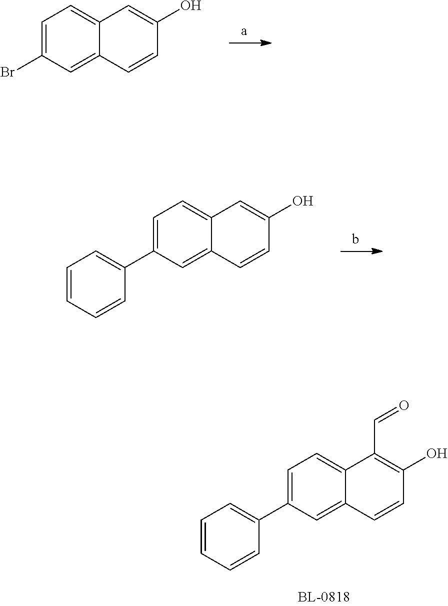 [SMILES: Oc1ccc2cc(Br)ccc2c1 ; Oc1ccc2cc(-c3ccccc3)ccc2c1 ; O=Cc1c(O)ccc2cc(-c3ccccc3)ccc12]

Reagents and conditions: (a) 6-bromo-2-naphthol (1.00 eq), phenylboronic acid (1.00 eq), Pd(OAc)₂ (0.10 eq), K₂CO₃ (3.00 eq), DMF, 30° C., 16 h. (b) 6-phenylnaphthalen-2-ol (1.00 eq), NaOH (13.0 eq), CHCl₃ (2.00 eq), 80° C., 1 h.

6-Phenylnaphthalen-2-ol. To a solution of 6-bromo-2-naphthol (0.200 g, 0.897 mmol, 1.00 eq) and phenylboronic acid (0.109 g, 0.897 mmol, 1.00 eq) in anh. DMF (5 mL), Pd(OAc)₂ (0.021 g, 0.090 mmol, 0.10 eq) and potassium carbonate (0.372 g, 2.69 mmol, 3.00 eq) in water (4.0 mL) were added at r.t. under N₂. The resulting mixture was stirred at 30° C. for 16 h. The resulting mixture was cooled to r.t., filtered through celite, and diluted with NH₄Cl. The crude was extracted with EtOAc (3×), washed with water (5×) and brine. The combined organic extracts were dried over anh. Na₂SO₄, filtered, and concentrated in vacuo. Purification by silica gel column chromatography provided the 6-phenylnaphthalen-2-ol intermediate (0.102 g, 0.463 mmol, 52%). ¹H NMR (600 MHz, CDCl₃) δ 7.97 (d, J=2.2 Hz, 1H), 7.81 (d, J=8.8 Hz, 1H), 7.76 (d, J=8.6 Hz, 1H), 7.73-7.68 (m, 3H), 7.50-7.45 (m, 2H), 7.38-7.34 (m, 1H), 7.18 (d, J=2.6 Hz, 11H), 7.13 (dd, J=8.8, 2.4 Hz, 1H) ppm.

2-Hydroxy-6-phenyl-1-naphthaldehyde (BL-0818). General procedure C was followed using 6-phenylnaphthalen-2-ol (0.050 g, 0.23 mmol, 1.0) eq). Purification by silica gel column chromatography provided the title compound (0.023 g, 0.093 mmol, 41%). ¹H NMR (600 MHz, CDCl₃) & 13.15 (s, 1H), 10.86 (s, 1H), 8.43 (d, J=8.8 Hz, 1H), 8.05 (d, J=8.8 Hz, 1H), 8.01 (d, J=2.2 Hz, 1H), 7.89 (dd, J=8.8, 2.2 Hz, 1H), 7.73-7.68 (m, 2H), 7.50 (t, J=7.8 Hz, 2H), 7.40 (t, J=7.5 Hz, 1H), 7.19 (d, J=9.1 Hz, 1H) ppm. ¹³C NMR (150 MHz, CDCl₃) S 193.41, 165.07, 140.31, 139.47, 137.55, 132.18, 129.15, 128.73, 128.37, 127.75, 127.38, 127.28, 127.27, 119.81, 119.38, 111.47 ppm. HRMS (ES−) calculated for [C₁₇H₁₂O₂]⁻ 247.0565, found 247.0563.

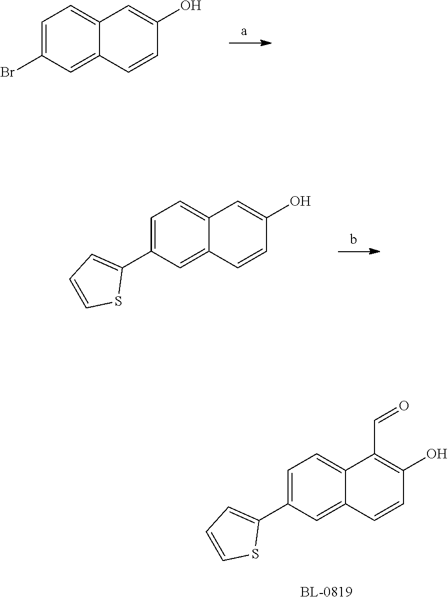 [SMILES: Oc1ccc2cc(Br)ccc2c1 ; Oc1ccc2cc(-c3cccs3)ccc2c1 ; O=Cc1c(O)ccc2cc(-c3cccs3)ccc12]

Reagents and conditions: (a) 6-bromo-2-naphthol (1.00 eq), thiophenyl-2-boronic acid (2.00 eq), Pd(OAc)₂ (0.10 eq), K₂CO₃ (3.00 eq), DME:Water:EtOH (7/312), 30° C., 16 h. (b6-(thiophen-2-yl)naphthalen-2-ol (1.00 eq), NaOH (13.0 eq), CHCl₃ (2.00 eq), 80° C., 1 h.

6-(Thiophen-2-yl)naphthalen-2-ol. A solution of 6-bromo-2-naphthol (0.100 g, 0.448 mmol, 1.00 eq), thiophenyl-2-boronic acid (0.115 g, 0.897 mmol, 2.0) eq), potassium carbonate (0.186 g, 1.34 mmol, 3.00 eq), Pd(OAc)₂ (0.011 g, 0.045 mmol, 0.10 eq) in a 7/3/2 mixture of DME:Water:Ethanol (˜4 mL) was heated at 150° C., 100 W using a microwave reactor for 5 min. The resulting mixture was filtered through celite, washed with NH₄Cl, and extracted with EtOAc (3×). The combined organic extracts were dried over anh. Na₂SO4, filtered, and concentrated in vacuo. The resulting brown crude was purified by silica gel column chromatography (up to 20% EtOAc in Hexanes) to yield the desired product as an off white solid (0.055 g, 0.045 mol, 55%). ¹H NMR (600 MHz, CDCl₃) δ 7.98 (s, 1H), 7.77 (d, J=8.6 Hz, 1H), 7.74-7.67 (m, 2H), 7.40-7.38 (m, 1H), 7.30 (t, J=3.4 Hz, 1H), 7.14 (d, J=2.9 Hz, 1H), 7.13-7.10 (m, 2H), 4.92 (s, 1H) ppm.

2-Hydroxy-6-(thiophen-2-yl)-1-naphthaldehyde (BL-0819). General procedure C was followed using 6-(thiophen-2-yl)naphthalen-2-ol (0.045 g, 0.20 mmol, 1.0) eq). Purification by silica gel column chromatography provided the title compound (0.014 g, 0.055 mmol, 28%). ¹H NMR (60) MHz, CDCl₃) δ 13.11 (s, 1H), 10.80 (s, 1H), 8.34 (d, J=8.8 Hz, 1H), 7.99 (d, J=9.1 Hz, 2H), 7.87 (dd, J=8.8, 2.2 Hz, 1H), 7.41 (d, J=3.7 Hz, 1H), 7.34 (d, J=5.1 Hz, 1H), 7.16 (d, J=9.0 Hz, 1H), 7.13 (dd, J=4.7, 3.7 Hz, 1H) ppm. ¹³C NMR (150 MHz, CDCl₃) S 164.88, 143.49, 139.04, 132.12, 130.85, 128.48, 128.17, 127.47, 125.73, 120.14, 119.74, 119.33, 111.48 ppm. HRMS (ES−) calculated for [C₁₅H₁₀O₂S]⁻ 253.0329, found 253.0327.

### REFERENCES

1. Kleaveland, B., Zheng, X., Liu, J. J., Blum, Y., Tung, J. J., Zou, Z., Sweeney, S. M., Chen, M., Guo, L., Lu. M. M., Zhou, D., Kitajewski, J., Affolter, M., Ginsberg, M. H., and Kahn, M. L. (2009) Regulation of cardiovascular development and integrity by the heart of glass-cerebral cavernous malformation protein pathway. Nat Med 15, 169-176
2. Gingras, A. R., Liu, J. J., and Ginsberg, M. H. (2012) Structural basis of the junctional anchorage of the cerebral cavernous malformations complex. J Cell Biol 199, 39-48
3. Gingras, A. R., Puzon-McLaughlin, W., and Ginsberg, M. H. (2013) The structure of the ternary complex of Krev interaction trapped 1 (KRIT1) bound to both the Rap1 GTPase and the heart of glass (HEG1) cytoplasmic tail. J Biol Chem 288, 23639-23649
4. Glading, A., Han, J., Stockton, R. A., and Ginsberg, M. H. (2007) KRIT-1/CCM1 is a Rap1 effector that regulates endothelial cell cell junctions. J Cell Biol 179, 247-254
5. Gingras, A. R., Liu, J. J., and Ginsberg, M. H. (2012) Structural basis of the junctional anchorage of the cerebral cavernous malformations complex. The Journal of cell biology 199, 39-48
6. Li, X., Zhang, R., Draheim, K. M., Liu, W., Calderwood, D. A., and Boggon, T. J. (2012) Structural Basis for Small G Protein Effector Interaction of Ras-related Protein 1 (Rapt) and Adaptor Protein Krev Interaction Trapped 1 (KRIT1). The Journal of biological chemistry 287, 22317-22327
7. Donat, S., Lourenco, M., Paolini, A., Otten, C., Renz, M., and Abdelilah-Seyfried, S. (2018) Heg1 and Ccm1/2 proteins control endocardial mechanosensitivity during zebrafish valvulogenesis. Elife 7
8. Mably, J. D., Mohideen, M. A., Burns. C. G., Chen, J. N., and Fishman, M. C. (2003) heart of glass regulates the concentric growth of the heart in zebrafish. Current biology: CB 13, 2138-2147
9. Whitehead, K. J., Plummer, N. W., Adams, J. A., Marchuk, D. A., and Li, D. Y. (2004) Ccm1 is required for arterial morphogenesis: implications for the etiology of human cavernous malformations. Development 131, 1437-1448
10. Hogan, B. M., Bussmann, J., Wolburg, H., and Schulte-Merker, S. (2008) ccm1 cell autonomously regulates endothelial cellular morphogenesis and vascular tubulogenesis in zebrafish. Hum Mol Genet 17, 2424-2432
11. Mably, J. D., Chuang, L. P., Serluca, F. C., Mohideen, M. A., Chen, J. N., and Fishman, M. C. (2006) santa and valentine pattern concentric growth of cardiac myocardium in the zebrafish. Development 133, 3139-3146
12. Cuttano, R., Rudini, N., Bravi, L., Corada, M., Giampietro, C., Papa, E., Morini, M. F., Maddaluno, L., Baeyens, N., Adams, R. H., Jain, M. K., Owens, G. K., Schwartz, M., Lampugnani, M. G., and Dejana, E. (2016) KLF4 is a key determinant in the development and progression of cerebral cavernous malformations. EMBO molecular medicine 8, 6-24
13. Bharadwaj, A. S., Kelly, M., Kim, D., and Chao, H. (2010) Induction of immune tolerance to FIX by intramuscular AAV gene transfer is independent of the activation status of dendritic cells. Blood 115, 500-509
14. Zhou, Z., Tang, A. T., Wong, W. Y., Bamezai, S., Goddard, L. M., Shenkar, R., Zhou, S., Yang, J., Wright, A. C., Foley, M., Arthur, J. S., Whitehead, K. J., Awad, I. A., Li, D. Y., Zheng, X., and Kahn, M. L. (2016) Corrigendum: Cerebral cavernous malformations arise from endothelial gain of MEKK3-KLF2/4 signalling. Nature 536, 488
15. Lopez-Ramirez, M. A., Fonseca, G., Zeineddine, H. A., Girard, R., Moore, T., Pham, A., Cao. Y., Shenkar, R., de Kreuk, B. J., Lagarrigue, F., Lawler, J., Glass, C. K., Awad, I. A., and Ginsberg, M. H. (2017) Thrombospondin1 (TSP1) replacement prevents cerebral cavernous malformations. J Exp Med 214, 3331-3346
16. Nowak, D., Ogawa, S., Muschen, M., Kato, M., Kawamata, N., Meixel, A., Nowak, V., Kim, H. S., Kang, S., Paquette, R., Chang, M. S., Thoennissen, N. H., Mossner. M., Hofmann, W. K., Kohlmann, A., Weiss, T., Haferlach, T., Haferlach. C., and Koeftier, H. P. (2010) SNP array analysis of tyrosine kinase inhibitor-resistant chronic myeloid leukemia identifies heterogeneous secondary genomic alterations. Blood 115, 1049-1053
17. Fisher, O. S., Deng, H., Liu, D., Zhang, Y., Wei, R., Deng, Y., Zhang, F., Louvi, A., Turk, B. E., Boggon, T. J., and Su, B. (2015) Structure and vascular function of MEKK3-cerebral cavernous malformations 2 complex. Nat Commun 6, 7937
18. Chistiakov, D. A., Orekhov, A. N., and Bobryshev, Y. V. (2017) Effects of shear stress on endothelial cells: go with the flow. Acta physiologica 219, 382-408
19. Gore, A. V., Monzo, K., Cha, Y. R., Pan, W., and Weinstein, B. M. (2012) Vascular development in the zebrafish. Cold Spring Harbor perspectives in medicine 2, a006684
20. Huddleson, J. P., Ahmad, N., Srinivasan, S., and Lingrel, J. B. (2005) Induction of KLF2 by fluid shear stress requires a novel promoter element activated by a phosphatidylinositol 3-kinase-dependent chromatin-remodeling pathway. J Biol Chem 280, 23371-23379
21. Parmar, K. M., Nambudiri, V., Dai, G., Larman, H. B., Gimbrone, M. A., Jr., and Garcia-Cardena, G. (2005) Statins exert endothelial atheroprotective effects via the KLF2 transcription factor. J Biol Chem 280, 26714-26719
22. Coon, B. G., Baeyens, N., Han, J., Budatha, M., Ross, T. D., Fang, J. S., Yun, S., Thomas, J. L., and Schwartz. M. A. (2015) Intramembrane binding of VE-cadherin to VEGFR2 and VEGFR3 assembles the endothelial mechanosensory complex. J Cell Biol 208, 975-986
23. DiStefano, P. V., and Glading, A. J. (2020) VEGF signalling enhances lesion burden in KRIT1 deficient mice. Journal of cellular and molecular medicine 24, 632-639
24. Gingras, A. R., Puzon-McLaughlin, W., Bobkov, A. A., and Ginsberg, M. H. (2016) Structural Basis of Dimeric Rasip 1 RA Domain Recognition of the Ras Subfamily of GTP-Binding Proteins. Structure 24, 2152-2162
25. Tucker, J., Sczakiel, G., Feuerstein, J., John, J., Goody, R. S., and Wittinghofer, A. (1986) Expression of p21 proteins in Escherichia coli and stereochemistry of the nucleotide-binding site. EMBO J 5, 1351-1358
26. de Kreuk, B. J., Gingras, A. R., Knight, J. D., Liu, J. J., Gingras, A. C., and Ginsberg, M. H. (2016) Heart of glass anchors Rasip1 at endothelial cell-cell junctions to support vascular integrity. Elife 5
27. Edwards, B. S., and Sklar, L. A. (2015) Flow Cytometry: Impact on Early Drug Discovery. Journal of biomolecular screening 20, 689-707
28. Zhang, J. H., Chung, T. D., and Oldenburg, K. R. (1999) A Simple Statistical Parameter for Use in Evaluation and Validation of High Throughput Screening Assays. Journal of biomolecular screening 4, 67-73
29. Kabsch, W. (2010) Xds. Acta Crystallogr D Biol Crystallogr 66, 125-132
30. (1994) The CCP4 suite: programs for protein crystallography. Acta Crystallogr D Biol Crystallogr 50, 760-763
31. Lopez-Ramirez, M. A., Fischer, R., Torres-Badillo, C. C., Davies, H. A., Logan, K., Pfizenmaier, K., Male, D. K., Sharrack, B., and Romero, I. A. (2012) Role of caspases in cytokine-induced barrier breakdown in human brain endothelial cells. J Immunol 189, 3130-3139
32. Heckel, E., Boselli, F., Roth, S., Krudewig, A., Belting, H. G., Charvin, G., and Vermot, J. (2015) Oscillatory Flow Modulates Mechanosensitive klf2a Expression through trpv4 and trpp2 during Heart Valve Development. Curr Biol 25, 1354-1361
33. Kanda, T., Sullivan, K. F., and Wahl, G. M. (1998) Histone-GFP fusion protein enables sensitive analysis of chromosome dynamics in living mammalian cells. Curr Biol 8, 377-385
34. Hopkins. A. L., Keseru, G. M., Leeson, P. D., Rees, D. C., and Reynolds, C. H. (2014) The role of ligand efficiency metrics in drug discovery. Nature reviews. Drug discovery 13, 105-121
35. Johnson, T. W., Gallego, R. A., and Edwards, M. P. (2018) Lipophilic Efficiency as an Important Metric in Drug Design. J Med Chem 61, 6401-6420
36. Sako, K., Fukuhara, S., Minami, T., Hamakubo, T., Song, H., Kodama, T., Fukamizu, A., Gutkind, J. S., Koh, G. Y., and Mochizuki, N. (2009) Angiopoietin-1 induces Kruppel-like factor 2 expression through a phosphoinositide 3-kinase/AKT-dependent activation of myocyte enhancer factor 2. J Biol Chem 284, 5592-5601
37. Nayak, L., Lin, Z., and Jain, M. K. (2011) “Go with the flow”: how Kruppel-like factor 2 regulates the vasoprotective effects of shear stress. Antioxid Redox Signal 15, 1449-1461
38. Gore, A. V., Lampugnani, M, G., Dye, L., Dejana, E., and Weinstein, B. M. (2008) Combinatorial interaction between CCM pathway genes precipitates hemorrhagic stroke. Dis Model Mech 1, 275-281
39. Kozakov, D., Hall, D. R., Jehle, S., Luo, L., Ochiana, S. O., Jones, E. V., Pollastri, M., Allen, K. N., Whitty, A., and Vajda, S. (2015) Ligand deconstruction: Why some fragment binding positions are conserved and others are not. Proc Natl Acad Sci USA 112, E2585-2594
40. Park, J., Fu, H., and Pei, D. (2003) Peptidyl aldehydes as reversible covalent inhibitors of SRC homology 2 domains. Biochemistry 42, 5159-5167
41. Hajduk, P. J., and Greer, J. (2007) A decade of fragment-based drug design: strategic advances and lessons learned. Nature reviews. Drug discovery 6, 211-219
42. Jin, Z. G., Ueba, H., Tanimoto, T., Lungu, A. O., Frame, M. D., and Berk, B. C. (2003) Ligand-independent activation of vascular endothelial growth factor receptor 2 by fluid shear stress regulates activation of endothelial nitric oxide synthase. Circ Res 93, 354-363
43. Jin, Z. G., Wong, C., Wu, J., and Berk, B. C. (2005) Flow shear stress stimulates Gab1 tyrosine phosphorylation to mediate protein kinase B and endothelial nitric-oxide synthase activation in endothelial cells. J Biol Chem 280, 12305-12309
44. Baeyens, N., Bandyopadhyay, C., Coon, B. G., Yun, S., and Schwartz, M. A. (2016) Endothelial fluid shear stress sensing in vascular health and disease. J Clin Invest 126, 821-828
45. Lakshmikanthan, S., Zheng, X., Nishijima, Y., Sobezak, M., Szabo, A., Vasquez-Vivar, J., Zhang, D. X., and Chrzanowska-Wodnicka, M. (2015) Rap1 promotes endothelial mechanosensing complex formation, NO release and normal endothelial function. EMBO Rep 16, 628-637
46. Cullere, X., Plovie, E., Bennett, P. M., MacRae, C. A., and Mayadas, T. N. (2015) The cerebral cavernous malformation proteins CCM2L and CCM2 prevent the activation of the MAP kinase MEKK3. Proc Natl Acad Sci USA 112, 14284-14289
47. Maddaluno L, et al. (2013) EndMT contributes to the onset and progression of cerebral cavernous malformations. Nature 498(7455):492-496.
48. Renz M, et al. (2015) Regulation of beta1 integrin-Klf2-mediated angiogenesis by CCM proteins. Dev Cell 32(2):181-190.
49. Atkins G B & Jain M K (2007) Role of Kruppel-like transcription factors in endothelial biology. Circ Res 100(12):1686-1695.
50. Alaiti M A, Orasanu G, Tugal D, Lu Y, & Jain M K (2012) Kruppel-like factors and vascular inflammation, implications for atherosclerosis. Current atherosclerosis reports 14(5):438449.

The claims are provided as follows:

1. A method of treating a disease in a subject by reducing thrombosis, atherosclerosis, or inflammation comprising administering to a subject in need an effective amount of a Siritol compound or salt thereof that binds to KRIT1 FERM domain to inhibit binding with HEG1.

2. The method of claim 1, wherein the disease is rheumatoid arthritis, gout, spondyloarthritis, vasculitis, adult respiratory distress syndrome, post-perfusion injury, glomerulonephritis, cytokine storm, myocardial infarction, stroke, deep vein thrombosis, pulmonary embolus, thrombotic thrombocytopenic purpura, COVID-19, coronary artery disease, carotid atherosclerosis, cerebrovascular disease, vascular dementia, or aortic aneurysm.

3. The method of claim 1, wherein the compound is a compound or Formula (A) or Formula (B), a salt thereof, or a salt hydrate thereof; 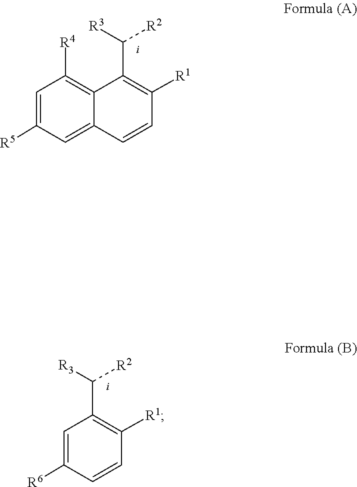
wherein R¹ is selected from the group consisting of hydroxyl and hydrogen;
wherein R² is selected from the group consisting of oxygen and nitrogen, wherein the nitrogen is substituted with (a) R^{a} or (b) R^{a} and R^{b}, wherein i is (i) a single bond, a double bond, or a triple bond when R² is nitrogen, or (ii) a double bond when R² is oxygen;
wherein R³ is selected from the group consisting of hydrogen and a C₁-C₂₀ hydrocarbyl;
wherein R⁴ is selected from the group consisting of hydrogen, hydroxyl, nitrogen, and oxygen, wherein the oxygen is substituted with R^{c}, and the nitrogen is substituted with (i) R^{d} or (ii) R^{d} and R^{e};
wherein R⁵ is selected from the group consisting of (i) hydrogen, (ii) hydroxyl, (iii) a C₁-C₂₀ hydrocarbyl, (iv) a halogen, (v) nitrogen, and (vi) oxygen, wherein the oxygen substituted with R^{f}, and the nitrogen is substituted with (a) R^{g} or (b) R^{g} and R^{h}; and
wherein R⁶ is selected from the group consisting of hydrogen and a C₁-C₂₀ hydrocarbyl;
wherein R^{c}, and R^{f} are independently selected from a C₁-C₂₀ hydrocarbyl, and
wherein R^{a}, R^{b}, R^{d}, R^{e}, R^{g} and R^{h} are independently selected from hydrogen and a C₁-C₂₀ hydrocarbyl.

4. The method of claim 3, wherein the compound is selected from the group consisting of HKi1, HKi2, HKi5, BL-0549, BL-0558, BL-0552, BL-0628, BL-0661, BL-0666, BL-0670, BL-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

5. The method of claim 3, wherein the Sirtinol derivative comprises an aldehyde moiety.

6. The method of claim 1, wherein the administering upregulates endothelial nitric oxide synthase, thrombomodulin 1, vascular endothelial growth factor A, Thrombospondin 1, Monocyte chemoattractant protein, or C-X-C chemokine receptor type 4.

7. The method of claim 1, wherein the administering upregulates PI3K/Akt signaling.

8. The method of claim 1, wherein the compound occupies a HEG1 binding pocket of KRIT1.

9. The method of claim 1, wherein the administering induces expression of KLF2 or KLF4.

10. A method of improving laminar blood-flow in a subject comprising administering to a subject in need an effective amount of a Sirtinol compound or salt thereof that binds to KRIT1 FERM domain to inhibit binding with HEG1.

11. The method of claim 10, wherein the compound is selected from the group consisting of HKi1, HKi2, HKi5, BL-0549, BL-0558, BL-0552, BL-0628, BL-0661, BL-0666, BL-0670, BL-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

12. A compound or Formula (A) or Formula (B), a salt thereof, or a salt hydrate thereof; 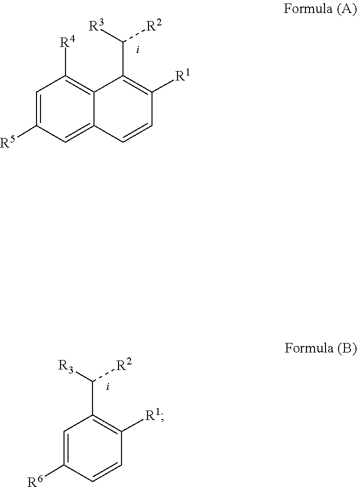
wherein R¹ is selected from the group consisting of hydroxyl and hydrogen;
wherein R² is selected from the group consisting of oxygen and nitrogen, wherein the nitrogen is substituted with (a) R^{a} or (b) R^{a} and R^{b}, wherein i is (i) a single bond, a double bond, or a triple bond when R² is nitrogen, or (ii) a double bond when R² is oxygen;
wherein R³ is selected from the group consisting of hydrogen and a C₁-C₂₀ hydrocarbyl;
wherein R⁴ is selected from the group consisting of hydrogen, hydroxyl, nitrogen, and oxygen, wherein the oxygen is substituted with R^{c}, and the nitrogen is substituted with (i) R^{d} or (ii) R^{d} and R^{e};
wherein R⁵ is selected from the group consisting of (i) hydrogen, (ii) hydroxyl, (iii) a C₁-C₂₀ hydrocarbyl, (iv) a halogen, (v) nitrogen, and (vi) oxygen, wherein the oxygen substituted with R^{f}, and the nitrogen is substituted with (a) R^{g} or (b) R^{g} and R^{h}; and
wherein R⁶ is selected from the group consisting of hydrogen and a C₁-C₂₀ hydrocarbyl;
wherein R^{c}, and R^{f} are independently selected from a C₁-C₂₀ hydrocarbyl, and
wherein R^{a}, R^{b}, R^{d}, R^{e}, R^{g} and R^{h} are independently selected from hydrogen and a C₁-C₂₀ hydrocarbyl.

13. The compound or Formula (A) or Formula (B), a salt thereof, or a salt hydrate thereof of claim 12, wherein R^{c}, and R^{f} are independently selected from a C₁-C₁₀ hydrocarbyl, and
wherein R^{a}, R^{b}, R^{d}, R^{e}, R^{g} and R^{h} are independently selected from hydrogen and a C₁-C₁₀ hydrocarbyl.

14. The compound or Formula (A) or Formula (B), a salt thereof, or a salt hydrate thereof of claim 12, wherein R^{c}, and R^{f} are independently selected from a C₁-C₆ hydrocarbyl, and wherein R^{a}, R^{b}, R^{d}, R^{e}, R^{g} and R^{h} are independently selected from hydrogen and a C₁-C₆ hydrocarbyl.

15. The compound or Formula (A) or Formula (B), a salt thereof, or a salt hydrate thereof of claim 12, wherein R¹ is hydroxyl, and R⁴ and R⁵ are hydrogen.

16. The compound of claim 15, wherein R² is nitrogen, R³ is hydrogen, and i is a double bond.

17. The compound of claim 16, wherein R^{a} is selected from the group consisting of o-benzoic acid, m-benzoic acid, p-benzoic acid, and 5-(1H-tetrazole).

18. The compound of claim 15, wherein R² is oxygen, R³ is hydrogen, and i is a double bond.

19. The compound of claim 18, wherein R⁵ is hydroxyl.

20. The compound of claim 19, wherein R⁵ is a methyl.

21. The compound of claim 19, wherein R⁵ is oxygen.

22. A pharmaceutical composition comprising a treatment effective amount of a compound chosen from the group consisting of Formula (A) or Formula (B), a salt thereof, or a salt hydrate thereof of claim 12.

23. The pharmaceutical composition of claim 22, wherein the compound is chosen from the group consisting of HKi3, BL-0549, BL-0558, BL-0552, BL-0628, BL-0661, BL-0666, BL-0670, BL-0691, BL-0693, BL-0700, BL-702, BL-0736, BL-0737, BL-0738, BL-0739, BL-0740, BL-0742, BL-0743, BL-0744, BL-0745, BL-0788, BL-0794, BL-0817, BL-0818, and BL-0819.

24. The pharmaceutical composition of claim 22, wherein the composition is used to reduce thrombosis, atherosclerosis, or inflammation in a subject in need.

25. The compound or Formula (A) or Formula (B), a salt thereof, or a salt hydrate thereof of claim 12, wherein the HEG1-KRIT1 protein complex is inhibited.
